SEQUENCE LISTING (122 sequences)

SEQ ID NO: 1 - RNA, 41 nt, synthetic construct
  source 1..41: mol_type = other RNA
gggttgagcgtctgatgaggccagtaggccgaaaggaagtt

SEQ ID NO: 2 - RNA, 41 nt, synthetic construct
  source 1..41: mol_type = other RNA
gggtagagcgtctgatgaggccagtaggccgaaaggaagtt

SEQ ID NO: 3 - RNA, 41 nt, synthetic construct
  source 1..41: mol_type = other RNA
gggttgagcgtctgatgaggccnnnnggccgaaaggaagtt

SEQ ID NO: 4 - RNA, 41 nt, synthetic construct; identical to SEQ ID NO: 1
  source 1..41: mol_type = other RNA

SEQ ID NO: 5 - RNA, 41 nt, synthetic construct
  source 1..41: mol_type = other RNA
gggttgagcgtctgatgaggcctaaaggccgaaaggaagtt

SEQ ID NO: 6 - RNA, 41 nt, synthetic construct
  source 1..41: mol_type = other RNA
gggttgagcgtctgatgaggccaaaaggccgaaaggaagtt

SEQ ID NO: 7 - RNA, 41 nt, synthetic construct
  source 1..41: mol_type = other RNA
gggttgagcgtctgatgaggccgtgaggccgaaaggaagtt

SEQ ID NO: 8 - RNA, 41 nt, synthetic construct
  source 1..41: mol_type = other RNA
gggttgagcgtctgatgaggccgagaggccgaaaggaagtt

SEQ ID NO: 9 - RNA, 41 nt, synthetic construct
  source 1..41: mol_type = other RNA
gggttgagcgtctgatgaggccgttaggccgaaaggaagtt

SEQ ID NO: 10 - RNA, 41 nt, synthetic construct
  source 1..41: mol_type = other RNA
gggttgagcgtctgatgaggccgataggccgaaaggaagtt

SEQ ID NO: 11 - RNA, 41 nt, synthetic construct
  source 1..41: mol_type = other RNA
gggttgagcgtctgatgaggccgacaggccgaaaggaagtt

SEQ ID NO: 12 - RNA, 41 nt, synthetic construct
  source 1..41: mol_type = other RNA
gggttgagcgtctgatgaggccaaagggccgaaaggaagtt

SEQ ID NO: 13 - RNA, 41 nt, synthetic construct
  source 1..41: mol_type = other RNA
gggttgagcgtctgatgaggccaacaggccgaaaggaagtt

SEQ ID NO: 14 - RNA, 41 nt, synthetic construct
  source 1..41: mol_type = other RNA
gggttgagcgtctgatgaggccattaggccgaaaggaagtt

SEQ ID NO: 15 - RNA, 41 nt, synthetic construct
  source 1..41: mol_type = other RNA
gggttgagcgtctgatgaggccaataggccgaaaggaagtt

SEQ ID NO: 16 - RNA, 41 nt, synthetic construct
  source 1..41: mol_type = other RNA
gggttgagcgtctgatgaggccagcaggccgaaaggaagtt

SEQ ID NO: 17 - RNA, 15 nt, synthetic construct
  source 1..15: mol_type = other RNA
acttcctcacgctct

SEQ ID NO: 18 - RNA, 12 nt, synthetic construct
  source 1..12: mol_type = other RNA
gtccagtaggac

SEQ ID NO: 19 - RNA, 14 nt, synthetic construct
  source 1..14: mol_type = other RNA
ggcccagtagggcc

SEQ ID NO: 20 - RNA, 10 nt, synthetic construct
  source 1..10: mol_type = other RNA
ggcagtagcc

SEQ ID NO: 21 - RNA, 56 nt, synthetic construct
  source 1..56: mol_type = other RNA
ccctgctcaagctgtgattccgagacccctgccaccactactgcattcacggggat

SEQ ID NO: 22 - RNA, 15 nt, synthetic construct
  source 1..15: mol_type = other RNA
ccctgctcaagctgt

SEQ ID NO: 23 - RNA, 15 nt, synthetic construct
  source 1..15: mol_type = other RNA
caccactactgcatt

SEQ ID NO: 24 - RNA, 76 nt, synthetic construct
  source 1..76: mol_type = other RNA
ccgaacactgagaaatagtgcactccaagaaacgtggatctccccctcatccaactccgaaagtctgaatctccca

SEQ ID NO: 25 - RNA, 15 nt, synthetic construct
  source 1..15: mol_type = other RNA
gagaaatagtgcact

SEQ ID NO: 26 - RNA, 15 nt, synthetic construct
  source 1..15: mol_type = other RNA
tccaactccgaaagt

SEQ ID NO: 27 - RNA, 15 nt, synthetic construct
  source 1..15: mol_type = other RNA
cgaaagtctgaatct

SEQ ID NO: 28 - RNA, 41 nt, synthetic construct
  source 1..41: mol_type = other RNA
gggtagagcgtctgatgaggccgaaaggccgaaaggaagtt

SEQ ID NO: 29 - RNA, 23 nt, synthetic construct
  source 1..23: mol_type = other RNA
ctgatgaggccnnnnggccgaaa

SEQ ID NO: 30 - RNA, 23 nt, synthetic construct
  source 1..23: mol_type = other RNA
ctgatgaggccagtaggccgaaa

SEQ ID NO: 31 - RNA, 23 nt, synthetic construct
  source 1..23: mol_type = other RNA
ctgatgaggcctaaaggccgaaa

SEQ ID NO: 32 - RNA, 23 nt, synthetic construct
  source 1..23: mol_type = other RNA
ctgatgaggccaaaaggccgaaa

SEQ ID NO: 33 - RNA, 23 nt, synthetic construct
  source 1..23: mol_type = other RNA
ctgatgaggccgtgaggccgaaa

SEQ ID NO: 34 - RNA, 23 nt, synthetic construct
  source 1..23: mol_type = other RNA
ctgatgaggccgagaggccgaaa

SEQ ID NO: 35 - RNA, 23 nt, synthetic construct
  source 1..23: mol_type = other RNA
ctgatgaggccgttaggccgaaa

SEQ ID NO: 36 - RNA, 23 nt, synthetic construct
  source 1..23: mol_type = other RNA
ctgatgaggccgataggccgaaa

SEQ ID NO: 37 - RNA, 23 nt, synthetic construct
  source 1..23: mol_type = other RNA
ctgatgaggccgacaggccgaaa

SEQ ID NO: 38 - RNA, 23 nt, synthetic construct
  source 1..23: mol_type = other RNA
ctgatgaggccaaagggccgaaa

SEQ ID NO: 39 - RNA, 23 nt, synthetic construct
  source 1..23: mol_type = other RNA
ctgatgaggccaacaggccgaaa

SEQ ID NO: 40 - RNA, 23 nt, synthetic construct
  source 1..23: mol_type = other RNA
ctgatgaggccattaggccgaaa

SEQ ID NO: 41 - RNA, 23 nt, synthetic construct
  source 1..23: mol_type = other RNA
ctgatgaggccaataggccgaaa

SEQ ID NO: 42 - RNA, 23 nt, synthetic construct
  source 1..23: mol_type = other RNA
ctgatgaggccagcaggccgaaa

SEQ ID NO: 43 - RNA, 23 nt, synthetic construct
  source 1..23: mol_type = other RNA
ctgatgaggccnntnggccgaaa

SEQ ID NO: 44 - RNA, 23 nt, synthetic construct
  source 1..23: mol_type = other RNA
ctgatgaggccnnnaggccgaaa

SEQ ID NO: 45 - RNA, 11 nt, synthetic construct
  source 1..11: mol_type = other RNA
gggttgagcgt

SEQ ID NO: 46 - RNA, 10 nt, synthetic construct
  source 1..10: mol_type = other RNA
gggtgaagtg

SEQ ID NO: 47 - RNA, 10 nt, synthetic construct
  source 1..10: mol_type = other RNA
gggtacgtgg

SEQ ID NO: 48 - RNA, 11 nt, synthetic construct
  source 1..11: mol_type = other RNA
gggttatgcag

SEQ ID NO: 49 - DNA, 38 bp, synthetic construct
  source 1..38: mol_type = other DNA
ccatgattacgccaagcttaatacgactcactataggg

SEQ ID NO: 50 - DNA, 38 bp, synthetic construct
  source 1..38: mol_type = other DNA
tagagcgtctgatgaggccgaaaggccgaaaggaagtt

SEQ ID NO: 51 - DNA, 38 bp, synthetic construct
  source 1..38: mol_type = other DNA
ttgagcgtctgatgaggccgaaaggccgaaaggaagtt

SEQ ID NO: 52 - DNA, 38 bp, synthetic construct
  source 1..38: mol_type = other DNA
ttgagcgtctgatcaggccgaaaggccgaaaggaagtt

SEQ ID NO: 53 - DNA, 38 bp, synthetic construct
  source 1..38: mol_type = other DNA
tggagcgtctgatgaggccgaaaggccgaaaggaagtt

SEQ ID NO: 54 - DNA, 35 bp, synthetic construct
  source 1..35: mol_type = other DNA
ttgagcgtctgatgaggccnggccgaaaggaagtt

SEQ ID NO: 55 - DNA, 25 bp, synthetic construct
  source 1..25: mol_type = other DNA
ccatgattacgccaagcttaatacg

SEQ ID NO: 56 - DNA, 22 bp, synthetic construct
  source 1..22: mol_type = other DNA
aacttcctttcggcctttcggc

SEQ ID NO: 57 - DNA, 22 bp, synthetic construct
  source 1..22: mol_type = other DNA
aacttcctttcggccnnnnggc

SEQ ID NO: 58 - DNA, 36 bp, synthetic construct
  source 1..36: mol_type = other DNA
ccatgattacgccaagcttaatacgactcactatag

SEQ ID NO: 59 - DNA, 38 bp, synthetic construct
  source 1..38: mol_type = other DNA
gcgatgacctgatgaggccgaaaggccgaaacgttccc

SEQ ID NO: 60 - DNA, 38 bp, synthetic construct
  source 1..38: mol_type = other DNA
gcgatgacctgatcaggccgaaaggccgaaacgttccc

SEQ ID NO: 61 - DNA, 21 bp, synthetic construct
  source 1..21: mol_type = other DNA
gggaacgtttcggcctttcgg

SEQ ID NO: 62 - DNA, 60 bp, synthetic construct
  source 1..60: mol_type = other DNA
ccatgattacgccaagcttaatacgactcactatagggtttagtgaaccgtcagatccgc

SEQ ID NO: 63 - DNA, 40 bp, synthetic construct
  source 1..40: mol_type = other DNA
agaatgcagtgaaaaaaatgctttatttgtgaaatttgtg

SEQ ID NO: 64 - DNA, 60 bp, synthetic construct
  source 1..60: mol_type = other DNA
ccatgattacgccaagcttaatacgactcactatagggagagtcatccagctggagccct

SEQ ID NO: 65 - DNA, 26 bp, synthetic construct
  source 1..26: mol_type = other DNA
aagttgctcatgggcttacacaccac

SEQ ID NO: 66 - DNA, 95 bp, synthetic construct
  source 1..95: mol_type = other DNA
ggtttagtgaaccgtcagatccgctagcgtgctgggcttccccatcaacttcctcacgctctacgtcaccgtccagcacaccggtcgccaccatg

SEQ ID NO: 67 - DNA, 24 bp, synthetic construct
  source 1..24: mol_type = other DNA
ggtttagtgaaccgtcagatccgc

SEQ ID NO: 68 - DNA, 19 bp, synthetic construct
  source 1..19: mol_type = other DNA
catggtggcgaccggtgtg

SEQ ID NO: 69 - DNA, 199 bp, synthetic construct
  source 1..199: mol_type = other DNA
agtccggactcagatcttaagtgctgggcttccccatcaacttcctcacgctctacgtcaccgtccagcaccgagctcaagcttgtgctgggcttccccatcaacttcctcacgctctacgtcaccgtccagcaccgaattctgcagtgtgctgggcttccccatcaacttcctcacgctctacgtcaccgtccagcac

SEQ ID NO: 70 - DNA, 38 bp, synthetic construct
  source 1..38: mol_type = other DNA
gcggcttgtacaagtccggactcagatcttaagtgctg

SEQ ID NO: 71 - DNA, 34 bp, synthetic construct
  source 1..34: mol_type = other DNA
gcggctggatccgtgctggacggtgacgtagagc

SEQ ID NO: 72 - RNA, 15 nt, synthetic construct
  source 1..15: mol_type = other RNA
cgtggatctccccct

SEQ ID NO: 73 - DNA, 71 bp, synthetic construct
  source 1..71: mol_type = other DNA
aaaaaaaaaaaaaaagggttgagcgtctgatgaagccagtaggccgaaaggaagttaaaaaaaaaaaaaaa

SEQ ID NO: 74 - DNA, 44 bp, synthetic construct
  source 1..44: mol_type = other DNA
gcggctggatccaaaaaaaaaaaaaaagggttgagcgtctgatg

SEQ ID NO: 75 - DNA, 48 bp, synthetic construct
  source 1..48: mol_type = other DNA
gcggctggatcctttttttttttttttaacttcctttcggcctactgg

SEQ ID NO: 76 - DNA, 646 bp, synthetic construct
  source 1..646: mol_type = other DNA
tgtacaagtccggactcagatcttaagtgctgggcttccccatcaacttcctcacgctctacgtcaccgtccagcaccgagctcaagcttgtgctgggcttccccatcaacttcctcacgctctacgtcaccgtccagcaccgaattctgcagtgtgctgggcttccccatcaacttcctcacgctctacgtcaccgtccagcacggatccaaaaaaaaaaaaaaagggttgagcgtctgatgaggccagtaggccgaaaggaagttaaaaaaaaaaaaaaggatccaaaaaaaaaaaaaaaagggttgagcgtctgatgaggccagtaggccgaaaggaagttaaaaaaaaaaaaaaaggatccaaaaaaaaaaaaaaaagggttgagcgtctgatgaggccagtaggccgaaaggaagttaaaaaaaaaaaaaaaggatccaaaaaaaaaaaaaaagggttgagcgtctgatgaggccagtaggccgaaaggaagttaaaaaaaaaaaaaaaggatccaccggatctagataactgatcataatcagccataccacatttgtagaggttttacttgctttaaaaaacctcccacacctccccctgaacctgaaacataaaatgaatgcaattgttgttgttaac

SEQ ID NO: 77 - DNA, 28 bp, synthetic construct
  source 1..28: mol_type = other DNA
gggttgagcgtctgatcaggccagtagg

SEQ ID NO: 78 - DNA, 28 bp, synthetic construct
  source 1..28: mol_type = other DNA
cctactggcctgatcagacgctcaaccc

SEQ ID NO: 79 - DNA, 22 bp, synthetic construct
  source 1..22: mol_type = other DNA
gactacttgaagctgtccttcc

SEQ ID NO: 80 - DNA, 15 bp, synthetic construct
  source 1..15: mol_type = other DNA
gcgcgtgatgaactt

SEQ ID NO: 81 - DNA, 20 bp, synthetic construct
  source 1..20: mol_type = other DNA
cgcagcttcaccttgtagat

SEQ ID NO: 82 - DNA, 20 bp, synthetic construct
  source 1..20: mol_type = other DNA
gagatcaagcagaggctgaa

SEQ ID NO: 83 - DNA, 15 bp, synthetic construct
  source 1..15: mol_type = other DNA
acaaggccaagaagc

SEQ ID NO: 84 - DNA, 21 bp, synthetic construct
  source 1..21: mol_type = other DNA
acttgatgtcgacgttgtagg

SEQ ID NO: 85 - DNA, 23 bp, synthetic construct
  source 1..23: mol_type = other DNA
aatttggaggcttctttgccacc

SEQ ID NO: 86 - DNA, 25 bp, synthetic construct
  source 1..25: mol_type = other DNA
aaattgccctgtggtccttggtggt

SEQ ID NO: 87 - DNA, 24 bp, synthetic construct
  source 1..24: mol_type = other DNA
agttgctcatgggcttacacacca

SEQ ID NO: 88 - RNA, 18 nt, synthetic construct
  source 1..18: mol_type = other RNA
agggaacgtcgtcgtcgc

SEQ ID NO: 89 - RNA, 41 nt, synthetic construct
  source 1..41: mol_type = other RNA
gggttgagcgtctgatgaggccgaaaggccgaaaggaagtt

SEQ ID NO: 90 - RNA, 41 nt, synthetic construct
  source 1..41: mol_type = other RNA
gggttgagcgtctgatgrggccagtaggccgaaaggaagtt

SEQ ID NO: 91 - RNA, 41 nt, synthetic construct; identical to SEQ ID NO: 1
  source 1..41: mol_type = other RNA

SEQ ID NO: 92 - RNA, 40 nt, synthetic construct
  source 1..40: mol_type = other RNA
gggttgagcgtcgatgaggccagtaggccgaaaggaagtt

SEQ ID NO: 93 - RNA, 41 nt, synthetic construct; identical to SEQ ID NO: 1
  source 1..41: mol_type = other RNA

SEQ ID NO: 94 - RNA, 41 nt, synthetic construct; identical to SEQ ID NO: 1
  source 1..41: mol_type = other RNA

SEQ ID NO: 95 - RNA, 41 nt, synthetic construct; identical to SEQ ID NO: 1
  source 1..41: mol_type = other RNA

SEQ ID NO: 96 - RNA, 40 nt, synthetic construct; identical to SEQ ID NO: 92
  source 1..40: mol_type = other RNA

SEQ ID NO: 97 - RNA, 40 nt, synthetic construct; identical to SEQ ID NO: 92
  source 1..40: mol_type = other RNA

SEQ ID NO: 98 - RNA, 41 nt, synthetic construct; identical to SEQ ID NO: 89
  source 1..41: mol_type = other RNA

SEQ ID NO: 99 - RNA, 41 nt, synthetic construct
  source 1..41: mol_type = other RNA
gggtggagcgtctgatgaggccgaaaggccgaaaggaagtt

SEQ ID NO: 100 - RNA, 41 nt, synthetic construct
  source 1..41: mol_type = other RNA
gggtagagcgtctgatgaggcccttgggccgaaaggaagtt

SEQ ID NO: 101 - RNA, 41 nt, synthetic construct
  source 1..41: mol_type = other RNA
gggtagagcgtctgatgaggccttcgggccgaaaggaagtt

SEQ ID NO: 102 - RNA, 41 nt, synthetic construct; identical to SEQ ID NO: 100
  source 1..41: mol_type = other RNA

SEQ ID NO: 103 - RNA, 41 nt, synthetic construct; identical to SEQ ID NO: 7
  source 1..41: mol_type = other RNA

SEQ ID NO: 104 - RNA, 41 nt, synthetic construct
  source 1..41: mol_type = other RNA
gggttgagcgtctgatgaggcccaaaggccgaaaggaagtt

SEQ ID NO: 105 - RNA, 41 nt, synthetic construct; identical to SEQ ID NO: 5
  source 1..41: mol_type = other RNA

SEQ ID NO: 106 - RNA, 41 nt, synthetic construct; identical to SEQ ID NO: 6
  source 1..41: mol_type = other RNA

SEQ ID NO: 107 - RNA, 41 nt, synthetic construct
  source 1..41: mol_type = other RNA
gggttgagcgtctgatgaggccggaaggccgaaaggaagtt

SEQ ID NO: 108 - RNA, 41 nt, synthetic construct; identical to SEQ ID NO: 9
  source 1..41: mol_type = other RNA

SEQ ID NO: 109 - RNA, 41 nt, synthetic construct
  source 1..41: mol_type = other RNA
gggttgagcgtctgatgaggccgcaaggccgaaaggaagtt

SEQ ID NO: 110 - RNA, 41 nt, synthetic construct; identical to SEQ ID NO: 8
  source 1..41: mol_type = other RNA

SEQ ID NO: 111 - RNA, 41 nt, synthetic construct; identical to SEQ ID NO: 10
  source 1..41: mol_type = other RNA

SEQ ID NO: 112 - RNA, 41 nt, synthetic construct; identical to SEQ ID NO: 11
  source 1..41: mol_type = other RNA

SEQ ID NO: 113 - RNA, 41 nt, synthetic construct
  source 1..41: mol_type = other RNA
gggttgagcgtctgatgaggccgaagggccgaaaggaagtt

SEQ ID NO: 114 - RNA, 41 nt, synthetic construct
  source 1..41: mol_type = other RNA
gggttgagcgtctgatgaggccgaacggccgaaaggaagtt

SEQ ID NO: 115 - RNA, 41 nt, synthetic construct
  source 1..41: mol_type = other RNA
gggttgagcgtctgatgaggccgaatggccgaaaggaagtt

SEQ ID NO: 116 - RNA, 41 nt, synthetic construct; identical to SEQ ID NO: 12
  source 1..41: mol_type = other RNA

SEQ ID NO: 117 - RNA, 41 nt, synthetic construct; identical to SEQ ID NO: 13
  source 1..41: mol_type = other RNA

SEQ ID NO: 118 - RNA, 41 nt, synthetic construct; identical to SEQ ID NO: 14
  source 1..41: mol_type = other RNA

SEQ ID NO: 119 - RNA, 41 nt, synthetic construct; identical to SEQ ID NO: 15
  source 1..41: mol_type = other RNA

SEQ ID NO: 120 - RNA, 41 nt, synthetic construct; identical to SEQ ID NO: 16
  source 1..41: mol_type = other RNA

SEQ ID NO: 121 - RNA, 41 nt, synthetic construct
  source 1..41: mol_type = other RNA
gggttgagcgtctgatgaggccttcgggccgaaaggaagtt

SEQ ID NO: 122 - RNA, 41 nt, synthetic construct; identical to SEQ ID NO: 121
  source 1..41: mol_type = other RNA

(19) United States
(12) Patent Application Publication
(10) Pub. No.: US 2025/0283077 A1
(43) Pub. Date: Sep. 11, 2025

(54) ENHANCED HAMMERHEAD RIBOZYMES AND METHODS OF USE

(71) Applicants: The United States Government as represented by the Department of Veterans Affairs, Washington, DC (US); UNIVERSITY AT BUFFALO, Buffalo, NY (US)

(72) Inventors: John M. Sullivan, Buffalo, NY (US); Jason M. Myers, Buffalo, NY (US)

(21) Appl. No.: 18/858,977

(22) PCT Filed: Apr. 21, 2023
(86) PCT No.: PCT/US2023/066080

§ 371 Date: Oct. 22, 2024

Related U.S. Application Data
(60) Provisional application No. 63/333,897, filed on Apr. 22, 2022.

(51) Int. Cl. C12N 15/113

(52) U.S. Cl. CPC C12N 15/113; C12N 2310/121; C12N 2310/321; C12N 2310/3231; C12N 2310/333; C12N 2310/3341; C12N 2310/335; C12N 2310/531; C12N 2320/32

(57) ABSTRACT

Disclosed herein, are hammerhead ribozymes that bind to a target mRNA comprising a NUH ribozyme cleavage site that have enhanced turnover rates. Also described herein, are methods of administering hammerhead ribozymes that bind to a target mRNA comprising a NUH ribozyme cleavage site to treat eye diseases.

### CROSS REFERENCE TO RELATED APPLICATIONS

This application claims the benefit of U.S. Provisional Application No. 63/333,897, filed Apr. 22, 2022. The content of this earlier filed application is hereby incorporated by reference herein in its entirety.

### STATEMENT REGARDING FEDERALLY FUNDED RESEARCH

This invention was made with government support under grant number 1I01BX000669 awarded by the United States Department of Veterans Affairs and grant number EY013433 awarded by the National Institutes of Health. The government has certain rights in the invention.

### INCORPORATION OF THE SEQUENCE LISTING

The present application contains a sequence listing that is submitted concurrent with the filing of this application, containing the file name “37759_0459P1_Sequence_Listing.xml” which is 114,688 bytes in size, created on Apr. 21, 2023, and is herein incorporated by reference in its entirety.

### SUMMARY

Disclosed herein are hammerhead ribozymes comprising a stem II region, and a stem terminator loop, wherein the stem terminator loop comprises the nucleic sequence of AGUA, UAAA, AAAA, GUGA, GAGA, GAUA, GUUA, GACA, AAAG, AACA, AUUA, AAUA, or AGCA.

Disclosed herein are hammerhead ribozymes comprising an enzyme core, a stem II region, a stem terminator loop, a first flanking region at the 5′ end and a second flanking region at the 3′ end, wherein the first flanking region comprises a uracil at position 7.

Disclosed herein are hammerhead ribozymes comprising a nucleic acid sequence of CUGAUGA GGCC X₁X₂X₃X₄ GGCC GAA (SEQ ID NO: 29), wherein X₁X₂X₃X₄ of SEQ ID NO: 29 are AGUA, UAAA, AAAA, GUGA, GAGA, GUUA, GAUA, GACA, AAAG, AACA, AUUA, AAUA, or AGCA, and wherein the nucleic acid sequence has an enhanced turnover rate compared to a wild-type hammerhead ribozyme.

Other features and advantages of the present compositions and methods are illustrated in the description below, the drawings, and the claims.

### BRIEF DESCRIPTION OF THE DRAWINGS

FIGS. 1A-D show EhhRz schematic and quantitative high-throughput fluorescence assay for hhRz kinetics. FIG. 1A Shows a 2D schematic of an optimized EhhRz (GGGU—U/A—GAGCGU-CUGAUGAGGCCAGUAGGCCGAAAGGAAGUU; SEQ ID NO: 1, with the U; and SEQ ID NO: 2 with the A) targeting the 266 CUCd cleavage site in human rhodopsin mRNA (hRHO). The short model (15-mer) substrate is shown in green (ACUUCCUCIACGCUCU; SEQ ID NO: 17). The centered NUH↓ cleavage site (here CUC↓) is labeled for an EhhRz with 7 nt antisense flanks (labeled in black); note that C17 of the substrate does not base pair with the hhRz. The location of the substrate U1.7 residue is shown. The location of the A2.7 residue of the EhhRz or its alternative U2.7 residue is shown; this constitutes the location in the EhhRz of what we call the A7U mutation. The enzyme core (red) is numbered with the classical hhRz numbering system (Hertel et al., 1992). To inactivate the EhhRz a G8C mutation is made in the enzyme core. The 4 bp Stem-II region (blue) is shown. The Stem-II capping tetraloop (violet) shows an optimized AGUA for the L2.1 to L2.4 residues. FIG. 1B, a shows the short RNA substrate has a fluorophore (here, FAM) at the 5′ end and a quencher (here, black hole quencher 1 [BHQ-1]) at the 3′ end. The short substrate is drawn as a random coil (slight arc) without significant secondary structure. The quencher and the fluorophore are within Förster distance (1/r6 dependence, 10-100 Å with R0 [50%] distance particular to each pair) such that the fluorescence energy emitted from FAM is absorbed (quenched) by BHQ-1. FIG. 1B, b shows that upon annealing of the hhRz, the random coil of the substrate is disrupted with further separation from the quencher, thus increasing fluorescence emission. FIG. 1B, c shows that upon cleavage of the substrate by the hhRz at the NUH↓ site (here CUC↓), release of products, and diffusion, the fluorophore becomes well separated from the quencher, and emission is significantly enhanced (shown in schematic as increased emission arrow). This generates a real-time quantitative assay of enzymatic turnover (binding, cleavage, product release→next cycle), which can be measured in a quantitative PCR machine. FIG. 1C shows the time-dependent accumulation of FAM fluorescence in hhRz reactions. The reactions were conducted with substrate in 10-fold molar excess over the hhRz (1 μM and 0.1 μM, respectively), with 0.5 mM Mg²⁺ at 37° C. Reactions were initiated by adding the hhRz to the substrate equilibrated in buffer (10 mM Tris-HCL, pH 7.5). The initial slopes of the time-dependent FAM fluorescence changes were used in subsequent analyses. Results were scaled by the FAM units per nanomolar (nM) for the product (without BHQ1). WT (GAAA) 266 is the first EhhRz identified and showed a significant positive slope well fit by a linear function. A7U (GAAA) is the WT 266 EhhRz with the upstream “A7” residue of the antisense flank mutated to U (to prevent base pairing with substrate at U7) and showed a stronger positive slope, also well fit by a linear function. A7U (AGUA) is the A7U 266 EhhRz with the stem II tetraloop changed from GAAA to AGUA and is the fastest EhhRz identified to date; it ultimately saturated the silicon photodiode detector in the qPCR machine. The substrate alone showed no definitive positive rate. Mutation of the catalytic core (G5C, G8C, G12C) (Ruffner et al., 1990) in A7U (GAAA) EhhRz also obviated the turnover activity of the enzyme. HH16 is a well-studied mhhRz (Hertel et al., 1994, 1996) and showed a slight fluorescence change during the 5-min recording cycle. Note that the active EhhRzs showed a positive fluorescence shift evident above that for the substrate alone at time zero; this finding likely relates to the binding of the substrate by the enzyme. FIG. 1D shows the gel-based analysis corresponding to the optical assay. SYBR Gold staining for gels with the 15-mer substrate (FIG. 1D, a) and the 14-mer substrate (FIG. 1D, b) showed the definitive formation of product(s) of expected size during the 5-min reaction with substrate in excess over EhhRz (10:1 molar ratio) and 0.5 mM Mg²⁺. (FIG. 1D, c) Conversion of substrate to product is apparent in the gel imaged for FAM fluorescence. Catalytic core mutation in the EhhRzs completely suppressed cleavage, as expected. Black arrows, EhhRz; blue arrows, FAM-labeled substrate; red arrows, FAM-labeled product.

FIGS. 2A-F show 266 hRHO substrate and product fluorescence-concentration relationships. (Top panel) Relationship of 15-mer 266 substrate RNA concentration to its fluorescence. The substrate has a 5′ FAM fluor and a 3′ BHQ1 quencher. FIG. 2A shows the concentration was varied from 50 nM to 5000 nM and fluorescence measured over a 5 minute period. Fluorescence emissions were stable over time at all concentrations. FIG. 2B shows the relationship of fluorescence to concentration was well-fit by a linear relationship (R²=0.99602) with a slope of 0.25836 FAM units/nM. (Middle panel). Relationship of 14-mer 266 Substrate RNA concentration to its fluorescence. The substrate has a 5′ FAM fluor and a 3′ BHQ1 quencher. FIG. 2C shows the concentration was varied from 50 nM to 2,500 nM and fluorescence measured over a 5 minute period. Fluorescence emissions were stable over time at all concentrations. FIG. 2D show the relationship of fluorescence to concentration was well-fit by a linear relationship (R²=0.99235) with a slope of 0.47175 FAM units/nM. (Bottom Panel). Relationship of 8-mer hRHO 266 Product RNA concentration to its fluorescence. The product RNA has a 5′ FAM fluor and no BHQ1 quencher at the 3′ end. The 8-mer product is the same for both the 15-mer and 14-mer substrates. The concentration was varied from 7.81 nM to 600 nM and fluorescence measured over a 5 minute period (FIG. 2E). Fluorescence emissions were stable over time at the concentrations tested. FIG. 2F shows the relationship of fluorescence to concentration was well-fit by a linear relationship (R²=0.99788) with a slope of 2.63555 FAM units/nM. The results of separate experiments (n=3) to measure the 8-mer 266 product specific fluorescence found Mean±SD at 3.11455±0.435 FAM Units/nM; the scale factor of 3.11 FAM Units/nM was used to scale the quantitative fluorescence measures in these experiments.

FIGS. 3A-B show HH16 product fluorescence-concentration relationships. Relationship of 10-mer HH16 product RNA concentration to its fluorescence. The HH16 product has a 5′ FAM fluor and no BHQ1 quencher at the 3′ end. The concentration was varied from 15.625 nM to 1000 nM and fluorescence measured over a 5 minute period (FIG. 3A). Fluorescence emissions were stable over time at the concentrations tested. FIG. 3B shows the relationship of fluorescence to concentration was well-fit by a linear relationship (R²=0.99125) with a slope of 0.97838 FAM units/nM.

FIGS. 4A-F show the substrate structure RNA folding analysis for target RNAs. RNA Structure was used to fold the substrate RNAs for hRHO 266 and HH16 into secondary structures (FIGS. 4A, 4D). There is no predicted secondary structure for 266 hRHO substrate (here 15-mer) and predicted secondary structure for HH16 substrate (18-mer) involving 4 bps. RNA Composer was used to examine possible tertiary structures for the 266 and HH16 substrates. The hRHO 15-mer substrate yields no apparent tertiary structures (FIGS. 4B, 4C). The HH16 substrate yields tertiary structures shows from two perspectives (FIGS. 4E, 4F).

FIGS. 5A-B show the comparison of kinetic impacts of hhRz upstream A7 replacements and stem II tetraloop replacements. Means and standard deviations are shown in the graphs. FIG. 5A shows the data regarding the upstream A7 replacements. The substrate U7 residue in the wild type (WT) (GAAA) EhhRz is in a Watson-Crick base pair with “A7” in the upstream antisense flank of the EhhRz. The nomenclature used indicates that the A7 residue of the hhRz is the nucleotide in the upstream antisense flank that pairs with the U7 in the substrate RNA. U7 is expected to interact with the fourth nucleotide (underlined) of the GAAA tetraloop triplet of the hhRz (O'Rourke et al., 2015) via Hoogsteen face interactions. The rates were scaled by product fluorescence per nM (3.11 FAM units/nM). The A7 residue was mutated in the hhRz to U (A7U) to obviate formation of the Watson-Crick base pair with the substrate to relieve the constraint that could influence interactions with the tetraloop. The hhRz A7 residue was also mutated to G (A7G), which can still interact with substrate U7 by Watson-Crick pairing (G:U). Sample numbers were as follows: WT 266, n=36; A7U 266, n=52; A7G 266, n=23. Kolmogorov-Smirnov tests for normality indicated that the samples were drawn from a normally distributed populations. Homogeneity of variance test (Levene's absolute) showed significant differences in population variance (p=1.69E-11). By parametric one-way ANOVA the sample means were found to be different (p=3.86E-41). Post-hoc testing by both parametric and nonparametric analyses indicated that the WT differs (*p<0.05) from A7U (p values: Bonferroni, 1.59E-33; Tukey, <0.01; Fisher, 5.31E-34; Bonferroni-Holm, 5.31E-34) and A7G (p values: Bonferroni, 0.0064; Tukey, 0.006; Fisher, 0.002; Bonferroni-Holm, 7.21E-36), and that the A7U and A7G variants also have different means (#p<0.05) (p values: Bonferroni, 2.16E-35; Tukey, <0.01; Fisher, 7.21E-36; Bonferroni-Holm, 7.21E-36). By nonparametric Kruskal-Wallis ANOVA the sample means were found to be different (Chi-square p=1.33E-20) and post-hoc Dunn's Test found WT different from A7U (p=2.64E-10) and A7G (p=0.007) and A7U different from A7G (p=2.12E-18). FIG. 5B shows the tetraloop replacements. The activity of WT (GAAA) EhhRz was compared to that of the WT (UUCG) and WT (CUUG) enzymes with stem II tetraloop variations. GAAA is from the class of GNRA tetraloops, and UUCG is from the class of UNCG tetraloops. Sample numbers were as follows: WT 266(GAAA), n=36; WT 266(UUCG), n=8; WT 266(CUUG), n=8. Kolmogorov-Smimov tests for normality indicated that the samples were drawn from normally distributed populations. Homogeneity of variance test (Levene's absolute) showed significant differences in population variance (p=4.58E-6). By parametric one-way ANOVA the sample means were found to be different (p=2.27E-30). Post-hoc testing by both parametric and nonparametric analyses indicated that the WT 266(GAAA) differs (*p<0.05) from WT 266(UUCG) (p values: Bonferroni, 2.08E-26; Tukey, <0.01; Fisher, 6.93E-27; Bonferroni-Holm, 6.93E-27) and from WT 266(CUUG) (p values: Bonferroni, 7.55E-25; Tukey, <0.01; Fisher, 2.52E-25; Bonferroni-Holm, 2.52E-25); the WT 266(UUCG) and WT 266(CUUG) means were not different (p values: Bonferroni, 0.572; Tukey, 0.38; Fisher, 0.19; Bonferroni-Holm, 0.19). By nonparametric Kruskal-Wallis ANOVA the sample means were found to be different (Chi-square p=4.71E-8) and post-hoc Dunn's Test found WT 266(GAAA) different from WT 266(UUCG) (p=1.22E-6) and WT 266(CUUG) (p=6.09E-4) but WT(UUCG) not different from WT(CUUG) (p=0.87).

FIGS. 6A-D show the effects of various hhRz stem-loop II tetraloop variants on catalytic activity with and without U7 in substrate. The initial rates of reaction were measured at an enzyme concentration of 100 nM and substrate concentration of 1 M (10:1) at 0.5 mM Mg²⁺ and 37° C. The rates were scaled by mean product fluorescence per nanomolar (3.11 FAM units/nM). Tetraloops were varied on the 266 A7U(GAAA) EhhRz (“WT”). FIG. 6A shows the 15-mer substrate RNA. Kolmogorov-Smirnov tests for normality indicated that the samples were drawn from normally distributed populations. Homogeneity of variance test (Levene's absolute) showed significant differences in population variance (p=3.05E-13). The 266 A7U EhhRz with a WT stem II tetraloop (GAAA) showed strong activity (96.84±18.21 min⁻¹). When GAAA was replaced with 22 other tetraloops, the turnover activity was suppressed or enhanced relative to that with the WT tetraloop. By parametric one-way ANOVA the sample means were found to be different (p=1.22E-114). Post-hoc Bonferroni tests (*p<0.05) indicated means significantly different from A7U (GAAA) (“WT”). The EhhRz with greatest turnover rate relative to GAAA had an AGUA tetraloop (169.32±19.86 min⁻¹) (blue asterisk). Post-hoc Bonferroni tests showed constructs with turnover rate significantly different from AGUA (#p<0.05). Constructs with means not significantly different from AGUA were AAAA, GAUA, AUUA, AAUA, and AGCA. By nonparametric Kruskal-Wallis ANOVA the sample means were found to be different (Chi-square p=3.56E-50). Post-hoc Dunn's Test found the following constructs to have significant differences of mean vs A7U(GAAA) (“WT”): AAAA (p=0.016), GUUA (p=3.80E-6), AUUA (p=7.05E-13), AAUA (p=3.97E-9), and AGUA (p=3.52E-16). FIG. 6B shows the 14-mer substrate RNA (no U7 residue). Activity of the WT enzyme remained high (23.42±2.27 min⁻¹) but varied with the different stem II tetraloop compositions. Kolmogorov-Smimov tests for normality indicated that all the samples were drawn from normally distributed populations. Homogeneity of variance test (Levene's absolute) showed significant differences in population variance (p=2.00E-15). By parametric one-way ANOVA the sample means were found to be different (p=2.35E-120). Post-hoc Bonferroni tests (*p<0.05) indicated means significantly different from A7U(GAAA) (“WT”). The EhhRz with greatest turnover rate relative to GAAA also had an AGUA tetraloop (95.15±10.89 min⁻¹) (blue asterisk). Post-hoc Bonferroni tests showed constructs with turnover rate significantly different from AGUA (^{#}p<0.05). Constructs with means not significantly different from AGUA were only AAAG. By nonparametric Kruskal-Wallis ANOVA the sample means were found to be different (Chi-square p=3.73E-43). Post-hoc Dunn's Test found the following constructs to have significant differences of mean vs A7U(GAAA) (“WT”): AAAA (p=0.003), GAUA (p=0.026), GAAG (p=0.032), GAAU (p=0.006), AAAG (p=1.54E-4), AUUA (p=0.0095), AAUA (p=1.00E-4), and AGUA (p=3.16E-5). FIG. 6C shows the ordered comparison of mean rates for selected EhhRzs against 14-mer and 15-mer substrates. FIG. 6D shows the boxcar comparison of selected EhhRzs against 14-mer and 15-mer substrates shown in (FIG. 6C). The box outlines embrace the 75^{th} and 25th percentiles, and enclose the means (□), and median (-); the maximum and minimum values (x) and the 1^{st} and 99^{th} percentiles (-) are shown. The numbers (1-5) above the boxcars refer to apparent quantized presentation of outcomes into groups.

FIGS. 7A-F show the RNA folding analysis to investigate the relationship of stem II tetraloop accessibility and cleavage activity. The set of hhRz sequence variants used in the Stem II tetraloop sampling were evaluated by 2D RNA folding analysis using Sfold and Sma. The sequences were identical except for the four nucleotides of the tetraloop in order to evaluate the effect of the tetraloop of accessibility of the loop and the relationship of accessibility to experimental enzymatic activity. An identical ensemble centroid structure (FIG. 7A) was found for 21 of the 23 sampled sequences and shows a partial interaction of the antisense flanks and enzyme core with an expected four base pair Stem II interaction. The ensemble centroids for the EhhRzs with tetraloops AACA (FIG. 7B) and AGCA (FIG. 7C) show an open antisense flank structures and a lack of the last G:::C base pair of Stem II. The high probability cluster centroids showed the structure found for the dominant ensemble centroid (FIG. 7A). The number of cluster centroids varied. The free energies of the ensemble centroids, the two dominant cluster centroids, the residual probability of additional clusters, and the experimental enzymatic activity of each of the hhRzs tested are shown (FIG. 7D). The Sfold accessibility probability map was generated for each of the 23 sampled hhRzs and plotted in an overlay (FIG. 7E) and showed strong accessibility at the region of the tetraloop sequence. This includes the EhhRzs with AACA and AGCA tetraloops, which are skewed in the accessibility because of the 6 nt tetraloops predicted. A comparison of the mean (+/−SD) experimental enzyme activity and the mean access probability of the tetraloop sequence within the EhhRzs shows no strong functional relationship (FIG. 7F), which indicates that it is the chemistry of the tetraloop and not its accessibility which dominates the variability of the enzyme activity.

FIGS. 8A-I show Michaelis-Menten analysis of EhhRzs against the 15-mer substrate. The turnover rates for 1 μM substrate were evaluated for various concentrations of the three EhhRzs: WT(GAAA) 266 EhhRz (FIG. 8A), A7U(GAAA) 266 EhhRz (FIG. 8D), and A7U(AGUA) 266 EhhRz (FIG. 8G). The rates were scaled by product fluorescence per nanomolar (3.11 FAM Units/nM). Substrate turnover rates increased linearly over this range of enzyme concentrations for all three constructs, and the estimated turnover rates were greater than 1×10⁸ min⁻¹M⁻¹ with a rank order of A7U(AGUA)>A7U(GAAA)>WT(GAAA). Classic Michaelis-Menten analyses were conducted measuring initial rates with various substrate concentrations and the enzyme concentration fixed at 50 nM: WT(GAAA) 266 EhhRz (FIG. 8B), A7U(GAAA) 266 EhhRz (FIG. 8E), and A7U(AGUA) 266 EhhRz (FIG. 8H). The turnover titrations are well fit by the Michaelis-Menten hyperbolic function using nonlinear curve fitting. Vₘₐₓ and Kₘ were parameters extracted from the fittings (Table 3). Rankings of Vₘₐₓ and Kₘ values and enzymatic efficiency (Vₘₐₓ/Kₘ) (turnover) matched that for enzyme excess experiments: A7U(AGUA)>A7U(GAAA)>WT(GAAA). Eadie-Hofstee linear transforms and fitting of the Michaelis-Menten data corroborate the findings of the nonlinear curve fitting for WT(GAAA) 266 EhhRz (FIG. 8C), A7U(GAAA) 266 EhhRz (FIG. 8F), and A7U(AGUA) 266 EhhRz (FIG. 8I).

FIGS. 9A-F show that Mg²⁺ dependence of EhhRzs. Mg²⁺ concentrations were varied in reactions with 100 nM EhhRz and 1 μM 15-mer substrate at 37° C. and pH 7.5 (10 mM Tris-HCl). The rates were scaled by product fluorescence per nanomolar (3.11 FAM Units/nM). The Mg²⁺ sensitivity of WT(GAAA) 266 EhhRz was well fit by a double Boltzmann function (R2=0.99783) (FIG. 9A) and a Hill function (R2=0.98254) (FIG. 9B). The Mg²⁺ sensitivity of A7U(GAAA) EhhRz was well fit by a double Boltzmann function (R2=0.9955) (FIG. 9C) and a Hill function (R2=0.99384) (FIG. 9D). The Mg²⁺ sensitivity of A7U(AGUA) EhhRz was well fit by a double Boltzmann function (R2=0.9987) (FIG. 9E) and a Hill function (R2=0.99685) (FIG. 9F). Nonlinear fitting parameters for the three EhhRzs are shown in Table 3.

FIG. 10 shows single-turnover conditions in which the substrate is pre-annealed to the EhhRz. Optical assay in which 10 M EhhRz was mixed with 1 μM 15-mer substrate in 10 mM Tris-HCl (pH 7.5). The solution was heated to 95° C. for 2 min and 65° C. for 2 min and then cooled to room temperature. Reaction was catalyzed by adding Mg²⁺ to final concentration of 0.5 mM, and reaction tubes were placed immediately (within 30 s) in the real-time PCR machine. WT(GAAA) EhhRz showed a measurable nonhyperbolic (linear) rate under these conditions (initial rate was fitted over first 132 sec); two points were measurable for the A7U(GAAA) to obtain an estimated rate, and the A7U(AGUA) enzyme fully cleaved the substrate by the first time point. Non-cleavable substrates with CUG motifs showed lower levels of initial fluorescence and no evidence of cleavage.

FIGS. 11A-F show full length target and in cellulo analysis of EhhRz function. FIG. 11A shows mCherry fusion RNA reporter. Fifty-one nt hRHO 266 CUC targets (“lollypops”) were embedded in the 5′ and 3′ UTR of the mCherry reporter. One 51 nt hRHO lollypop was inserted in the 5′UTR of mCherry, and three 51 nt hRHO lollypops in the 3′ UTR of mCherry. 1, 2, or 4 EhhRzs (A7U AGUA 266 EhhRz) were appended downstream of the last hRHO target element. FIG. 11B show 51 nt hRHO target sequence. 2D RNA folding algorithms predict a stable stem with a 33nt single stranded loop (266 CUC↓, red arrow). FIG. 1C shows in vitro transcription of the mCherry fusion RNA reporter. EhhRzs embedded in the mCherry reporter are liberated upon Cis cleavage (bracketed products). Combinatorial banding pattern (squares) is proportional to the number of EhhRzs embedded with 4 EhhRzs resulting in almost all of the full length reporter cleaved (green arrow). FIG. 11D shows cis liberated EhhRzs cleavage activity in trans on a structured 532 nt hRHO mRNA containing a native 266 CUCA site. 532 nt hRHO mRNA fragment with endogenous 266 CUC↓ (Lane 2). Increasing number of Cis EhhRzs in the fusion RNA reporter generate increasing amounts of the endogenous 266 cleavage product (lanes: 4, 6, and 8, green arrow). Converting the CUC↓ cleavage site to non-cleaving CUG demonstrates no cleavage products (lanes: 3, 5, and 7). FIG. 11E shows the enzyme core mutation G8C abolishes catalytic activity. G8C mutations on 4 EhhRzs in Cis with the mCherry fusion RNA reporter abolish catalytic activity (lane 2) vs the catalytically active Cis EhhRzs (lane 3). FIG. 11F shows in vivo knockdown of fusion RNA reporter by RT-PCR. Pooled mean from inactive and active EhhRzs Ct values and relative fold suppression (2^{-ΔΔCt}) with the active EhhRz (* indicates p<0.05). FIG. 11G shows in vivo mCherry reporter knockdown. In vivo functional assay measuring 80% suppression of mCherry fluorescence with the active EhhRzs relative to the catalytically inactive EhhRzs (* indicates p<0.05).

FIG. 12 shows the kinetic parameters calculated for Stage-Zimmermann and Uhlenbeck (1998) HhRz model with outcomes for the WT(GAAA) and A7U (GAAA/AGUA) hRHO 266 EhhRzs for both the 15-mer and 14-mer substrates. The kinetic model diagram (box) shows the Kd values and rate constants calculated for the A7U(GAAA/AGUA) EhhRzs solely for the 15-mer substrate. The A7U(GAAA) and A7U(AGUA) EhhRzs have identical antisense flanks and so have the same calculated kinetic properties. Kd values have units of nM. Rate constants (k) have units of min⁻¹.

FIG. 13 shows the chemical Modifications in the Catalytic Core and 5′ and 3′ ends of the EhhRz. Synthetic modified EhhRzs have 2′ O-Methyl in the AS flanks and AGU of the Stem-II cap (normal AGUA, A left unmodified). Chemical modifications were made within the core of the EhhRz (266 EhhRz “A7U AGUA”, control, 254.52±36.45 min⁻¹) and the catalytic turnover activity was assessed against the standard 15-mer substrate under standard conditions (10 mM Tris-HCl, pH 7.5, 0.5 mM MgCl₂, 37° C. The rate data was scaled by the measured fluorescence per mole of the upstream product fragment containing the FAM moiety. One Way ANOVA strongly refuted null hypothesis (p<<0.05). Relative to the “WT” EhhRz A7U AGUA variant made with natural RNA nucleotides most synthetic construct modifications showed statistically significant difference (Bonferroni, p<0.05, Asterisks). Some modifications severely attenuated turnover rate (e.g., mU4, 2′-MOE U4, mA6, mU4,7). Some modifications had no significant impact (e.g., 2-OMe changes throughout both antisense sequences and the first three nucleotides of the AGU tetraloop). Several chemical modifications enhanced cleavage turnover rate (e.g., mU7, mA13, 3′ Inverted dT, LNA 5′ and 3′).

FIG. 14 shows the impact of chemical modification at the U4 residue within the enzyme core of the EhhRz. Multiple chemical modifications were made at U4 within the core of the EhhRz (266 EhhRz A7U AGUA) and the impact assessed on turnover rate against the 15-mer substrate (0.5 mM Mg²). One Way ANOVA strongly refuted null hypothesis (p<<0.05). Relative to the “WT” EhhRz A7U AGUA variant made with natural RNA nucleotides the synthetic construct modifications showed statistically significant differences (Bonferroni, p<0.05, Asterisks) except the modification with a single PS (phosphorothioate) bond 3′ to the U4 base. Pseudouridine base has over 50% of control activity. Modifications of the 2′ position on the ribose of U4 are uniformly deleterious to rate. L-RNA refers to mirror image Levo-RNA at position U4. An idSpacer in the environment of U4 is deleterious. These findings point to important roles of the 2′OH on the U4 ribose and the structural environment around U4 in reaction chemistry; replacing uridine with pseudouridine has less effect and the charge distribution of the phosphodiester vs phosphorothioate link with G5 has no impact. Mean rates (rounded to nearest whole digit) were as follows: Unmodified EhhRz (250 min⁻¹), 2′ O-Methyl (17 min⁻¹), 2′-O-methoxy-ethyl (2′-MOE) (13 min⁻¹), 2′-Fluoro U (63 min⁻¹), 2′-Amino U (61 min⁻¹), Thymidine (14 min⁻¹), L-RNA (33 min⁻¹), PS Bond (262 min⁻¹), PseudoUridine (154 min⁻¹), and idSpacer (21 min⁻¹).

FIG. 15 shows that chemical modifications secure nuclease resistance of 266 A7U AGUA. Evolution of the synthetic EhhRzs catalytic activity with incorporated resistant chemistries. The Syn-EhhRz's in the figure have 2′ O-Methyl in the AS flanks and AGU of the cap except for the unmodified EhhRz (ribose throughout with rate at 257 min-1) and Stem DNA at C (ribose everywhere but 4 C's in stem at 353 min-1). A7U AGUA with 2′-OMe in antisense flanks and the AGU of the tetraloop had 284 min-1. 2′-Fluoro U4 had 63 min-1. 2′OMe at U7 had 301 min-1. Locked nucleic acid (LNA) at 5′ and 3′ termini had 323 min-1. 3′-Inverted dT had 352 min-1. The final composition of a nuclease resistant Syn-EhhRz #1 (155 min-1) consists of the following modifications. LNA at both 5 and 3 prime ends, 2′ 0-Methyl in the AS flanks and AGU of the cap, DNA at all Cs in Stem-II, 2′-Fluoro at U4 and 2′ O-Methyl at U7. The final composition of nuclease resistant Syn-EhhRz #2 (158 min⁻¹) consists of the following modifications. LNA at the 5′ end, Inverted dT at the 3′ end, 2′ 0-Methyl in the AS flanks and AGU of the cap, DNA at all Cs in Stem-II, 2′-Fluoro at U4 and 2′ O-Methyl at U7. Asterisks show significant differences (p<0.05) compared to A7U AGUA.

FIGS. 16A-B show divergence of cleavage turnover activity with resistance chemistries for Syn-EhhRzs. FIG. 16A shows the catalytic activity of two nuclease resistant Syn-EhhRzs that have markedly different turnover rates. Cleavage activity of a nuclease resistant (#1) Syn-EhhRz (155 min⁻¹) vs a 2′ O-Methyl nuclease resistant enzyme with no catalytic activity (4 min-1). FIG. 16B shows that active Syn-EhhRz remains intact in 10% Normal Human Serum (NHS) for 24 hrs at 37° C. (Lane 8) vs. an inactive 2′-O-Methyl Syn-EhhRz (Lane 6), which remains intact, versus an unmodified Syn-EhhRz (Lane 4), which is completely degraded.

FIG. 17 shows trans Cleavage of 15 nt Substrate of 266 EhhRzs [A7U(AGUA)] after self-Cleavage from mCherry-266 Lollypop-EhhRz fusion Reporter-Target-EhhRz mRNA. An in vitro transcript of the fusion-reporter mRNA was made using PCR constructs generated with a T7 RNA polymerase promoter. In vitro cleavage of the fusion reporter mRNA was proven by PAGE gel based analysis. The cleaved RNA preparation was used in a standard moderate throughput ribozyme kinetic turnover assay. 15 mer substrate with 5′ FAM and 3′ BHQ1 was used for both the 266 EhhRzs and this substrate has no intrinsic structure. Substrate was in 10:1 fold molar excess over hhRz (1 μM: 100 nM) and Mg²⁺ was 0.5 mM and reaction temperature was 37° C. Rates plotted are the measured initial rates of the reaction. The EhhRz 266 agents had raw turnover rates scaled to (divided by) product fluorescence (3.11 FluorUnits/nM). The turnover rate with 1× EhhRz, 2× EhhRzs, and 4× EhhRzs embedded in the 3′UTR of the construct are shown. There is a proportional increase in in trans cleavage rate as the number of EhhRzs embedded within the cis construct increases.

FIG. 18 shows in cellulo evaluation of EhhRz function at the protein level. The mCherry-266 stem loop structure—EhhRz fusion Reporter-Target-plasmids were transfected into naïve HEK293S cells. CMV expression plasmid constructs contained 1, 2 or 4 active 266 EhhRzs [A7U(AGUA)] or 1, 2, or 4 Inactive 266 EhhRzs [A7U(AGUA) (G8C inactivating mutations). After 48 hours plates were imaged for cellular mCherry fluorescence using appropriate optical excitation/emission filters. The data shows progressive reduction of mCherry fluorescence (indirect assay for mCherry protein) with increasing numbers of embedded active EhhRzs targeting 266 CUC site in hRHO. No change was observed in the levels of expression with the inactive EhhRzs embedded, showing that antisense effects are not a dominant mechanism of suppression.

FIG. 19 shows in cellulo evaluation of EhhRz function at the mRNA level. The mCherry-266 stem loop structure—EhhRz fusion reporter-target-plasmids were transfected into naïve HEK293S cells. CMV expression plasmid constructs contained 1, 2 or 4 Active 266 EhhRzs [A7U(AGUA)] or 1, 2, or 4 Inactive 266 EhhRzs [A7U(AGUA) (G8C inactivating mutations). After 48 hours total RNA was isolated from transfected cells and RT/PCR was performed relative to a β-actin housekeeping standard. The results show a progressive increase of the mCherry mRNA Ct levels with increasing numbers of embedded active EhhRzs targeting 266 CUC site in hRHO This data demonstrates a progressive knockdown of the mCherry mRNA with increasing numbers of self-cleaving EhhRzs. No change in the levels of mCherry expression with the inactive EhhRzs embedded (G8C) was observed. Fold change (2(−ΔΔCt) method showed a relative fold level of 1 for the inactive control, 69% mCherry knockdown for the 1× EhhRz construct, 90% knockdown for the 2× EhhRz construct, and 98% knockdown for the 4× EhhRz construct.

FIG. 20 shows the structure-function effects of various stem II compositions with an AGUA tetraloop. Various Stem II compositions were investigated for effects on turnover rate in vitro with the 15 nt pre-fluorescent substrate. The A7U(AGUA) 266 EhhRz is considered the WT enzyme in the Examples and for purposes and comparisons described herein. The WT stem II was 5′-GGCC-AGUA-GGCC-3′ (SEQ ID NO: 17) with the composition of the two sides of the stem (hybridized) and the AGUA tetraloop (single stranded) residing between. Stem II, 5′-GUCC-AGUA-GGAC-3′ (SEQ ID NO: 18), promoted a statistically significance increase in turnover rate compared to the WT 266 EhhRz. The 5 bp stem II 5′-GGCCC-AGUA-GGGCC-3′ (SEQ ID NO: 19) led to significant decrease in turnover rate. The 3 bp Stem II 5′-GGC-AGUA-GCC-3′ (SEQ ID NO: 20) had a marked decrease in activity.

FIG. 21 shows a comparison of 266 hRHO EhhRzs to Gorbatyuk et al. (2007) 525 mhhRz. A 15-nt substrate with 5′ FAM and 3′ BHQ1 was used for the 266 EhhRzs and a 13-nt substrate (with 5′ FAM and 3′ BHQ) was used for the 525 mhhRz. Substrate was in 10:1 fold molar excess over hhRz (1 uM: 100 nM) and Mg²⁺ was 0.5 mM and reaction temperature was 37° C. Rates plotted are the measured initial rates of the reaction. The EhhRz 266 agents had raw turnover rates scaled to (divided by) product fluorescence (3.11 FluorUnits/nM) and the 525 mhhRz data was scaled also to its product fluorescence (0.77 FluorUnits/nM). Three EhhRzs show improved turnover rate relative to the 525 mhhRz (* means statistically significant with p values stated). The upstream regions and tetraloop composition for the 266 EhhRzs against hRHO are shown. The 525 hhRz used a UUCG tetraloop which severely depressed activity of EhhRz 266. The enzyme core was identical between the EhhRz 266 and 525 mhhRzs and the stem II region had comparable G:C content (stability). The antisense flanks differed due to the differing targeting sequences.

FIGS. 22A-E show 3D RNA folding of human PRPH2 mRNA Target. FIG. 22A shows the overall multiparameter prediction of RNA accessibility (mppRNA) map with colored arrows and bar showing regions of interest. The red bar shows the mean accessibility across the entire mRNA; the mean±SEM is shown. FIG. 22B show the high accessibility region around the red arrow in the overall map; there is a strong region of accessibility between 75-125 nt which is within the 5′UTR. FIG. 22C shows the high accessibility region around the blue arrow in the overall map; there is a strong accessible region between 950-1025, which is in the coding region. FIG. 22D shows the broad region between 1325-1425 nt (green arrow), which is 3′UTR region. FIG. 22E shows an unusual symmetrical peak structures between 1700-1950 (purple bar in (FIG. 22A)); U11 indicates a loop with 11 U residues, which is also 3′UTR.

FIG. 23 shows Selected NUH cleavage sites for an EhhRz screen in human PRPH2 mRNA target. The 5′ UTR and 3′ UTR accessibility regions were chosen for EhhRz screening. There are four NUH cleavage sites in the 5′UTR accessibility region and two of these have a U7 residue downstream of the cleavage site, and neither of these have secondary structures. These two sites were chosen for EhhRz screen. There were are ten NUH cleavage sites in the 3′UTR accessibility region and four of these have a U7 residue downstream of the cleavage site, and three of these have no secondary structure and one has 2 bp. A total of 6 EhhRzs were screened for hPRPH2. 5′-CCCUGCUCAAGCUGUGAUUCCGAGACCCCUGCCACCACUACUGCAUUCACGGGG AU-3′ (SEQ ID NO: 21), CCCUGCUCAAGCUGU (SEQ ID NO: 22), 5′-CACCACUACUGCAUU-3′ (SEQ ID NO: 23), 5′-CCGAACACUGAGAAAUAGUGCACUCCAAGAAACGUGGAUCUCCCCCUCAUCCAA CUCCGAAAGUCUGAAUCUCCCA-3′ (SEQ ID NO: 24), GAGAAAUAGUGCACU (SEQ ID NO: 25), CGUGGAUCUCCCCCU (SEQ ID NO: 72), UCCAACUCCGAAAGU (SEQ ID NO: 26), and CGAAAGUCUGAAUCU (SEQ ID NO: 27) are shown. Underlining indicates NUH cleavage site.

FIG. 24 shows a NUH cleavage site screen for EhhRz against Human PRPH2 mRNA target. The two candidate EhhRzs designed against the CUA↓ and CUC↓sites in the 5′ UTR (shown in FIG. 23) and the four candidate EhhRzs designed against the GUC↓, CUC↓, AUC↓, and AUA↓ were tested for sites in the 3′ UTR (shown in FIG. 23) of PRPH2 mRNA. 15 mer substrates were synthesized for each of the six target sites with 5′ FAM and 3′ BHQ1. Mean cleavage rates were measured optically and compared to the scaled rate of our best 266 CUC↓ A7U(AGUA) EhhRz for hRHO. The PRPH2 hhRzs were made with the AGUA tetraloop. Asterisks indicate statistical significance (p<0.05) relative to the hRHO EhhRz. Ampersand (&) indicates statistical significance relative to the 5′UTR CUA↓ EhhRz for PRPH2.

FIGS. 25A-B show Michaelis-Menten analysis of hPRPH2 5′UTR CUA↓ A7U(AGUA) EhhRz. FIG. 25A shows the cleavage rate of lead hPRPH2 EhhRz vs 15mer substrate concentration. The Michaelis-Menten hyperbolic function was well fit to the data set allowing extraction of the Vmax (P1) and Km (P2) parameters. The enzyme efficiency (Vmax/Km) was 1.38×10⁸ M⁻¹min⁻¹. FIG. 25B shows a comparison of the Michaelis-Menten analysis 266 hRHO A7U(AGUA) EhhRz with data scaled to product fluorescence. Note the different scales. The enzyme efficiency is 1.89×10⁸ M⁻¹min⁻¹. These enzyme efficiencies are both on the scale of the protein RNaseA (1.4×10⁸ M⁻¹min⁻¹).

FIGS. 26A-D show the initial EhhRzs designed against human TCF4 mRNA. EhhRzs were designed against a CUC cleavage target site immediately downstream of the intronic expansion element that occurs in Fuchs Endothelial Corneal Dystrophy. FIG. 26A shows multiparameter prediction of mRNA accessibility (mppRNA) demonstrating a toxic CUG expansion (100 elements, RED bar) in the TCF4 intron 2. There is a CUC↓ EhhRz cleavage site immediately downstream (small violet bar). FIG. 26B shows testing of TCF EhhRzs against a CUC site using the identical formulation used for hRHO with 3G residues upstream of the antisense flank with both WT annealing and A7U annealing, both with AGUA tetraloops. The results show excellent turnover rate (obtained prior to scaling data). FIG. 26C shows a comparison of different EhhRzs against TCF4 CUC↓ with WT annealing or A7U annealing and with AGUA tetraloop but with either 1 or 3 Gs at the 5′ end (here scaled data). The number of Gs affects turnover rate (1G better rate than 3G with this target site) and the A7U is better in both cases that the WT annealing pattern. Data was scaled for product fluorescence in FIG. 26C and FIG. 26D. FIG. 26D shows a comparison of the evolution of turnover rate (scaled data) with 1G residue at 5′ end, with WT or A7U annealing, and with GAAA and AGUG tetraloops. This progression of activity is similar to the findings of hRhHO 266 EhhRz evolution.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

The terms “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides, ribonucleotides or a combination thereof, and may be produced enzymatically or synthetically (e.g., PNA as described in U.S. Pat. No. 5,948,902 and the references cited therein) and which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U, respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as an inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acids are described in US20050233340, which is incorporated by reference herein for disclosure of UNA.

As used herein, the term “oligonucleotide” refers to a multimer of at least 10, e.g., at least 15 or at least 30 nucleotides. In some aspects, an oligonucleotide may be in the range of 15-200 nucleotides in length, or more.

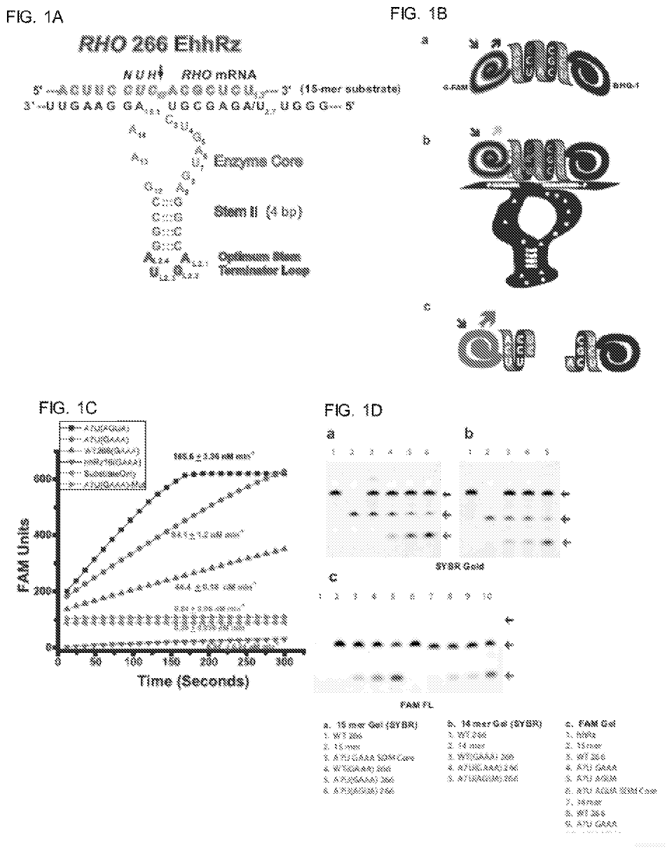

As used herein, the term “hammerhead ribozyme” refers to a RNA molecule motif that catalyzes reversible cleavage and joining reactions at a specific site within an RNA molecule. For example, hammerhead ribozymes are catalytic RNA molecules capable of inducing site-specific cleavage of a phosphodiester bond within an RNA molecule. The disclosed hammerhead ribozymes can be used to reduce intracellular level of a specific mRNA coding for a protein. The minimal hammerhead ribozyme is composed of three base paired helices, separated by short linkers of conserved sequence as shown in the crystal structure described in Scott (Cell 1995 81: 991-1002). These helices are called I, II and III. The conserved uridine-turn links helix I to helix II and usually contains the sequence CUGA. Helix II and III are linked by a sequence GAAA. The cleavage reaction occurs between helix III and I, and is usually a C. The structure-function relationships in ribozymes have been extensively reviewed (see, e.g., Hammann et al, RNA 2012 18: 871-885). The structure of an exemplary minimal hammerhead ribozyme is shown in FIG. 1. The various parts of a hammerhead ribozyme, e.g., enzyme core, stem II, stem terminator loop, etc. are defined with reference to FIG. 1. A hammerhead ribozyme can also contain one or more non-naturally occurring nucleotides, as described herein.

As used herein, the term “patient” refers to a subject afflicted with a disease or disorder. The term “patient” includes human and veterinary subjects. In some aspects of the disclosed methods, the “patient” has been diagnosed with a need for treatment for a disease caused by increased intracellular levels of a protein that is associated with or causing a disease or disorder, such as, for example, prior to the administering step.

“Treatment” and “treating” refer to administration or application of a therapeutic agent (e.g., a nucleotide or polynucleotide or hammerhead ribozyme described herein) to a subject or performance of a procedure or modality on a subject for the purpose of obtaining a therapeutic benefit of a disease or health-related condition. For example, a treatment may include administration of a pharmaceutically or therapeutically effective amount of a nucleotide or polynucleotide or hammerhead ribozyme that is capable of binding to a target mRNA sequence comprising a NUH↓ cleavage site.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a disease by increased intracellular levels of a protein that is associated with or causing a disease or disorder.

The terms “preventing,” “blocking,” “antagonizing,” or “reversing” mean preventing in whole or in part, or ameliorating or controlling.

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Mutants,” “derivatives,” and “variants” of a nucleotide are nucleic acids which may be modified or altered in one or more nucleotides such that the nucleic acid is not identical to the wild-type sequence, but has homology to the wild type nucleic acid.

The term “variant” can refer to a nucleotide that displays modifications in sequence and/or functional properties (i.e., altered characteristics) when compared to the wild-type nucleotide. In general, it is understood that one way to define any known variants and derivatives or those that might arise, of the disclosed nucleotides herein, is through defining the variants and derivatives in terms of homology to specific known sequences. This identity of particular sequences disclosed herein is also discussed elsewhere herein. In general, variants of nucleotides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two nucleic acids. For example, the homology can be calculated after aligning the two sequences so that the homology is at its highest level. In some aspects, the term “variant” can mean a difference in some way from the reference sequence other than just a simple deletion of an N- and/or C-terminal residue(s). In an aspect, a variant can include a substitution of a nucleotide residue. Variants can include at least one substitution and/or at least one addition, there may also be at least one deletion. Variants can also include one or more non-naturally occurring residues.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more, and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

As used herein, the term “host cell” refers to any cell that harbors, or is capable of harboring, one or more of the hammerhead ribozymes disclosed herein. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV minigene plasmid, an accessory function vector, or other transfer DNA or RNA associated with the production of recombinant AAVs. The term includes the progeny of the original cell which has been transfected. Thus, a “host cell” as used herein can refer to a cell which has been transfected with an exogenous DNA or RNA sequence. It is understood that the progeny of a single parental cell may not necessarily be completely identical in morphology or in genomic or total DNA complement as the original parent, due to natural, accidental, or deliberate mutation.

Described herein are enhanced hammerhead ribozyme (EhhRz) kinetic performance during therapeutic optimization of a lead agent against the human rod opsin (RHO) mRNA target. Turnover activity in trans was greater than 300 nMmin⁻¹ under 10-fold substrate excess and physiological Mg²⁺ (1 mM). A moderate-throughput fluorescence quantitative hhRz kinetic assay was validated, which is linear with substrate and product moles. The EhhRz targets a CUC cleavage site in a substrate with no predicted secondary/tertiary structure and demonstrates classical Michaelis-Menten turnover behavior with efficiency (Vₘₐₓ/Kₘ) up to 1.60×10⁸ min⁻¹M⁻¹, which is comparable to RNase A. EhhRzs show cooperative Mg²⁺ titration (K_{d}=0.73±0.02 mM; Hill=1.73±0.07). Structure-function assays showed that the upstream EhhRz antisense flank (substrate bound) interacts with stem-loop II. Loop variation reveals a marked effect on turnover rate, which must relate to base interaction chemistries as structural accessibility is uniform. Downstream substrate U7 is not important for enhanced activity. Under single-turnover conditions reaction rates exceeded 1,000 min⁻¹. Embedded within a target-reporter fusion mRNA EhhRzs cleave structured target elements under intracellular conditions promoting strong knockdown at target mRNA and protein levels. EhhRzs can be used as druggable nucleic acid therapeutics against arbitrary targets, or in the design of improved aptazymes.

Small catalytic RNAs (class of noncoding RNAs) have been studied for more than 3 decades since the paradigm-shifting discoveries of Altman and Cech that RNA chemistry was capable of catalysis. The hammerhead ribozyme (hhRz) is a catalytic RNA with a highly conserved 11-nt enzyme core sequence (Uhlenbeck, 1987). The minimal hhRz (mhhRz) was isolated from larger virusoid RNAs that function efficiently but variably in rolling circle RNA replication in cis. The mhhRz can function as a Michaelis-Menten enzyme under substrate-excess conditions with a minimal target RNA with no structure and non-physiological free Mg^{2+ levels (generally >}10 mM). It was thought that the mhhRz could serve as an engineered site-specific RNA endonuclease for nucleic acid therapeutics. However, mhhRzs exhibit slow catalysis, generally a 1-2min⁻¹ turnover for short targets pre-annealed with excess enzyme and often orders of magnitude lower for structured RNAs with constrained accessibility to annealing. The latter can be at least partially mitigated by accessibility prediction algorithms and experimental target mRNA mapping approaches. Because any therapeutic catalytic RNA will add a component to the intrinsic rate of degradation, stable mRNAs (long half-lives) encoding structural or enzymatic proteins were thought to be viable targets. The extremely slow turnover rate at physiological intracellular Mg²⁺ levels (˜1 mM) is a major factor in therapeutic applications of mhhRzs because it necessitates high therapeutic enzyme [E] levels to achieve significant knockdown of mRNA substrate [S], even at rare, fully accessible regions. These are conditions far from Michaelis-Menten kinetics (i.e., [S]>>[E]). The need for increased delivery or expression of the therapeutic results in greater technological challenges but also increased risks for off-target effects and vector- or therapeutic-related toxicity.

The discovery of upstream tertiary accessory elements (TAEs) of native or extended hhRzs (xhhRzs), long missing from 15 years of study of the mhhRz, has proven important to understanding RNA catalytic function at cellular levels of Mg²⁺ (0.4-2 mM). xhhRzs tend to have improved catalytic rates when operating natively in cis (intramolecular), but adapting to the therapeutic in trans (intermolecular) configuration has limitations. The upstream antisense flank must be extended to insert the TAE, which could decrease specificity and suppress product leaving rates and thereby constrain enzyme turnover capacity under steady-state conditions. The structure of xhhRzs revealed that the upstream TAE interacts with the loop capping stem-loop II of the enzyme core to stabilize a catalytically active conformation at low Mg²⁺, which was populated in the mhhRz at high Mg²⁺. Studies have since illustrated that there are important residues in the highly conserved cores of the xhhRz and mhhRz, such as G12 and G8, which function as general base and acid drivers, respectively, for RNA phosphodiester cleavage, similarly to the roles of histidine residues in the enzyme core of RNase A.

Because many naturally occurring xhhRzs have a U7 residue in the target substrate sequence 7 nt downstream of the cleavage site it has been thought that an unpaired substrate U7 residue forms a Hoogsteen face base interaction with the fourth A in the GUGA (or GUUA) tetraloop capping stem II loop. Single-turnover rates (enzyme excess under pre-annealed conditions) at 10 mM Mg²⁺ and 27° C. were 61-95 min⁻¹, with rates extrapolated mathematically from gel-based measurements at pH 5.6. It has been demonstrated that rate enhancements of hhRzs do not require complex structured upstream TAEs, but they do require certain in trans tertiary interactions.

Described herein are enhanced hhRzs (EhhRzs) in trans, without upstream TAEs, that can achieve rates>300 nM min⁻¹ under Michaelis-Menten ([S]>[E]) conditions at physiological temperature (37° C.) and Mg²⁺ concentrations (<1 mM) and pH. EhhRzs have catalytic turnover efficiency (Vₘₐₓ/Kₘ) on the same scale as the highly efficient protein enzyme RNase A. Moreover, that the results show that the substrate U7 residue is not important to log-order enhancement of catalytic turnover even under physiological (therapeutic) conditions. The tetraloop capping stem II loop substantially modulates activity regardless of the presence of a U7 residue. Optimum tetraloops are of a different class than those used previously in mhhRzs or found in xhhRzs. These findings demonstrate that the enzymatic capacity and structural dynamics of the hhRz are far from fully understood. The catalytic enhancements of EhhRzs can be used as human nucleic acid therapeutics.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### Compositions

Disclosed herein are hammerhead ribozymes that have an increased turnover rate of a substrate compared to a wild-type hammerhead ribozyme. In some aspects, the substrate can be a mRNA target sequence. In some aspects, the hammerhead ribozymes comprise a stem II region and a stem terminator loop. In some aspects, the hammerhead ribozymes further comprise an enzyme core. In some aspects, the hammerhead ribozymes can further comprise a 5′ flanking region. In some aspects, the hammerhead ribozymes can further comprise a 3′ flanking region. In some aspects, the hammerhead ribozymes bind to an mRNA target sequence. FIG. 1A shows a representative example of the structure of the disclosed hammerhead ribozymes. The hammerhead ribozyme sequences disclosed herein can be compared to a wild type sequence. In some aspect, the wild type sequence can be 5′-GGGUAGAGCGUCUGAUGAGGCCGAAAGGCCGAAAGGAAGUU-3′ (SEQ ID NO: 28).

Disclosed herein are hammerhead ribozymes comprising an enzyme core, a stem II region, a stem terminator loop, a first flanking region at the 5′ end and a second flanking region at the 3′ end, wherein the first flanking region comprises a uracil at position 7.

Disclosed herein are hammerhead ribozymes comprising a nucleic acid sequence of CUGAUGA GGCC X₁X₂X₃X₄ GGCC GAAA (SEQ ID NO: 29). In some aspects, X₁X₂X₃X₄ of SEQ ID NO: 29 can be AGUA (SEQ ID NO: 30), UAAA (SEQ ID NO: 31), AAAA (SEQ ID NO: 32), GUGA (SEQ ID NO: 33), GAGA (SEQ ID NO: 34), GUUA (SEQ ID NO: 35), GAUA (SEQ ID NO: 36), GACA (SEQ ID NO: 37), AAAG (SEQ ID NO: 38), AACA (SEQ ID NO: 39), AUUA (SEQ ID NO: 40), AAUA (SEQ ID NO: 41), or AGCA (SEQ ID NO: 42). In some aspects, the nucleic acid sequence has an enhanced turnover rate compared to a wild-type hammerhead ribozyme.

Stem II region. In some aspects, disclosed herein are hammerhead ribozymes comprising a stem II region, and a stem terminator loop, wherein the stem II region of the hammerhead ribozymes comprise 4 base pairs. The four base pairs of the stem II region are hybridized to their complement in the stem II region. For example, disclosed herein are hammerhead ribozymes comprising a stem II region, and a stem terminator loop, wherein the stem II region comprises 5′-GGCC-AGUA-GGCC-3′ (SEQ ID NO: 17) with the composition of the two sides of the stem II region (hybridized) and the AGUA tetraloop (single stranded) residing between the two sides of the stem II region. In some aspects, the GC base pair on the top of the stem II region can alter the activity of the disclosed hammerhead ribozymes. For example, a stem loop comprising GUCC can increase activity of the disclosed hammerhead ribozymes.

Stem terminator loop. In some aspects, disclosed herein are hammerhead ribozymes comprising a stem II region, and a stem terminator loop, wherein the hammerhead ribozymes comprise a stem terminator loop. As used here, the terms “stem terminator loop”, “stem II terminator loop”, “stem II tetraloop”, “stem tetraloop”, “tetraloop”, and “cap” can be used interchangeably. In some aspects, the stem terminator loop connects the four base pairs of the stem II region. In some aspects, the stem terminator loop comprises or consists of AGUA, UAAA, AAAA, GUGA, GAGA, GAUA, GUUA, GACA, AAAG, AACA, AUUA, AAUA, or AGCA. In some aspects, the stem terminator loop comprises or consists of AGUA, UAAA, AAAA, GAUA, GUUA, AUUA, AAUA, or AGCA. In some aspects, the stem terminator loop does not comprise UUCG or CUUG.

Enzyme core. In some aspects, disclosed herein are hammerhead ribozymes comprising a stem II region, and a stem terminator loop, wherein the hammerhead ribozymes further comprise an enzyme core. In some aspects, the enzyme core comprises the sequence 5′-CUGAUGA-3′ corresponding to nucleic acids C3 to A9, and 5′-GAA-3′ corresponding to nucleic acids G12 to A15.1 as shown in FIG. 1A. For example, disclosed are hammerhead ribozymes wherein the hammerhead ribozyme comprises the nucleotide sequence of 5′-CUGAUGAGGCC X₁X₂X₃X₄GGCCGAAA (SEQ ID NO: 29), wherein the underline nucleotides correspond to the enzyme core, bold nucleotides correspond to the stem II region, and “X₁X₂X₃X₄” nucleotides correspond to the stem tetraloop. In some aspects, X₁ can be any nucleotide. In some aspects, X₂ can be any nucleotide. In some aspects, X₃ can be any nucleotide. In some aspects, X₃ can be a U (SEQ ID NO: 43). In some aspects, X₄ can be any nucleotide. In some aspects, X₄ can be an A (SEQ ID NO: 44.

Flanking regions. In some aspects, disclosed herein are hammerhead ribozymes comprising a stem II region, and a stem terminator loop, wherein the hammerhead ribozymes further comprise a first flanking region and a second flanking region. The first flanking region and second flanking region are located on the end of the enzyme core. Specifically, the first flanking region is 5′ of the enzyme core and the second flanking region is 3′ of the enzyme core as depicted in FIG. TA. In some aspects, a first flanking region can be located on the 5′ end of the hammerhead ribozyme. In some aspects, a second flanking region can be located on the 3′ end of the hammerhead ribozyme. In some aspects, the first and second flanking regions comprise a first and a second complementary nucleotide sequence to a target mRNA sequence, respectively.

Conventional hammerhead ribozymes cleave after NUH cleavage sites, where N is any nucleotide and H is any nucleotide except guanosine. As described herein, the first and second flanking regions are antisense flanking regions that allow the hammerhead ribozymes disclosed herein to anneal by Watson Crick base pairing to the accessible region of the target mRNA which contains an NUH↓ cleavage site. For example, the first and second flanking regions can be designed to hybridize and be complimentary to a target mRNA sequence, wherein the target mRNA sequence comprises a NUH↓ cleavage site. Thus, in some aspects, the first flanking region and the second flanking region can comprise a first and a second complementary nucleotide sequence to a target mRNA sequence, wherein the target mRNA sequence comprises a NUH cleavage site.

In some aspects, the first and second flanking regions comprise mRNA sequences that are capable of hybridizing to a mRNA target sequence. In some aspects, target mRNA sequences include but are not limited to rhodopsin, peripherin-2, centrosomal, or transcription factor 4. In some aspects, the first flanking region comprises from 5′ to 3′ the nucleic acid sequence of GGGUUGAGCGU (SEQ ID NO: 45) (e.g., hRHO). In some aspects, the second flanking region comprises from 5′ to 3′ the nucleic acid sequence AGGAAGUU (e.g., hRHO). In some aspects, the first flanking region comprises from 5′ to 3′ the nucleic acid sequence of GGGUGAAGUG (SEQ ID NO: 46) (e.g., hRHO; 725 GUC↓). In some aspects, the second flanking region comprises from 5′ to 3′ the nucleic acid sequence ACCACGA (e.g., hRHO; 725 GUC↓). In some aspects, the first flanking region comprises from 5′ to 3′ the nucleic acid sequence of GGGUACGUGG (SEQ ID NO: 47) (e.g., hRHO; 1362 GUC↓). In some aspects, the second flanking region comprises from 5′ to 3′ the nucleic acid sequence ACUCCAG (e.g., hRHO; 1362 GUC↓). In some aspects, the first flanking region comprises from 5′ to 3′ the nucleic acid sequence of GGGUUAUGCAG (SEQ ID NO: 48) (e.g., PRPH2; CUA 5′UTR antisense flanks). In some aspects, the second flanking region comprises from 5′ to 3′ the nucleic acid sequence AGUGGUG (e.g., PRPH2; CUA 5′UTR antisense flanks). In some aspects, the first flanking region comprises from 5′ to 3′ the nucleic acid sequence of GUUGAGGAG (e.g., TCF4; CUC site). In some aspects, the second flanking region comprises from 5′ to 3′ the nucleic acid sequence AGGAGGA (e.g., TCF4; CUC site).

Disclosed herein are hammerhead ribozymes comprising an enzyme core, a stem II region, a stem terminator loop, a first flanking region at the 5′ end and a second flanking region at the 3′ end, wherein the first flanking region comprises a uracil at position 7. The numbering system for the disclosed hammerhead ribozymes can be the numbering system described in Hertel K J, et al., Nucleic Acids Res. 1992; 20(12): 3252, which is hereby incorporated by reference for its teaching of hammerhead ribozymes. For example, the first flanking region can comprise the nucleotide sequence of 5′-GGGUUGAGCGU-3′ (SEQ ID NO: 45), as shown in FIG. 1A, wherein an “A” at position 7 is replaced with a “U”. As used herein, such a substitution is referred to as an “A7Usubstitution”.

In some aspects, the hammerhead ribozyme comprises the nucleic acid sequence of

(SEQ ID NO: 3)
5′-GGGUUGAGCGUCUGAUGAGGCCX₁X₂X₃X₄GGCCGAAAGGAAGUU.

In some aspects, X₁X₂X₃X₄ of SEQ ID NO: 3 are AGUA (SEQ ID NO: 4), UAAA SEQ ID NO: 5), AAAA SEQ ID NO: 6), GUGA SEQ ID NO: 7), GAGA SEQ ID NO: 8), GUUA SEQ ID NO: 9), GAUA SEQ ID NO: 10), GACA SEQ ID NO: 11), AAAG SEQ ID NO: 12), AACA SEQ ID NO: 13), AUUA SEQ ID NO: 14), AAUA SEQ ID NO: 15), or AGCA SEQ ID NO: 16). In some aspects, the nucleic acid sequence has an enhanced turnover rate compared to a wild-type hammerhead ribozyme. From 5′ to 3′, the first flanking region of SEQ ID NO: 3 is shown in bold and underline; the enzyme core of SEQ ID NO: 3 is shown in bold; stem II region of SEQ ID NO: 3 shown with underline; the stem terminator loop of SEQ ID NO: 3 is indicated as “X₁X₂X₃X₄”; and the second flanking region of SEQ ID NO: 3 is shown in bold and underline. See, also, FIG. 1A.

Target mRNA sequences. As used herein, the phrase “target mRNA sequence” refers to an mRNA sequence to which a hammerhead ribozyme as disclosed herein can bind and comprises a NUH↓ cleavage site. The target mRNA sequences disclosed herein comprise a hammerhead ribozyme cleavage site. In some aspects, the ribozyme cleave site comprises a NUH↓ cleavage site, wherein N is any nucleotide, U is U, and H is C, A or U (not G). In some aspects, the target mRNA sequence encodes any mRNA sequence that comprises a NUH↓ cleavage site. Examples of target mRNA sequences include but are not limited to rhodopsin, peripherin-2, centrosomal, or transcription factor 4. In some aspects, NUH cleavage sites can occur every 12 nucleotides (e.g., every 8 to 16 nucleotides). In some aspects, mRNA can have a plurality of NUH cleave sites. For example, hRHO can have up to 236 NUH cleavage sites; many of which may be buried in structure and can be in accessible.

Modified nucleobases. In some aspects, the hammerhead ribozymes disclosed herein can be nuclease resistant. In some aspects, the nuclease resistance can be conveyed by modifying one or more nucleobases of the disclosed hammerhead ribozymes. In some aspects, the hammerhead ribozymes disclosed herein can comprise one or more modified nucleobases. In some aspects, the modified nucleobase can be any nucleotides (e.g., G, A, U, or C). In some aspects, the modified nucleobase can be a uracil residue or a cytosine residue. In some aspects, one or more regions of the disclosed hammerhead ribozymes can comprise a modified nucleobase. For example, the enzyme core, the stem terminator loop, and the first and second flanking regions can comprise one or more modified nucleobases. In some aspects, one or more modified nucleobases can be in the enzyme core and in one or more of the first or second flanking regions. In some aspects, certain nucleotides (e.g., U4, A6, A9, L4, G5, G8, G12 as shown in FIG. TA) in the enzyme core cannot be modified without impact on function (e.g., turnover rate). In some aspects, any of X₁X₂X₃ of the stem terminator loop can be modified. In some aspects, any of the nucleotides in the stem II region can be modified.

In some aspects, the one or more modified nucleobases can be a 2′-amino, a 2′-O-methyl, a 2′-O-methoxy-ethyl, a phosphorothioate bond, a 2′-Fluoro, a locked nucleic acid, an inverted dT, or a combination thereof. Examples of modified nucleobases included but are not limited to L-RNA (mirror image RNA, levo as opposed to dextro), 2,6-diamino-purines, 2-amino-purine, or synthetic nucleotides, N3′-P5′ phosphoroamidate uridine, 2′-O-allyl-uridine, and 2′-C-allyl-uridine.

In some aspects, locked nucleic acid chemistry can be applied to the 5′ and 3′ end sequences of the disclosed hammerhead ribozymes. For example, the hammerhead ribozymes disclosed herein can comprise an LNA nucleotide, wherein the LNA nucleotide is modified with an extra methylene bridge connecting the 2′ oxygen and 4′ carbon. In some aspects, the extra methylene bridge can fix the ribose moiety either in the C3′-endo (beta-D-LNA) or C2′-endo (alpha-L-LNA) conformation.

In some aspects, an inverted dT can be incorporated at the 3′end of any of the hammerhead ribozymes disclosed herein. The inverted dT provides a 3′-3′ linkage that inhibits degradation by 3′ exonucleases.

In some aspects, the first and/or second flanking regions can also comprise one or more modified nucleobases. For example, the first and/or second flanking regions can also comprise a 2′-amino, a 2′-O-methyl, a 2′-Fluoro, a phosphorothioate linkage, a 2′-O-methoxy-ethyl, a LNA, a L-RNA, or a combination thereof to the ribose bases.

In some aspects, the enzyme core can also comprise one or more modified nucleobases. For example, referring to FIG. TA, position C3 can be modified with 2′-OMe, position U4 can be modified with 2′-Fluoro, and position U7 can be modified with a 2-′OMe. In some aspects, position G12 can remain unmodified. In some aspects, positions A6 and A9 can remain unmodified. In some aspects, positions A13 and A14 can be modified with 2′-OMe and position A15 can remain unmodified.

In some aspects, the stem terminator loop can comprise one or more modified nucleobases. For example, in some aspects, X₁, X₂, X₃ can each be modified with 2′-OMe, and X₄ can remain unmodified. In some aspects, the hammerhead ribozymes disclosed herein can comprise modifications that include a deoxyribonucleic acid in place of a ribonucleic acid (e.g., the C RNA can be a C DNA).

In some aspects, the nucleotides disclosed herein can be further modified to improve stability. As used herein, the term “stability” refers to storage stability (e.g., room-temperature stability) as well as in vivo stability.

Disclosed herein are compositions comprising one or more of the hammerhead ribozymes described herein. In some aspects, the compositions can further comprise a pharmaceutically acceptable carrier. In some aspects, the pharmaceutically acceptable carrier can be lipid-based or a polymer-based colloid. Examples of colloids include liposomes, hydrogels, microparticles, nanoparticles and micelles. In some aspects, any of the hammerhead ribozymes can be encapsulated by a nanoparticle.

Vectors. Disclosed herein are vectors comprising any of the hammerhead ribozymes described herein. Vectors comprising nucleic acids or polynucleotides as described herein are also provided. As used herein, a “vector” refers to a carrier molecule into which another DNA or RNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polvadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In some aspects, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

Vectors for stable integration include but are not limited to plasmids, retroviruses, other animal viruses, etc.

The vectors disclosed herein can also include detectable label or selectable marker or label. A detectable marker or label can be introduced into the locus, where upregulation of expression can result in a detected change in the phenotype. Any of the vectors disclosed herein can also include a detectable marker or label. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus. The label can comprise any detectable moiety, including, for example, fluorescent labels, radioactive labels, and electronic labels.

Vectors include, for example, viral vectors (such as adenoviruses (“Ad”), adeno-associated viruses (AAV), and retroviruses, including lentiviruses), liposomes and other lipid-containing complexes, and other macromolecular complexes capable of mediating delivery of a polynucleotide to a host cell. Vectors can also comprise other components to further modulate the delivery and/or expression of the gene of interest, for example, or that otherwise provides beneficial properties to the targeted cells. A wide variety of vectors is known to those skilled in the art and is generally available. Other suitable complexes capable of mediating delivery of any of the nucleic acid constructs described herein include retroviruses (e.g., lentivirus), vaults, cell penetrating peptides and biolistic particle guns. Cell penetrating peptides are capable of transporting or translocating proteins across a plasma membrane; thus, cell penetrating peptides act as delivery vehicles. Examples include but are not limited to labels (e.g., GFP, MRI contrast agents, quantum dots).

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA or RNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA or RNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA or RNA such as a gene cassette containing a promoter, a DNA or RNA of interest and a polyadenylation signal. The rep and cap proteins may be provided in trans. Another significant feature of AAV is that it is an extremely stable and hearty virus. It easily withstands the conditions used to inactivate adenovirus (56.degree. C. to 65.degree. C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2: 619-623 (2000) and Chao et al., Mol Ther, 4: 217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector; as such a vector is contained within an AAV vector particle.

Recombinant AAV genomes of the invention comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1. AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle-specific expression, AAV1, AAV6, AAV8 or AAVrh.74 may be used.

DNA plasmids of the invention comprise rAAV genomes of the invention. The DNA plasmids are transferred to cells permissible for infection with a helper virus of AAV (e.g., adenovirus, El-deleted adenovirus or herpesvirus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

Methods of generating a packaging cell comprise creating a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79: 2077-2081), addition of synthetic linkers containing restriction endonuclease cleavage sites (Laughlin et al., 1983, Gene, 23: 65-73) or by direct, blunt-end ligation (Senapathy & Carter, 1984, J. Biol. Chem., 259: 4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158: 97-129). Various approaches are described in Ratschin et al., Mol. Cell. Biol. 4: 2072 (1984); Hermonat et al., Proc. Natl. Acad. Sci. USA, 81: 6466 (1984); Tratschin et al., Mol. Cell. Biol. 5: 3251 (1985); McLaughlin et al., J. Virol., 62: 1963 (1988); and Lebkowski et al., Mol. Cell. Biol., 7: 349 (1988). Samulski et al., J. Virol., 63: 3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13: 1244-1250 (1995); Paul et al. Human Gene Therapy 4: 609-615 (1993); Clark et al. Gene Therapy 3: 1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

Also disclosed herein are packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers and surfactants such as pluronics.

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1.times.10.sup.6, about 1.times.10.sup.7, about 1.times.10.sup.8, about 1.times.10.sup.9, about 1.times.10.sup.10, about 1.times.10.sup.11, about 1.times.10.sup.12, about 1.times.10.sup.13 to about 1.times.10.sup.14 or more DNase resistant particles (DRP) per ml. Dosages may also be expressed in units of viral genomes (vg).

Also disclosed herein are vectors comprising a single, cis-acting wild-type ITR. In some aspects, the ITR can be a 5′ ITR. In some aspects, the ITR can be a 3′ ITR. ITR sequences are about 145 bp in length. In some aspects, the entire sequences encoding the ITR(s) can be used in the molecule, although some degree of minor modification of these sequences is permissible. In some aspects, an ITR can be mutated at its terminal resolution site (TR), which inhibits replication at the vector terminus where the TR has been mutated and results in the formation of a self-complementary AAV. In some aspects, a “cis-acting” plasmid containing the transgene, in which the selected transgene sequence and associated regulatory elements can be flanked by the 5′ AAV ITR sequence and a 3′ hairpin-forming RNA sequence, can be used. AAV ITR sequences can be obtained from any known AAV, including presently identified mammalian AAV types. In some aspects, an ITR sequence can be an AAV2, AAV3, AAV4, AAV5, AAV6, AAV7, AAV8, AAVrh8, AAV9, AAV10, and/or AAVrh10 ITR sequence. In some aspects, the AAV ITR sequences can be AAV9.

Disclosed herein are pharmaceutical compositions comprising a therapeutically effective amount of any of the hammerhead ribozymes or vectors disclosed herein, and a pharmaceutically acceptable carrier and/or adjuvant.

In some aspects, the composition (e.g., a pharmaceutical composition) can comprise an AAV or lentiviral vector comprising any of the hammerhead ribozymes disclosed herein.

In some aspects, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also further include other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The formulation may be frozen until ready for use and then thawed and administered.

The compositions disclosed herein can be administered in sufficient amounts to transfect the cells of a desired tissue and to provide sufficient levels of hammerhead ribozymes without undue adverse effects. In some aspects, acceptable routes of administration include, but are not limited to, direct delivery to the selected organ (e.g., injection into the eye), oral, intravenous, intramuscular, subcutaneous, intradermal, intratumoral, and other parental routes of administration. In some aspects, the route of administration can be by direct injection. In some aspects, the route of administration can be by intravenous delivery. In some aspects, the compositions are administered intraocularly, intravitreally, subretinally, or suprachoroidally. Routes of administration can be combined, if desired.

An effective amount of the viral vector is an amount sufficient to target infect an animal, target a desired tissue. The effective amount will depend primarily on factors such as the species, age, weight, health of the subject, and the tissue to be targeted, and may thus vary among animal and tissue.

Formulation of pharmaceutically-acceptable excipients and carrier solutions are well-known to those of skill in the art, as is the development of suitable dosing and treatment regimens for using the particular compositions described herein in a variety of treatment regimens.

In some aspects, these formulations can contain at least about 0.10% of the active compound or more, although the percentage of the active ingredient(s) may, of course, be varied and can be conveniently be between about 1 or 2% and about 70% or 80% or more of the weight or volume of the total formulation. Naturally, the amount of active compound in each therapeutically-useful composition can be prepared in such a way that a suitable dosage can be obtained in any given unit dose of the compound. Factors such as solubility, bioavailability, biological half-life, route of administration, product shelf life, as well as other pharmacological considerations can be contemplated by one skilled in the art of preparing such pharmaceutical formulations, and as such, a variety of dosages and treatment regimens can be desirable.

In some aspects, it will be desirable to deliver the AAV-based therapeutic constructs in suitably formulated pharmaceutical compositions as disclosed herein either subcutaneously, intrapancreatically, intranasally, intracardiacally, parenterally, intravenously, intramuscularly, orally, intraperitoneally, by inhalation, or intraocularly. In some aspects, the administration modalities as described in U.S. Pat. Nos. 5,543,158; 5,641,515 and 5,399,363 (each specifically incorporated herein by reference in its entirety) can be used to deliver AAVs. In some aspects, a preferred mode of administration can be intraocularly or intravitreally, conjunctivally, subretinally, suprachoroidally, intracamerally, or intracorneally.

The pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form can be sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride can be included. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions can be suitable for intravenous, intramuscular, subcutaneous, and intraperitoneal administration. In this connection, a sterile aqueous medium that can be employed will be known to those of skill in the art. For example, one dosage can be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions can be prepared by incorporating the active AAV or lentivirus in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

The compositions disclosed herein can be also be formulated in a neutral or salt form. Pharmaceutically-acceptable salts, include the acid addition salts (formed with the free amino groups of the protein) and which can be formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations can be easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the AAV or lentiviral vector delivered hammerhead ribozymes can be formulated for delivery either encapsulated in a lipid particle, a liposome, a vesicle, a nanosphere, or a nanoparticle or the like.

Such formulations can be used for the introduction of pharmaceutically acceptable formulations of the nucleic acids constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes can be formed from phospholipids that can be dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core. Alternatively, nanocapsule formulations of the AAV or lentivirus can be used. Nanocapsules can generally entrap substances in a stable and reproducible way. To avoid side effects due to intracellular polymeric overloading, such ultrafine particles (sized around 0.1 p.m.) should be designed using polymers able to be degraded in vivo. Biodegradable polyalkyl-cyanoacrylate nanoparticles that meet these requirements are contemplated for use.

In some aspects, the methods can include administering one or more additional therapeutic agents to a subject who has been administered any of pharmaceutical composition as described herein.

The nucleotides disclosed herein can be subject to various changes, substitutions, insertions, and deletions where such changes provide for certain advantages in its use. In some aspects, the nucleotides disclosed herein that bind to an mRNA target sequence can be substantially homologous with, rather than be identical to, the sequence of a recited nucleotide where one or more changes are made and it retains the ability to function as specifically binding to and/or complexing with mRNA target sequence, wherein the mRNA target of interest comprises a NUH cleavage site.

The nucleotides disclosed herein can also include functional equivalents of the nucleotides described herein. As used herein, the term “functional equivalents” can refer to nucleic acid sequence variants having a nucleic acid substitution, addition, or deletion in some of the nucleic acid sequence of the nucleotide while simultaneously having similar or improved biological activity, compared with the nucleotide as described herein. In some aspects, the nucleic acid deletion, substitution or addition can be located in a region that is not directly involved in the activity of the nucleotide disclosed herein.

In some aspects, the nucleic acid sequence of the hammerhead ribozymes disclosed herein can include a nucleic acid sequence that has substantial identity to any of the sequences of the nucleotides disclosed herein. As used herein, the term “substantial identity” means that two nucleic acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the nucleic acid sequence of the nucleotides disclosed herein can include a nucleotide sequence that has some degree of identity or homology to any of sequences of the nucleotides disclosed herein. The degree of identity can vary and be determined by methods known to one of ordinary skill in the art. The terms “homology” and “identity” each refer to sequence similarity between two nucleotide sequences. Homology and identity can each be determined by comparing a position in each sequence which can be aligned for purposes of comparison. When a position in the compared sequence is occupied by the same nucleic acid residue, then the nucleotides can be referred to as identical at that position; when the equivalent site is occupied by the same amino acid (e.g., identical) or a similar nucleic acid (e.g., similar in steric and/or electronic nature), then the molecules can be referred to as homologous at that position. A percentage of homology or identity between sequences is a function of the number of matching or homologous positions shared by the sequences. The peptides described herein can have at least or about 25%, 50%, 65%, 75%, 80%, 85%, 90%, 95%, 96%, 97%, 98%, or 99% identity or homology to any of the hammerhead ribozyme nucleic acid sequences disclosed herein.

As discussed herein, there are numerous variants of the nucleotide that bind mRNA target sequences that are known and herein contemplated. Nucleotides variants and derivatives are well understood to those of skill in the art and in can involve nucleic acid sequence modifications. For example, nucleic acid sequence modifications typically fall into one or more of three classes: substitutional, insertional or deletional variants. Insertions include nucleotide and/or carboxyl terminal fusions as well as intrasequence insertions of single or multiple nucleic acid residues. Insertions ordinarily will be smaller insertions than those of amino or carboxyl terminal fusions, for example, on the order of one to four residues. Deletions are characterized by the removal of one or more nucleic acid residues from the nucleotide sequence. Typically, no more than about from 2 to 6 residues are deleted at any one site within the nucleotide. Nucleic acid substitutions are typically of single residues, but can occur at a number of different locations at once; insertions usually will be on the order of about from 1 to 10 nucleic acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Substitutional variants are those in which at least one residue has been removed and a different residue inserted in its place.

Labels. The peptides described herein can further comprise one or more labels or detection tags (e.g., FLAG™ tag, epitope or protein tags, such as myc tag, 6 His, and fluorescent fusion protein). In some aspects, the linker can be Fc or albumin. In some aspects, the label (e.g., FLAG™ tag) can be fused to the peptide. In some aspects, the disclosed methods and compositions can further comprise a fusion protein, or a polynucleotide encoding the same. In various aspects, the fusion protein comprises at least one epitope-providing amino acid sequence (e.g., “epitope-tag”), wherein the epitope-tag can be selected from i) an epitope-tag added to the N- and/or C-terminus of the peptide; or ii) an epitope-tag inserted into a region of the peptide, and an epitope-tag replacing a number of amino acids in the peptide.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In some aspects, the disclosed methods and compositions comprise an epitope-tag wherein the epitope-tag has a length of between 6 to 15 amino acids. In some aspects, the epitope-tag can have a length of 9 to 11 amino acids. The disclosed methods and compositions can also comprise a fusion protein comprising two or more epitope-tags, either spaced apart or directly in tandem. Further, the disclosed methods and composition can comprise 2, 3, 4, 5 or even more epitope-tags, as long as the fusion protein maintains its biological activity/activities (e.g., “functional”).

In some aspects, the epitope-tag can be a VSV-G tag, CD tag, calmodulin-binding peptide tag, S-tag, Avitag, SF-TAP-tag, strep-tag, myc-tag, FLAG-tag, T7-tag, HA (hemagglutinin)-tag, His-tag, S-tag, GST-tag, or GFP-tag. The sequences of these tags are described in the literature and well known to the person of skill in art.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

The construction of fusion proteins from domains of known proteins, or from whole proteins or proteins and peptides, is well known. Generally, a nucleic acid molecule that encodes the desired protein and/or peptide portions are joined using genetic engineering techniques to create a single, operably linked fusion oligonucleotide. Appropriate molecular biological techniques can be found in Sambrook et al. (Molecular Cloning: A laboratory manual Second Edition Cold Spring Harbor Laboratory Press, Cold spring harbor, NY, USA, 1989). Examples of genetically engineered multi-domain proteins, including those joined by various linkers, and those containing peptide tags, can be found in the following patent documents: U.S. Pat. No. 5,994,104 (“Interleukin-12 fusion protein”); U.S. Pat. No. 5,981,177 (“Protein fusion method and construction”); U.S. Pat. No. 5,914,254 (“Expression of fusion polypeptides transported out of the cytoplasm without leader sequences”); U.S. Pat. No. 5,856,456 (“Linker for linked fusion polypeptides”); U.S. Pat. No. 5,767,260 (“Antigen-binding fusion proteins”); U.S. Pat. No. 5,696,237 (“Recombinant antibody-toxin fusion protein”); U.S. Pat. No. 5,587,455 (“Cytotoxic agent against specific virus infection”); U.S. Pat. No. 4,851,341 (“Immunoaffinity purification system”); U.S. Pat. No. 4,703,004 (“Synthesis of protein with an identification peptide”); and WO 98/36087 (“Immunological tolerance to HIV epitopes”).

The placement of the functionalizing peptide portion (epitope-tag) within the subject fusion proteins or peptides can be influenced by the activity of the functionalizing peptide portion and the need to maintain at least substantial fusion protein, such as TCR, biological activity in the fusion. Two methods for placement of a functionalizing peptide are: N-terminal, and at a location within a protein portion that exhibits amenability to insertions. Though these are not the only locations in which functionalizing peptides can be inserted, they serve as good examples, and will be used as illustrations. Other appropriate insertion locations can be identified by inserting test peptide encoding sequences (e.g., a sequence encoding the FLAG peptide) into a construct at different locations, then assaying the resultant fusion for the appropriate biological activity and functionalizing peptide activity, using assays that are appropriate for the specific portions used to construct the fusion. The activity of the subject proteins can be measured using any of various known techniques, including those described herein.

In some aspects, any of the nucleotides or compositions disclosed herein can further include imaging agents. In some aspects, an imaging agents can include any substance that can be used for imaging or detecting a region of interest (ROI) in a subject and/or diagnosing the presence or absence of a disease or diseased tissue in a subject. The imaging agent can be used to generate a signal, which can be measured and whose intensity can related, and, in some aspects, be proportional, to the distribution of the imaging agent and activated platelets in the subject. Examples of imaging agents include, but are not limited to radionuclides, fluorescent dyes, chemiluminescent agents, colorimetric labels, and magnetic labels. In some aspects, the imaging agent can include a radiolabel that can be detected using gamma imaging wherein emitted gamma irradiation of the appropriate wavelength is detected. Methods of gamma imaging include, but are not limited to, SPECT and PET. For SPECT detection, the chosen radiolabel can lack a particular emission, but can produce a large number of photons in, for example, a 140-200 keV range. For PET detection, the radiolabel can be a positron-emitting moiety, such as 19F.

In some aspects, the imaging agent can include an MRS/MRI radiolabel, including but not limited to gadolinium, 19F, 13C, that can be coupled (e.g., attached or complexed) with the composition using general organic chemistry techniques. The imaging agent can also include radiolabels, such as 18F, 11C, 75Br, or 76Br for PET by techniques well known in the art and are described by Fowler, J. and Wolf, A. in Positron Emission Tomography and Autoradiography (Phelps, M., Mazziota, J., and Schelbert, H. eds.) 391-450 (Raven Press, NY 1986) the content of which is hereby incorporated by reference. The imaging can also include 1231 for SPECT.

In some aspects, the imaging agent can further include metal radiolabels. In some aspects, the radiolabel can be Technetium-99m (99mTc). Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents”, Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

Sequences. Sequences are shown in Table 1.

TABLE 1
Ribozyme Sequences.

| SEQ | | |
|---|---|---|
| ID | | |
| NO: | Name | Sequence |
| 28 | WT GAAA | GGGUAGAGCGUCUGAUGAGGCCGAAAGGCCGAAAG |
| | | GAAGUU |
| 98 | AZU GAAA | GGGUUGAGCGUCUGAUGAGGCCGAAAGGCCGAAAG |
| | | GAAGUU |
| 99 | A7G GAAA | GGGUGGAGCGUCUGAUGAGGCCGAAAGGCCGAAAG |
| | | GAAGUU |
| 100 | WT UUCG CAP | GGGUAGAGCGUCUGAUGAGGCCCUUGGGCCGAAAG |
| | (WT CAP | GAAGUU |
| | Variant) | |
| 101 | WT UUCG CAP | GGGUAGAGCGUCUGAUGAGGCCUUCGGGCCGAAAG |
| | (WT CAP | GAAGUU |
| | Variant) | |
| 102 | WT CUUG CAP | GGGUAGAGCGUCUGAUGAGGCCCUUGGGCCGAAAG |
| | (WT CAP | GAAGUU |
| | Variant) | |
| 2 | WT AGUA CAP | GGGUAGAGCGUCUGAUGAGGCCAGUAGGCCGAAAG |
| | (WT CAP | GAAGUU |
| | Variant) | |
| 98 | A7U GAAA CAP | GGGUUGAGCGUCUGAUGAGGCCGAAAGGCCGAAAG |
| | (A7U CAP | GAAGUU |
| | Variant) | |
| 103 | A7U GUGU CAP | GGGUUGAGCGUCUGAUGAGGCCGUGAGGCCGAAAG |
| | (A7U CAP | GAAGUU |
| | Variant) | |
| 104 | A7U CAAA CAP | GGGUUGAGCGUCUGAUGAGGCCCAAAGGCCGAAAG |
| | (A7U CAP | GAAGUU |
| | Variant) | |
| 105 | A7U UAAA CAP | GGGUUGAGCGUCUGAUGAGGCCUAAAGGCCGAAAG |
| | (A7U CAP | GAAGUU |
| | Variant) | |
| 106 | A7U AAAA CAP | GGGUUGAGCGUCUGAUGAGGCCAAAAGGCCGAAAG |
| | (A7U CAP | GAAGUU |
| | Variant) | |
| 107 | A7U GGAA CAP | GGGUUGAGCGUCUGAUGAGGCCGGAAGGCCGAAAG |
| | (A7U CAP | GAAGUU |
| | Variant) | |
| 108 | A7U GUUA CAP | GGGUUGAGCGUCUGAUGAGGCCGUUAGGCCGAAAG |
| | (A7U CAP | GAAGUU |
| | Variant) | |
| 109 | A7U GCAA CAP | GGGUUGAGCGUCUGAUGAGGCCGCAAGGCCGAAAG |
| | (A7U CAP | GAAGUU |
| | Variant) | |
| 110 | A7U GAGA CAP | GGGUUGAGCGUCUGAUGAGGCCGAGAGGCCGAAAG |
| | (A7U CAP | GAAGUU |
| | Variant) | |
| 111 | A7U GAUA CAP | GGGUUGAGCGUCUGAUGAGGCCGAUAGGCCGAAAG |
| | (A7U CAP | GAAGUU |
| | Variant) | |
| 112 | A7U GACA CAP | GGGUUGAGCGUCUGAUGAGGCCGACAGGCCGAAAG |
| | (A7U CAP | GAAGUU |
| | Variant) | |
| 113 | AZU GACA CAP | GGGUUGAGCGUCUGAUGAGGCCGAAGGGCCGAAAG |
| | (A7U CAP | GAAGUU |
| | Variant) | |
| 114 | AZU GAAC CAP | GGGUUGAGCGUCUGAUGAGGCCGAACGGCCGAAAG |
| | (A7U CAP | GAAGUU |
| | Variant) | |
| 115 | AZU GAAU CAP | GGGUUGAGCGUCUGAUGAGGCCGAAUGGCCGAAAG |
| | (A7U CAP | GAAGUU |
| | Variant) | |
| 116 | A7U AAAG CAP | GGGUUGAGCGUCUGAUGAGGCCAAAGGGCCGAAAG |
| | (A7U CAP | GAAGUU |
| | Variant) | |
| 117 | A7U AACA CAP | GGGUUGAGCGUCUGAUGAGGCCAACAGGCCGAAAG |
| | (A7U CAP | GAAGUU |
| | Variant) | |
| 118 | A7U AUUA CAP | GGGUUGAGCGUCUGAUGAGGCCAUUAGGCCGAAAG |
| | (A7U CAP | GAAGUU |
| | Variant) | |
| 119 | A7U AAUA CAP | GGGUUGAGCGUCUGAUGAGGCCAAUAGGCCGAAAG |
| | (A7U CAP | GAAGUU |
| | Variant) | |
| 120 | A7U AGCA CAP | GGGUUGAGCGUCUGAUGAGGCCAGCAGGCCGAAAG |
| | (A7U CAP | GAAGUU |
| | Variant) | |
| 1 | A7U AGUA CAP | GGGUUGAGCGUCUGAUGAGGCCAGUAGGCCGAAAG |
| | (A7U CAP | GAAGUU |
| | Variant) | |
| 121 | A7U AGUA CAP | GGGUUGAGCGUCUGAUGAGGCCUUCGGGCCGAAAG |
| | (A7U CAP | GAAGUU |
| | Variant) | |
| 122 | A7U CUUG CAP | GGGUUGAGCGUCUGAUGAGGCCCUUGGGCCGAAAG |
| | (A7U CAP | GAAGUU |
| | Variant) | |

### Pharmaceutical Compositions

As disclosed herein, are pharmaceutical compositions, comprising the hammerhead ribozymes and compositions comprising the hammerhead ribozymes described herein and a pharmaceutical acceptable carrier. In some aspects, the pharmaceutical composition can be formulated for intravenous, subcutaneous, intradermal, intraperitoneal, intraocular, intravitreal, subretinal, suprachoroidal, intracameral, intracorneal, or subconjunctival administration. The compositions of the present disclosure also contain a therapeutically effective amount of the nucleotides as described herein. The nucleotides and compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the peptides disclosed herein. Thus, compositions can be prepared for parenteral administration that includes the nucleotides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described herein can also be formulated so as to provide slow, prolonged, or controlled release. For example, a controlled-release preparation is a pharmaceutical composition capable of releasing the nucleotides or compositions disclosed herein at a desired or required rate to maintain constant activity for a desired or required period of time.

### Methods of Treatment

Disclosed herein, are methods of treating a subject with a disease caused by increased intracellular levels of a protein. In some aspects, the methods can comprise: administering to the subject a therapeutically effective amount of any of the one or more of the nucleotides or hammerhead ribozymes disclosed herein. In some aspects, the protein can be rhodopsin, peripherin-2, centrosomal, or transcription factor 4. In some aspects, the protein can be a protein encoded by an mRNA, wherein the mRNA has a NUH cleavage site.

Disclosed herein are methods of treating retinitis pigmentosa in a subject. In some aspects, the methods can comprise administering to the subject a therapeutically effective amount of any of the hammerhead ribozymes disclosed herein or the pharmaceutical composition disclosed herein.

Disclosed herein are methods of treating dry macular degeneration in a subject. In some aspects, the methods can comprise administering to the subject a therapeutically effective amount of any of the hammerhead ribozymes disclosed herein or the pharmaceutical composition disclosed herein.

Disclosed herein are methods of treating retinal dystrophies in a subject. In some aspects, the methods can comprise administering to the subject a therapeutically effective amount of any of the hammerhead ribozymes disclosed herein or the pharmaceutical composition disclosed herein. Retinal dystrophies are a group of degenerative disorders of the retina with clinical and genetic heterogeneity. Common presentations include color blindness or night blindness, peripheral vision abnormalities, and subsequent progression to complete blindness in progressive conditions.

Disclosed herein are methods of treating primary open angle glaucoma in a subject. In some aspects, the methods can comprise administering to the subject a therapeutically effective amount of any of the hammerhead ribozymes disclosed herein or the pharmaceutical composition disclosed herein.

Disclosed herein are methods of treating corneal dystrophies in a subject. In some aspects, the methods can comprise administering to the subject a therapeutically effective amount of any of the hammerhead ribozymes disclosed herein or the pharmaceutical composition disclosed herein. Corneal dystrophies are a group of rare genetic eye disorders. With corneal dystrophies, abnormal material builds up in the cornea. Corneal dystrophies can be grouped into three categories. Anterior or superficial corneal dystrophies affect the outermost layers of the cornea: the epithelium and Bowman's membrane. Stromal corneal dystrophies affect the stroma, which is the middle and thickest layer of the cornea. Posterior corneal dystrophies affect the innermost parts of the cornea: the endothelium and the Descemet membrane. The most common posterior corneal dystrophy is Fuchs' dystrophy.

Disclosed herein are methods of increasing the turnover rate of a substrate. In some aspects, the methods can comprise administering to a subject a therapeutically effective amount of any of the hammerhead ribozymes disclosed herein or the pharmaceutical composition disclosed herein. In some aspects, the substrate can be a target mRNA sequence comprising a hammerhead ribozyme cleavage site, has the nucleotide sequence of NUH↓, wherein N is any nucleotide, U is U, and H is C, A or U. In some aspects, the target mRNA sequence can encode for rhodopsin (e.g., the RHO mRNA). In some aspects, the target mRNA sequence can encode for Peripherin-2 (e.g., PRPH2 mRNA). In some aspects, the target mRNA sequence can encode for centrosomal (e.g., CEP290 mRNA). In some aspects, the target mRNA sequence can encode for transcription 4 (e.g., TCF4 mRNA). In some aspects, the substrate can be rhodopsin, peripherin-2, centrosomal, or transcription factor 4. In some aspects, the method reduces or decreases the intracellular protein level of rhodopsin, peripherin-2, centrosomal, or transcription factor 4.

In some aspects, the methods disclosed herein can comprise identifying a patient in need of treatment before the administration step.

In some aspects, the methods disclosed herein comprise administering to the subject a therapeutically effective amount of any of the nucleotides or hammerhead ribozymes disclosed herein and a pharmaceutically acceptable carrier or any of the compositions disclosed herein or any of the compositions comprising any of the nucleotides or hammerhead ribozymes disclosed herein. In some aspects, the nucleotides or hammerhead ribozymes bind a target mRNA sequence. In some aspects, the target mRNA sequence comprises a hammerhead ribozyme cleavage site, wherein the hammerhead ribozyme cleavage site has the nucleotide sequence of NUH↓, wherein N is any nucleotide, U is U, and H is C, A or U.

The pharmaceutical compositions described herein can be formulated to include a therapeutically effective amount of the nucleotides disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a disease caused by increased intracellular levels of a protein that is associated with or causing a disease or disorder.

The pharmaceutical compositions described herein can be administered to the subject (e.g., a human patient) in an amount sufficient to delay, reduce, or preferably prevent the onset of clinical disease. Accordingly, in some aspects, the patient can be a human subject or patient. In therapeutic applications, compositions can be administered to a subject (e.g., a human patient) already with or diagnosed with a disease (e.g., a disease caused by increased intracellular levels of a protein that is associated with or causing a disease or disorder) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the disease is delayed, hindered, or prevented, or the disease or a symptom of the disease is ameliorated. One or more of the symptoms can be less severe. Recovery can be accelerated in an individual who has been treated.

In some aspects, the disease caused by increased intracellular levels of a protein that is associated with or causing a disease or disorder can be any eye disease. In some aspects, the eye disease can be retinitis pigmentosa, dry macular degeneration, primary open angle glaucoma, corneal dystrophies

In some aspects, subject has an eye disease or is suspected of having an eye disease.

In some aspects, any of the methods disclosed herein can further comprise administering one or more of the following: one or more vitamin supplements, low vision rehabilitation, implanting a telescopic lens to the subject's eyes to the subject, prostaglandin analogs, beta-blockers, alpha-agonists, carbonic anhydrase inhibitors, memantine, bis(7)-tacrine, nimodipine, and mirtogenol, laser trabeculoplasty (a procedure that improves drainage of eye fluid through the spongy tissue located near the cornea, called the trabecular meshwork), and surgery (e.g., Flourouracil Filtering Surgery (trabeculectomy)), corneal transplantation surgery, gene therapy, stem cell therapy, delivering wild-type copies of the RPGR gene to the subject, implanting a device such as Argus II to the subject's eyes, a VEGFR-1 inhibitor, or a VEGFR-2 inhibitor. In some aspects, the VEGFR-1 inhibitor can be SU14813, ZM 306416, Axitinib (AG 013736), Sulfatinib, Motesanib (AMG-706), MGCD-265 analog, Linifanib (ABT-869), Cediranib (AZD2171), Cediranib Maleate, Sitravatinib (MGCD516), Foretinib (GSK1363089), Lucitanib (E3810) hydrochloride, OSI-930, Pazopanib, Dovitinib (TKI-258), Pazopanib HCl (GW786034 HCl), Regorafenib (BAY 73-4506), Regorafenib (BAY-734506) Monohydrate, Lenvatinib (E7080), ODM-203, Tivozanib (AV-951), Nintedanib (BIBF 1120), Fruquintinib (HMPL-013), Nintedanib Ethanesulfonate Salt, AEE788 (NVP-AEE788), Vatalanib (PTK787) 2HCI, KRN 633, Brivanib (BMS-540215), Brivanib Alaninate (BMS-582664), or BAW2881 (NVP-BAW2881). In some aspects, the VEGFR-2 inhibitor can be Cabozantinib (BMS-907351), Ki8751, Apatinib, Ponatinib (AP24534), Ningetinib, ZM 323881 HCl, BFH772, Toceranib phosphate, LY2874455, LY2874455, AZD2932, Altiratinib, Ki20227, PP121, Sorafenib (BAY 43-9006) tosylate, BMS-794833, Golvatinib (E7050), SU5402, RAF265 (CHIR-265), Pamufetinib (TAS-115), SKLB1002, CS-2660 (JNJ-38158471), CYC116, SU5408, Sunitinib (SU11248), PD173074, Semaxanib (SU5416), SU5204, SU5205, SU5214, SU5614, SKLB 610, Erdafitinib (JNJ-42756493), Ax-itinib (AG 013736), Anlotinib (AL3818), Cediranib (AZD2171), Cediranib Maleate, Motesanib (AMG-706), MGCD-265 analog, Lenvatinib (E7080), Linifanib (ABT-869), Regorafenib (BAY 73-4506), Regorafenib (BAY-734506) Monohydrate, Regorafenib Hydrochloride, Sitravatinib (MGCD516), Telatinib, Tivozanib (AV-951), ODM-203, Dovitinib (TK1258) Lactate, Nintedanib (BIBF 1120), Donafenib (Sorafenib D3), Sul-fatinib, Lucitanib (E3810) hydrochloride, Brivanib (BMS-540215), Brivanib Alaninate (BMS-582664), Paz-opanib HCl (GW786034 HCl), Pazopanib, Fruquintinib (HMPL-013), Vatalanib (PTK787) 2HCl, Vandetanib (ZD6474), SU14813, ENMD-2076,AEE788 (NVP-AEE788), Sorafenib (BAY 43-9006), KRN 633.

Amounts effective for this use can depend on the severity of the disease and the weight and general state and health of the subject. Suitable regimes for initial administration and booster administrations are typified by an initial administration followed by repeated doses at one or more hourly, daily, weekly, or monthly intervals by a subsequent administration. For therapeutic uses, the nucleotides and compositions can include a pharmaceutically acceptable excipient. Such compositions can be formulated without undue experimentation for administration to a mammal, including humans, as appropriate for the particular application. Additionally, proper dosages of the compositions can be determined without undue experimentation using standard dose-response protocols. For example, a subject can receive any of the nucleotides or compositions disclosed herein one or more times per week (e.g., 2, 3, 4, 5, 6, or 7 or more times per week).

The total effective amount of any of nucleotides in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month).

The therapeutically effective amount of any of the nucleotides disclosed herein present within the pharmaceutical compositions described herein and used in the methods as disclosed herein applied to mammals (e.g., humans) can be determined by one of ordinary skill in the art with consideration of individual differences in age, weight, and other general conditions (as mentioned herein).

### EXAMPLES

### Example 1: Log-Order Improved in Trans Hammerhead Ribozyme Turnover Rates: Reevaluating Therapeutic Space for Small Catalytic RNAs

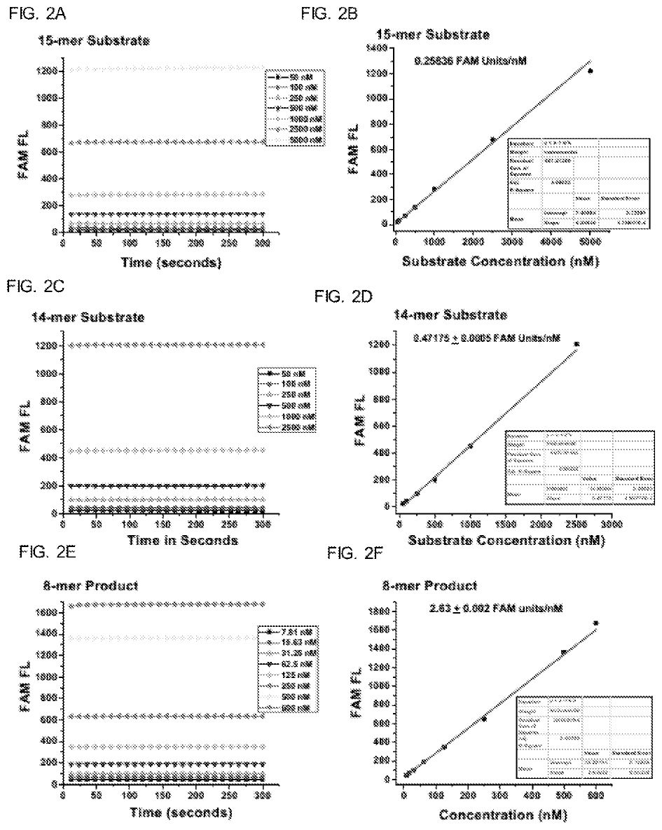

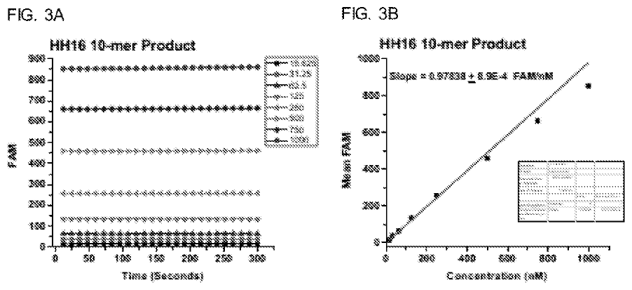

Results. High-performance hhRz kinetics assay. The 266 CUC↓ EhhRz targets a region in human RHO mRNA accessible at both the secondary and tertiary structural levels ((FIG. 1A). Described herein is a moderate-throughput hhRz fluorescence cleavage assay that does not require gels (FIG. 1B). Fluorescence from short RNA substrates with a 5′ FAM fluorophore is partially quenched by a 3′ BHQ1 quencher because of the physical proximity in the intact substrate. Biophysically, this occurs because of Forster interactions between the fluorophore and the quencher (1/r⁶ dependence, where r is the distance between 10 and 100 Å) that result in fluorescence resonance energy transfer. Site-specific cleavage of the annealed substrate by the hhRz, followed by product release and diffusion (distancing), leads to an increase in fluorescence detectable in real time on a standard real-time PCR machine with optical detectors (e.g., silicon photodiodes) (FIG. 1C). The amount of FAM fluorescence signal is linear with respect to the substrate concentration for 15-mer and 14-mer substrates and for the 8-mer fluorescent 5′ product (FIG. 2); each cleaved and released substrate molecule is expected to contribute linearly to the fluorescence emission signal, and 8-mer product fluorescence is estimated at 3.11 FAM U nM⁻¹. Initial rates were estimated by fitting a line by eye to the initial data points in the time-dependent fluorescence emission data, which corresponded to rates estimated by linear least-squares fitting, thereby validating the data reduction process (FIG. 1C). The 15-mer substrate alone showed steady background fluorescence over time. EhhRzs harboring the wild-type stem-loop II tetraloop [WT(GAAA)], and those harboring the altered A7 residue (A7U) with either GAAA or AGUA tetraloop [A7U(GAAA) or A7U(AGUA), respectively] had progressively faster rates of turnover originating from the substrate-alone baseline. The positive slopes are the result of catalytic turnover, given that catalytic core mutations (G5C, G8C, G12C, independent or aggregated mutations) known to prevent catalysis obviate optical turnover measure [here shown for the A7U(GAAA) construct]. Also, substrates with a non-cleavable CUG triplet showed no evidence of turnover. HH16, a well-studied mhhRz with a single-turnover rate of up to 1 min⁻¹ at supraphysiological Mg²⁺ levels (>10 mM) and lower temperature (−25° C.) demonstrated a slow steady-state cleavage rate in this assay with a labeled 18-mer substrate over a 5-min reaction under conditions of 0.5 mM Mg²⁺ and 37° C.; the lower level of baseline fluorescence with the HH16 reaction is likely an index of quenching as a result of substrate structure (see below). Plotting of HH16 product fluorescence versus the concentration gave an estimated slope of 0.98 FAM U nM-1 (FIG. 3). Estimated turnover rate, corrected for product fluorescence and the optical effects of substrate binding, is approximately 9 nM min⁻¹ which is on the same scale of reports of this enzyme (1-2/min)

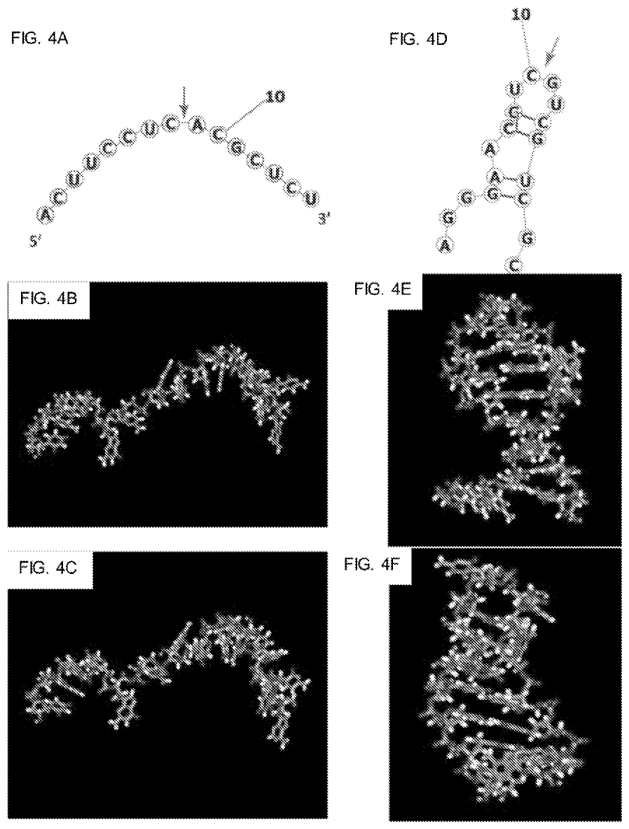

The active 266 RHO EhhRzs in this study drove substrate cleavage to completion. In classic gel-based assays, the EhhRzs cleave 15-mer and 14-mer substrates to yield the quantitative conversion of substrate to products within a 5-min reaction time; the products co-migrate and are observable as a single band on PAGE urea gels when imaged for FAM and then post-stained and imaged with SYBR Gold (RNA backbone fluorophore) (FIG. 1D). These PAGE results validate this moderate-throughput optical assay. The enzyme core of the EhhRzs developed here was based on the enzyme core, stem-loop II, and cap (GAAA) of the HH16 enzyme. The data show that the 15-mer 266 RHO substrate has no secondary or tertiary structure in predictive algorithms (MFold and RNA Composer; FIGS. 4A-C). By contrast, the HH16 substrate (18-mer) has strong secondary structure (single structure predicted with ΔG=−1.3 kCal mol⁻¹; FIGS. 4D-F) and does not possess a U7 residue 3′ of the GUC↓ cleavage motif.

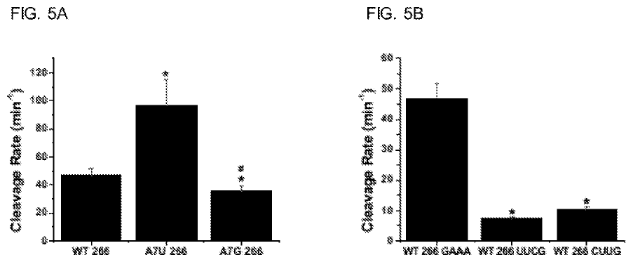

U7 substrate residue of the “uncompromised” mhhRz is not important. Evidence from natural occurring extended hhRzs (xhhRz) acting in cis and engineered hhRzs designed for in trans function indicates that the stem II loop of hhRz interacts with the upstream region of hhRz while bound to its downstream substrate flank. The expected interaction between the upstream-bound antisense flank and the loop capping stem II of the hhRz core was investigated. In RHO mRNA there is a U residue at the seventh nucleotide position (underlined) downstream of the substrate cleavage site after position 266 (5′-ACUUCCUC↓ACGCUCU-3′ (SEQ ID NO: 17). Based on the Schistosomal xhhRz structure, O'Rourke et al. (2015) proposed an unpaired U7 residue forming a Hoogsteen base pair interaction with the fourth nucleotide of the GUGA stem 11 cap in their hhRz: substrate complex. They speculated that this interaction is essential and sufficient to generate high hhRz enzyme activity, specifically in their assays under enzyme excess (pre-annealed) and high Mg²⁺ (10 mM) conditions. As described herein, under multi-turnover conditions ([S]/[E]=10:1 [1 μM substrate to 0.1 μM EhhRz 266]) and at physiological intracellular concentrations of free Mg²¹ (0.5 mM) and temperature (37° C.), WT EhhRz 266 showed strong levels of activity (mean±SD): 46.48±5.42 (SD) min⁻¹, n=36), greater than the rates expected for an mhhRz (FIG. 5A). It was tested whether the U7 residue of the substrate, being paired with a Watson-Crick complementary “A7” on the upstream antisense flank of the mhhRz, is partially constrained from forming a Hoogsteen face base pairing with the A₄ residue of the GAAA stem II loop. An A7U mutation in the upstream antisense flank of the EhhRz eliminates this constraint on the substrate U7 residue by obviating the Watson-Crick interaction. The turnover rate of A7U EhhRz 266 was significantly enhanced relative to that of the WT enzyme (96.84±18.21 min⁻¹, n=52). By contrast, when the Watson-Crick base at the same position is maintained with an A7G mutation in EhhRz (U:A has free energy similar to that of a U:G base pair), the rate was significantly decreased relative to those of both the WT and A7U EhhRzs (35.55±3.96 min⁻¹, n=23). Statistical comparison by one-way ANOVA across the samples showed significant differences (p=3.86 E-41). Bonferroni testing showed the turnover rates of A7U (p=1.59E-33) and A7G (p=0.006) EhhRzs were significantly different from that of the WT and from each other (p=2.16E-35).

With the substrate U7 paired to the complementary “A7” in the WT EhhRz (46.48±5.42 min⁻¹, n=36), the GAAA tetraloop capping stem II was mutated to a highly stable UUCG tetraloop, which dramatically reduced turnover activity (7.32±0.46 min⁻¹, Bonferroni p=2.08E-26 versus WT) (FIG. 5B). An alternative CUUG had a similar impact (10.38±0.86 min⁻¹, Bonferroni p=7.55 E-25 versus WT); the turnover rates of EhhRzs with UUCG and CUUG tetraloops were not significantly different (p=0.57). These outcomes confirm that stem II loop interacts with the upstream region of the hhRz in the EhhRz format (O'Rourke et al., 2015; Khvorova et al. 2003). The physical chemical properties (e.g., energy and structure) of the Watson-Crick base pair of the hhRz with the substrate U7 residue influence the interaction with stem-loop II, and this substantially impacts the turnover rate. The interaction is optimized when the U7 substrate residue is not base paired to the EhhRz.

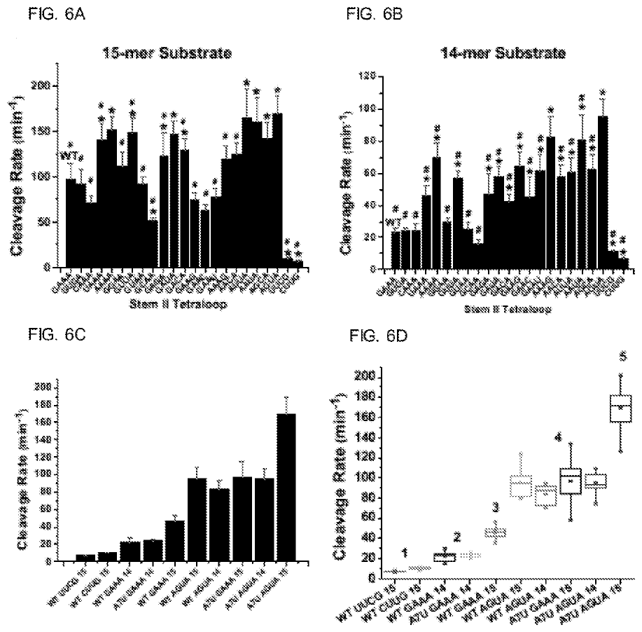

To further investigate the stem II loop interaction, the stem IT cap sequence was varied from the wild type (“WT”) 4-nt GAAA tetraloop to a variety of alternatives in the A7U EhhRz (23 total; FIG. 6A). The standard 15-mer substrate was used under multi-turnover conditions ([S][E]; 1:0.1 μM) at physiological [Mg²⁺ ](0.5 mM) and temperature (37° C.) in 10 mM Tris-HCl (pH 7.5). The WT loop is a standard GNRA tetraloop (where N is any nucleotide and R is a purine), and five of the constructs were of this variety. The A7U EhhRz with the GAAA tetraloop had a turnover rate of approximately 97 min⁻¹. The best tetraloops had turnover rates greater than 150 min⁻¹ and were AGUA (169.32±19.86 min⁻¹), AUUA (164.92±31.71 min⁻¹), AAUA (159.52±27.80 min⁻¹), and AAAA (152.40±13.71 min⁻¹). The AUUA, AAUA, and AAAA constructs had means that were not significantly different from AGUA (FIG. 6A); the AGUA construct was used for more detailed studies. Although this screen was not nearly an exhaustive search (9% of 256 [4⁴] possibilities), the outcomes demonstrate a wide range of rates with both strong enhancement and suppression of an already enhanced turnover rate observed with the WT A7U EhhRz 266: the minimum with CUUG (6.63±0.85 min⁻¹)<WT(GAAA) (96.84±18.21 min⁻¹)<maximum with AGUA (169.32±19.86 min⁻¹). A one-way ANOVA refuted the null hypothesis of equivalence across the ensemble of constructs (F=70.79, p=1.225 E-114). Internal pairwise comparisons to the WT(GAAA) construct with parametric and nonparametric tests showed significant differences (p<0.05, Bonferroni testing, asterisks) between the WT tetraloop (GAAA) and the alternatives, as evaluated by multiple statistical testing comparisons. These results show the extensive effect of varying the stem II capping tetraloop on EhhRz catalytic activity (13 of the 22 tetraloop variants [59%] were significantly different than the GAAA control, with some differences exceeding log-order). Statistical comparison of the EhhRz with the AGUA tetraloop found statistical significance (#, Bonferroni, p<0.05) in 17 of 22 variants (77.3%). The set of tetraloops of EhhRzs with turnover activity not statistically different from AGUA were AAAA, AUUA, AAUA, AGCA, and GAUA.

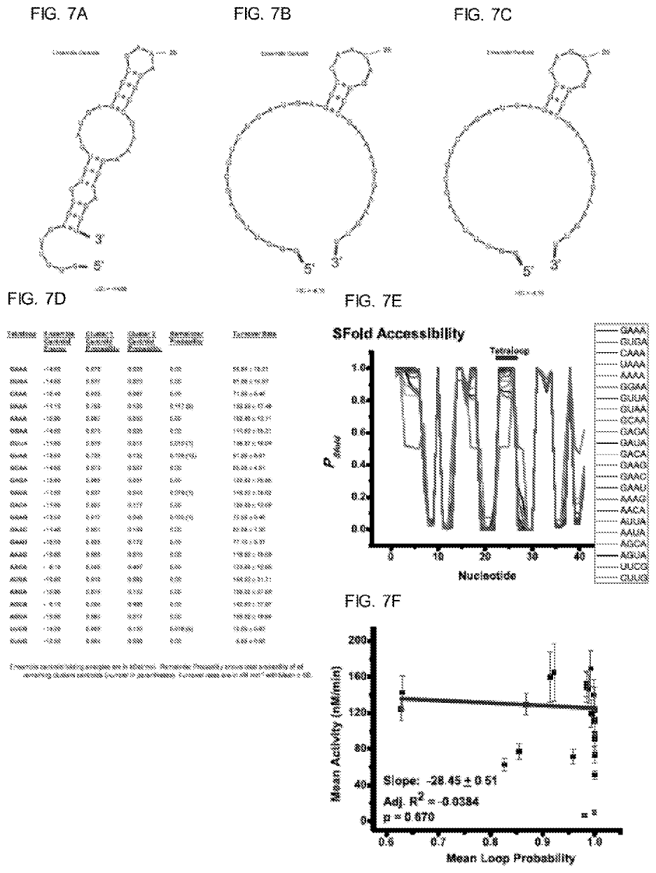

It was also investigated whether the variation in turnover rates was related to structural folding parameters of the individual EhhRzs with varying Stem-II tetraloops but otherwise constant sequence compositions. Using the statistical RNA folding environment, SFold, and the program (Srna), the overall ensemble centroid, cluster centroids, and sstrand vector output for the “WT” tetraloop (GAAA, found in HH16) and the other (n=22) tetraloops evaluated in the sample was obtained (FIG. 7). The overall ensemble centroids, represented by GAAA, had the same structure (FIG. 7A) except for AACA FIG. 7B) and AGCA (FIG. 7C) which have more open ensemble centroid structures with the last G:C base pair in stem II unformed. The high probability cluster centroids (including those of AACA and AGCA) have the same structure as the GAAA ensemble centroid structure (FIG. 7A). Free energies of the ensemble structures and probabilities of the centroid structures are tabulated with the turnover rates against the 15-mer substrate (FIG. 7D). The sstrand maps of accessibility show the tetraloop sequence having high probability of being open single stranded structure as expected for the elements sampled including the AACA and AGCA variants, for which the ensemble centroid loop was predicted to be larger (FIG. 7E). Overall predicted structure was well conserved over the set of tested EhhRzs targeting the 266 CUC↓ cleavage site. A comparison of the average accessibility over the tetraloop sequence versus the turnover rate did not show a significant linear relationship (FIG. 7F). There were high probability loops that showed both very low (e.g., UUCG) and very high turnover rates (e.g., AGUA), and medium probability loops that also showed high rates. In summary, there was no systematic EhhRz folding variation or local tetraloop accessibility that corresponded with variation in measured turnover rates for the ensemble tested. The antisense flank interaction seen in most ensemble centroids and the dominant cluster centroids cannot be a deterrent to annealing and cleavage at 37° C. given the robust rates measured experimentally. As the nucleotide composition of the EhhRz ensemble was otherwise identical except for the Stem II tetraloop, it was concluded that the nucleotide composition of the tetraloop (L2.1 through L2.4, FIG. TA) was the driving feature of the variation in turnover rates.

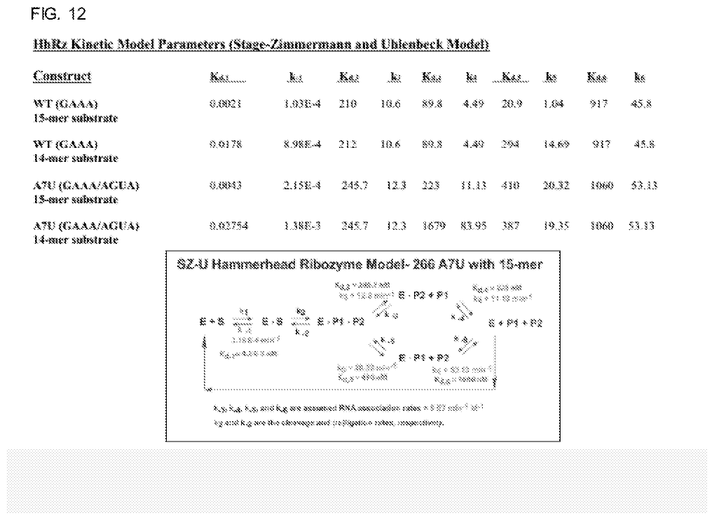

To determine the extent to which the U7 residue impacts rate enhancement, the substrate to 14 nt was shorted by deleting the U7 residue identified as important to the interactions. This change would also increase the product leaving rate of one antisense flank (FIG. 12), which is expected to enhance the turnover rate if product release is rate limiting at 37° C. in the overall mechanism (Stage-Zimmermann and Uhlenbeck, 1998). Using the same set of cap tetraloop variants, a similar activity landscape was surprisingly found (FIG. 6B). The four best tetraloops had high turnover rates: AGUA (95.15±10.89 min⁻¹), AAAG (82.78±12.90 min⁻¹), AAUA (80.70 T 15.61 min⁻¹), and AAAA (69.85±8.84 min⁻¹), which were significantly greater than GAAA. Rates with the 14-mer were uniformly lower than with the 15-mer substrate under identical reaction conditions: minimum with CUUG (6.59±0.71 min⁻¹)<GAAA (23.42±2.27 min⁻¹)<AGUA (95.15±10.89 min⁻¹). A one-way ANOVA refuted the null hypothesis of equivalence across the ensemble of constructs (F=98.04, p=2.35E-120). Internal pairwise comparisons to the WT(GAAA) construct showed significant differences (p<0.05, Bonferroni testing) for many constructs (asterisks). The tetraloop of the EhhRz with turnover activity not statistically different from that with AGUA was AAAG (p=0.56). These results show the extensive effect of varying the stem II capping tetraloop on EhhRz catalytic activity (17 of the 22 tetraloop variants [77%] were significantly different than the GAAA control).

The best tetraloop performers were AGUA and AAAG for the 14-mer and AGUA and AUUA for the 15-mer substrates, respectively. The CUUG tetraloop yielded the lowest turnover rate for both 14-mer and 15-mer substrates. With the same capping tetraloop, predicted improvements in the leaving rate for the 14-mer substrate were expected to enhance the overall turnover rate rather than decrease it. Calculations of the equilibrium constants and rates for the hhRz reaction scheme, by way of an established nearest-neighbor model approach, confirmed this (FIG. 12). For a given substrate, the enhancement or suppression of turnover rate must relate to structural and chemical interactions that drive the formation of the catalytically active state(s) leading to target cleavage (k₂). Overall, major findings are that the composition of the tetraloop has a marked impact on kinetic turnover rate, and that the U7 residue of the substrate is not essential to enhanced rates, in contrast to the state of the art that it was necessary and sufficient. In the absence of the U7 in the substrate, a U7 residue in the hhRz (i.e., A7U) yields greatly enhanced rates that vary with different stem II tetraloops.

Next, the kinetic outcomes between the 15-mer and 14-mer substrates were compared with the WT(GAAA), A7U(GAAA), and A7U(AGUA) EhhRzs (FIG. 6C); one-way ANOVA revealed that the differences between the means were significant (F=283.31, p=8.80 E-106). The addition of A7U to the WT EhhRz (GAAA) (non-pairing U7 substrate) enhanced the rate for the 15-mer substrate. The AGUA tetraloop enhanced the activity relative to that of the WT construct (GAAA) when the U7 of the substrate was either base paired (15-mer) or absent (14-mer) and further enhanced the A7U EhhRz variation with the 15-mer substrate. The activity level of WT EhhRz (GAAA) with the 14-mer was approximately one-half that of WT EhhRz (GAAA) with the 15-mer substrate, but the A7U shift did not enhance activity of the EhhRz (GAAA) with the 14-mer substrate; the A7U shift did enhance with the AGUA tetraloop acting on the 14-mer substrate. Four constructs (WT AGUA [15-mer], WT AGUA [14-mer], A7U GAAA [15-mer], and A7U AGUA [14-mer]) generated similar levels of activity [overall ANOVA p=0.03132 with the significant difference in the internal comparisons was WT AGUA [14-mer] vs. A7U(GAAA) [15-mer](p=0.021) (this group is cluster 4 in FIG. 6D). The A7U GAAA [15-mer]) was approximately double the activity level of the WT GAAA (15-mer) reaction. The maximum turnover level was achieved with the A7U(AGUA) EhhRz and the 15-mer substrate, which was approximately double the level for the four others that appear to aggregate in activity. This finding shows that the activity enhancements may be quantized over at least five levels (the numbers above clusters (FIG. 6D)); the UUCG/CUUG constructs have approximately one-half of the activity level of other constructs at lower levels (WT(GAAA) [14-mer] and A7U(GAAA) [14-mer]). Without the A7U EhhRz variation, the AGUA-containing constructs are agnostic to the length of the substrates. The A7U variation at the 5′ end of EhhRz sensitizes the turnover rate to the length of the substrate. A7U generates an unpaired (unconstrained) U residue in the 5′ end of the EhhRz, leaving the U7 at the 3′ end of the substrate unpaired. The 5′ end of the EhhRz and the 3′ end of the substrate might act in independent or cooperative manners in the local structural space (e.g., atomic level interactions with preferential stem II loops) to influence catalytic performance.

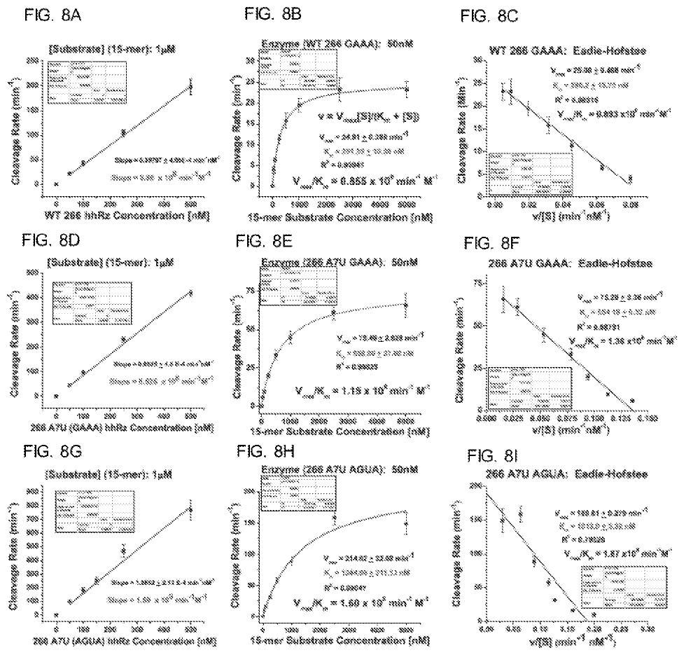

Kinetic analysis of 266 EhhRzs. A detailed analysis of the kinetic properties of the WT(GAAA), A7U(GAAA), and A7U(AGUA) 266 RHO EhhRzs was conducted. Using consistent amounts of 15-mer substrate (1 M), increasing the amount of enzyme (0-500 nM) lead to a proportional increase in the rate of the reaction measured optically under the conditions of physiological 0.5 mM Mg²⁺ and 37° C. (FIG. 8). Linear fits of the turnover rate to enzyme concentration gave statistically reliable functions, consistent with Michaelis-Menten enzyme function, with R² values of 0.99963 (ANOVA p=1.96E-6) for WT(GAAA) EhhRz 266 (FIG. 8A), 0.9977 (ANOVA p=3.04E-5) for A7U(GAAA) (FIG. 8D), and 0.99328 (ANOVA p=1.08E-5) for A7U(AGUA) (FIG. 8G). Similarly, when the y intercept was fitted according to enzyme concentration, there was also a linear relationship as expected. Subsequently, a formal Michaelis-Menten analysis was conducted for the three 266 hhRzs by increasing the substrate concentration but fixing the EhhRz concentration at 50 nM (linear range) under conditions with 0.5 mM Mg²⁺ at 37° C. The WT(GAAA) enzyme data fit well with the Michaelis-Menten hyperbolic function (ANOVA p=1.80E-11) (FIG. 8B), and a fit of the Eadie-Hofstee linear transform confirmed similar parameters (ANOVA p=7.97E-6) (FIG. 8C). The A7U(GAAA) and A7U(AGUA) kinetics were also well fit by the hyperbolic nonlinear function (ANOVA p=2.41E-12 and 4.61E-12; FIGS. 8E and 8H, respectively), with similar linear transform results (ANOVA p=3.47E-6 and 0.002; FIGS. 8F and 8I, respectively). The kinetics results of these measures for the three 266 enzymes are shown (Table 2). Enzyme efficiency (Vₘₐₓ/Kₘ) or turnover is commonly used to compare different catalytic entities. Notably, the enzyme efficiencies of the 266 EhhRzs are on the same scale as RNase A (1.38×10⁸ min⁻¹ M⁻¹), which uses the same mechanism for RNA cleavage. In particular, A7U(AGUA) has a Vₘₐₓ=214.62 min⁻¹, Kₘ=1344 nM, and Vₘₐₓ/Kₘ=1.60×10⁸ M⁻¹ min⁻¹.

TABLE 2
Michaelis-Menten Parameters for EhhRzs.

| | Vₘₐₓ | Kₘ | Vₘₐₓ/Kₘ |
|---|---|---|---|
| Construct | (min⁻¹)* | (nM)* | (min⁻¹M⁻¹) |
| WT (GAAA) | 24.91 ± 0.36 | 291.39 ± 10.06 | 0.855 × 10⁸ |
| A7U (GAAA) | 75.49 ± 2.63 | 658.80 ± 37.00 | 1.15 × 10⁸ |
| A7U (AGUA) | 214.62 ± 22.92 | 1344.08 ± 211.13 | 1.60 × 10⁸ |

*Vₘₐₓ and Kₘ parameters were obtained by nonlinear curve fitting of the Michaelis-Menten (hyperbolic) function. Variances are reported as Standard Deviations. The assays were conducted at concentration of 50 nM EhhRz.

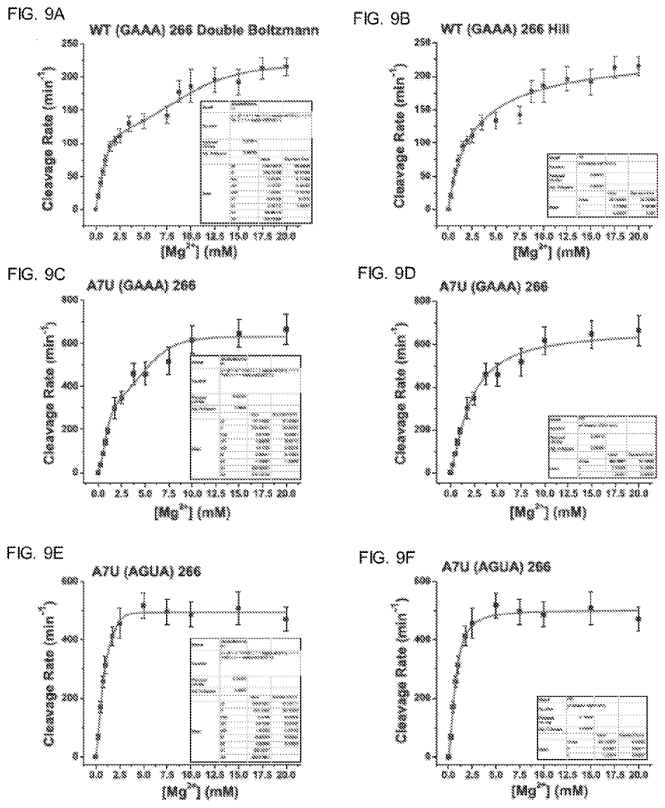

Mg²⁺ dependence of enhanced 266 hhRzs. The sensitivity of the turnover rate under [S][E] conditions (1: 0.1 M) by varying the Mg²⁺ concentration (0-20 mM) in the reaction buffer (pH 7.5 [physiological]) for the WT(GAAA), A7U(GAAA), and A7U(AGUA) 266 EhhRzs was investigated (FIG. 9). Next, two types of functionality was assessed: double Boltzmann and Hill functions. The double Boltzmann (equation 1) represents two Mg²⁺ binding sites, each with their own independent binding equilibrium.

$${k}_{obs} = {k}_{0} + {V}_{max} \times \{ ( \frac{frac}{1 + exp ( [ Mg ] - {[ Mg ]}_{0.5 , 1} ) / {k}_{1}} ) + ( \frac{frac}{1 + exp ( [ Mg ] - {[ Mg ]}_{0.5 , 2} ) / {k}_{2}} ) \} , \qquad ( EQ 1 )$$

where k₁ and k₂ are the sensitivity factors for the two titrations, [Mg]_{0.5,1} and [Mg]_{0.5,2} are the half points for the independent titrations, and frac is the fraction of the overall fit attributed to the first (most sensitive) titration. The Hill function (Equation 2) represents one or more cooperative Mg²⁺ binding sites:

$${k}_{obs} = {V}_{max} \frac{{[ Mg ]}^{n}}{{[ Mg ]}^{n} + {[ Mg ]}_{0.5}^{n}} , \qquad ( EQ 2 )$$

where [Mg]_{0.5}ⁿ is the half point for the titration, and n is the cooperativity constant.

For the three EhhRzs, the turnover rate (k_{obs}) showed a clear saturable Mg²⁺ sensitivity. The WT(GAAA) and A7U(GAAA) constructs showed clear bimodal titrations by inspection of the datasets. The independent site model fit the data for the WT(GAAA) (FIG. 9A), A7U(GAAA) (FIG. 9C), and A7U(AGUA) (FIG. 9E) EhhRzs. The Mg²⁺ isotherms were also well fit by the Hill function for the WT(GAAA) (FIG. 9B), A7U(GAAA) (FIG. 9D), and A7U(AGUA) (FIG. 9F) EhhRzs. The maximum rate (Vₘₐₓ), the Kₘ ([Mg²⁺]_{0.5}) values, and the Hill coefficients (n) varied with each construct (Table 3). For the WT(GAAA), A7U(GAAA), and A7U(AGUA) EhhRzs, the most sensitive titrations ([Mg²⁺]_{0.5} values in double Boltzmann fitting) ranged from ˜0.1 to ˜0.7 mM, and the less sensitive titrations ranged from ˜1.5 to 7.5 mM; the fractional weight of the two component titrations was approximately 50-60% for these EhhRzs. The sensitivity values for the Hill fitting for Mg²⁺ ranged from approximately 0.7 to 2.7 mM, with Hill coefficients between 0.9 (WT(GAAA) and 1.7 (A7U(AGUA)). The Vₘₐₓ value of the A7U(AGUA) construct is smaller than that of the A7U(GAAA) EhhRz, demonstrating that the AGUA tetraloop drives highly efficient Mg²⁺-dependent formation of the most catalytically active states of the EhhRz.

TABLE 3
Mg²⁺ Sensitivity Parameters for EhhRzs from Model Fitting

| Construct | Vₘₐₓ | frac | [Mg]_{0.5, 1} | k₁ | [Mg]_{0.5, 2} | k₂ | [Mg]_{0.5} | n | AdjR² |
|---|---|---|---|---|---|---|---|---|---|
| WT | 319.7 ± | 0.62 ± | 0.12 ± | −0.61 ± | 7.56 ± | −3.48 ± | | | 0.99783 |
| (GAAA) | 55.30 | 0.17 | 0.35 | 0.19 | 2.17 | 1.89 | | | |
| Two Site | | | | | | | | | |
| WT | 237.53 ± | | | | | | 2.74 ± | 0.91 ± | 0.98254 |
| (GAAA) | 18.76 | | | | | | 0.59 | 0.081 | |
| Hill | | | | | | | | | |
| A7U | 708.39 ± | 0.48 ± | 0.70 ± | −0.40 ± | 4.42 ± | −1.74 ± | | | 0.9955 |
| (GAAA) | 80.9 | 0.24 | 0.08 | 0.11 | 2.03 | 1.30 | | | |
| Two Site | | | | | | | | | |
| A7U | 665.40 ± | | | | | | 2.10 ± | 1.30 ± | 0.99384 |
| (GAAA) | 30.1 | | | | | | 0.20 | 0.06 | |
| Hill | | | | | | | | | |
| A7U | 553.3 ± | 0.58 ± | 0.41 ± | −0.22 ± | 1.42 ± | −0.56 ± | | | 0.9987 |
| (AGUA) | 53.8 | 0.54 | 0.06 | 0.09 | 1.50 | 0.53 | | | |
| Two Site | | | | | | | | | |
| A7U | 501.84 ± | | | | | | 0.73 ± | 1.73 ± | 0.99685 |
| (AGUA) | 8.45 | | | | | | 0.02 | 0.07 | |
| Hill | | | | | | | | | |

Nonlinear least squares filling parameters for Mg²⁺ sensitivity for three 266 hRHO EhhRzs to Equations 1 (Double Boltzmann) and 2 (Hill) in the text. Vₘₐₓ is in units of min⁻¹. [Mg²⁺], parameters are in units of mM. Frac, k₁, k₂, and n are unitless. For the Double Boltzmann fit [Mg]_{0.5,1} and [Mg]_{0.5,2} are the Mg²⁺ concentrations at the half maximum titration points for the separate components. For the Hill fits [Mg]_{0.5} is the half-maximal titration fit. For the Double Boltzmann fitting frac is the Fraction of the overall Vₘₐₓ that is due to the first (most sensitive) component of the Mg²⁺ titration (1-frac is the fraction of Vₘₐₓ due to the less sensitive titration). For the Double Boltzmann fits k₁ and k₂ are the sensitivity (slope) parameters for the two components of the titration. For the Hill fits n is the Hill exponent parameter. AdjR² is the adjusted R² or goodness of fit. Variances obtained from fitting are standard errors of the mean. In each case the substrate was the 15-mer at a concentration of 1 μM and the EhhRz was at concentration of 100 nM.

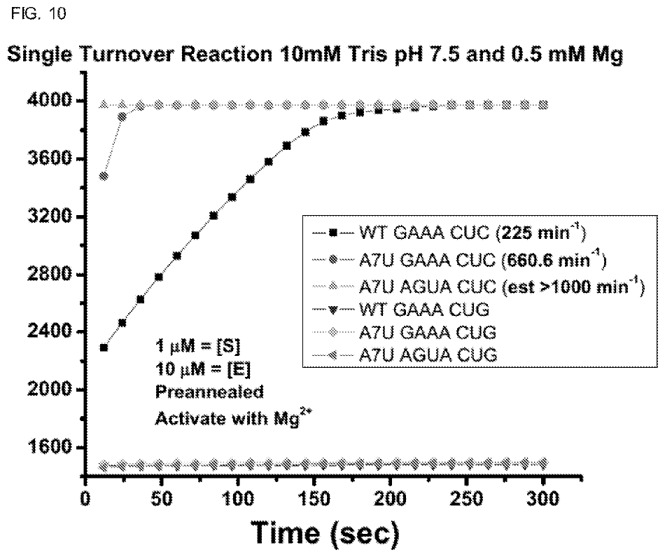

Single-turnover reaction conditions. The data presented above were collected under multi-turnover (steady state) conditions, with the substrate in excess and the reaction catalyzed by mixing the EhhRz (with Mg²⁺) with the substrate. The rate of reaction was measured at pH 7.5 with the enzyme in 10-fold molar excess over a substrate level (1 μM) that produces optical signals, with substrate and enzyme pre-annealed and with the reaction catalyzed by the addition of Mg²⁺. Under these “single-turnover” conditions, the entire population of substrate molecules is expected to be bound in Watson-Crick complex with the EhhRzs, with the cleavage reaction ready to be initiated by the Mg²⁺ cofactor (FIG. 10). At pH 7.5 with 0.5 mM Mg²⁺ and a 37° C. reaction temperature, the initial rate of reaction for the WT(GAAA) enzyme was 225 min⁻¹ for the 15-mer substrate with a cleavable CUC↓ motif. For the A7U(GAAA) enzyme, a few points were measurable, showing a rate of >660 min⁻¹. The reaction was already saturated at maximum signal by the first optical measurement for the A7U(AGUA) enzyme, showing a cleavage rate greater than 1000 min⁻¹. There was no evidence of cleavage of the non-cleavable 15-mer 266 substrate (CUG) by the three EhhRzs. For the three enzymes, the large step in fluorescence before the first optical measure is an index of very rapid cleavage processes that are not captured at this kinetic bandwidth; a rapid stopped-flow kinetics analysis is necessary.

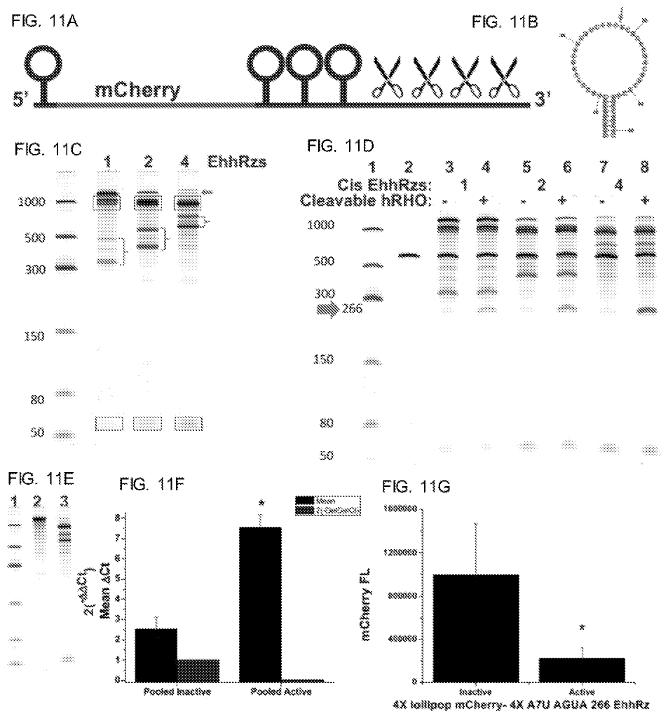

Cleavage of Large Structured Targets and Intracellular Cleavage by EhhRzs. In order to determine if the EhhRz was cleaving target within the intracellular environment, an experimental paradigm that exploits a fusion RNA between the target and a reporter RNA was utilized. Here, four 51 nt hRHO RNA target elements (“lollypops”) (240-290 nt, from NCBI Reference Sequence: NM_000539.3 were embedded, FIG. 11A) surrounding the 266 CUC cleavage site into the a cDNA for mCherry2 fluorescent protein; one target element was inserted into the 5′ UTR and three into the 3′ UTR. 2D RNA folding (MFold and RNA Structure) predicts a stem loop structure (ΔG=−12.80 kCal/mol) with a 9 bp GC-rich (66%) stem and a large single-stranded loop (33 nt, 249-281), in which the CUC cleavage site is centered with an accessible antisense binding platform (FIG. 11B). The 266 CUC element within full length hRHO mRNA was previously targeted successfully in HEK293S cells using 266 mhhRzs embedded within structured RNA scaffolds. The immediate rationale was to determine if the optimized 266 A7U AGUA EhhRz could cleave a complex target while embedded within it (in cis) or, thereafter, against an identical target presented in trans, and whether the EhhRz was capable of demonstrative cleavage in cultured human HEK293S cells. In tandem one, two or four 266 A7U AGUA EhhRzs were embedded downstream of the last target element in the 3′UTR of the mCherry fusion mRNA. Each EhhRz should have capacity to cleave at any of the four 266 lollypop target elements in the mCherry fusion mRNA. In vitro transcription of the fusion mRNA demonstrated cleavage products in denaturing gels with increasing complexity as the numbers of EhhRzs increased in the downstream region of the target fusion mRNA (FIG. 11C). The changing cleavage pattern likely indicates preferential cleavage targets with different numbers of EhhRzs present over the 3 hr and 45 min reaction (semi-combinatorial process). The three EhhRz containing RNAs generated similar large molecular weight products (3 products for 1×, 4 products for 2× and 4× EhhRzs) in different relative quantities, as well as different amounts and sizes of medium sized molecular weight products (these are liberated Cis-EhhRzs) and small sized products. Mutation of the catalytic core (G8C) in each of the EhhRzs within the 4× EhhRz fusion mRNA construct obviated the cleavage products as expected and demonstrated the true full length fusion mRNA size (doublet around 1200 nt) (green arrow).

In vitro co-synthesis transcription of the fusion mRNA and an independent 532 nt hRHO target RNA (1-532 nt of full length hRHO mRNA containing a single native 266 CUC↓ cleavage site) revealed that increasing numbers of EhhRzs in the fusion mRNA generate increasing amounts of cleavage product (size 266 nt, green arrow) (FIG. 11D). In contrast a “hardened” 532 nt target (target site CUC converted to non-cleaving CUG) abolished the 266 cleavage product. This provides evidence that Cis EhhRzs, liberated from the fusion mRNA during the reaction, are able to attack an independent structured mRNA fragment target in trans containing the wild type 266 CUC site.

CMV plasmids expressing the mCherry fusion mRNA containing 4× active 266A7U. AGUA EhhRzs or the mutated catalytically-inactivated forms of those EhhRzs (controls) were transfected into HEK293S cells. These constructs were first tested in vitro and showed that the catalytically active forms of the EhhRz promoted combinatorial cleavage of the target but the catalytically inactive forms (4 EhhRzs mutated at G8C) showed no evidence of fusion RNA target cleavage (FIG. 11E). The fusion mRNA was quantified using real-time RT/PCR, referenced to b-actin as a housekeeping mRNA, and analyzed by the 2^{-ΔΔC}^{ₜ} method using two sets of primers established for mCherry (FIG. 6F). ANOVA based comparison of the means of the active and inactive EhhRz showed that the means of the ΔCt values (mCherry—Actin) for the two primer sets were not significantly different for both the active (p=0.33189) and inactive (p=0.59287) EhhRz constructs; these values were subsequently pooled; however, prior to pooling, the individual differences for the two primer sets (mCherry—Actin) showed statistically significant differences (Set 1: ANOVA, p=1.11E-16; Bonferroni, p=9.757E-17; Set 2: ANOVA: p0.05; Bonferroni, 3.261E-24). Statistical comparison (ANOVA, post-hoc comparisons) showed a strong increase in the pooled Mean ΔCt value for the active EhhRz construct (ANOVA, p=0.05; Bonferroni, p=1.202E-38). The 2^{-ΔΔC}^{ₜ} analysis showed a nearly two log relative suppression of the mCherry mRNA with the active EhhRz (0.03146) attacking the 266 CUC sites relative to the inactive (G8C) 266 EhhRzs. The mCherry protein fluorescence was measured on a quantitative microscopic platform (Keyance) (FIG. 11G). The construct with 4 active EhhRzs (N=2633) suppressed 78% of the mCherry fluorescent protein signal relative to the mutated EhhRz plasmid (N=3348) in HEK293S cells 48 hrs post transfection (2 Sample t-test without assumption of equal variance, t=93.93, p 0.05). This clearly demonstrates that the EhhRz cleavage functionality is active in the human cultured cellular environment under conditions of cellular physiological free Mg²⁺ levels, ionic strength, viscosity, and temperature.

Discussion The hhRz is a site-specific RNA endonuclease. hhRzs (Heptazyme™ targeting the 5′ end of hepatitis C virus RNA, Herzyme™ for HER2 overexpression in breast and ovarian cancers, and Angiozyme® targeting VEGF receptor-1 for stage IV metastatic breast cancer) have been developed, systemically delivered and tested in clinical trials but failed to progress to clinical approval. The development and delivery of posttranscriptional gene silencing agents has been difficult. To be successful, an injected nuclease-resistant hhRz must be distributed pharmacodynamically in serum and enter the appropriate cells in target tissues at concentrations sufficient to ensure annealing with structured target mRNA at an accessible site to exert cleavage and therapeutic knockdown. Such a multivariate problem severely constrains the probability of success with systemic delivery.

Vector-mediated gene therapy bypasses the problems associated with systemic delivery by expressing the construct in target cells. Many studies have demonstrated a range of target knockdown efficacies for hhRzs at the mRNA and protein level via gene therapy methods. With this approach, the hhRz is overexpressed relative to the target mRNA to maximize the probability of interactions at intracellular physiological Mg²⁺ levels. However, mhhRzs have a low turnover rate of <1-2 min⁻¹ for small unstructured targets, which is generally orders of magnitude lower for structured full-sized mRNA targets. Notably, several computational and experimental approaches can identify rare regions of the folded native-full length mRNA that are accessible to these therapeutic agents in vitro or in cellulo. Improvements in hhRz kinetics, the prime variable limiting therapeutic potency, could substantially revitalize their therapeutic potential. However, when the substrate concentration exceeds that of the ribozyme (Michaelis-Menten conditions)—conditions important for therapeutics—the kinetics of the reaction are expected to be slower because of the lower collision frequency. Hence, substantially improved kinetics could maximize target knockdown at lower expression levels or delivery levels, and thereby lower the coincident risk for toxicity.

mhhRzs lack the TAEs found in naturally occurring “extended” hhRzs (xhhRzs) that are responsible for enhanced kinetic performance at cellular levels of free Mg²⁺. Subsequent structural analyses of xhhRz RNAs with upstream TAEs explained extensive biochemistry data that crystal structures of the mhhRz were unable to explain. Stem-loop II interactions with in cis TAEs are not conserved and exhibit variability in nature. Although mhhRzs have a lower probability of sampling the active state structure(s), their reaction mechanisms and catalytically active structures are similar to those of xhhRzs. Thus, there may be many structural paths (dynamic and topological) and interactions that lead to a functional catalytically active state(s). The free energy, topology, and dynamics of a more remote loop impact the probability of a successful catalytic event, because loop interactions initiate structural/dynamic transitions that propagate into the enzymatic core to affect chemical mechanistic processes (e.g., sn2 alignment, general base, reducing the charge on the phosphate during attack, general acid). Regardless, studies of xhhRzs have shown that this small catalytic RNA has greater kinetic capacity than initially estimated.

To enhance the therapeutic potential of hhRzs, their capacity to cleave targets in trans has been investigated. Cleavage rates of xhhRzs are generally improved versus mhhRzs under physiological or supraphysiological Mg²⁺ levels. However, to obtain a bulge loop structure in the upstream region to simulate an xhhRz functioning in cis, the stem I antisense flank of the hhRz in trans must be extended. From a therapeutic perspective, this would lower specificity (because an improperly bound target is not displaced and could still cleave) and decrease turnover (lower leaving rate of the upstream flank). Furthermore, extension of stem II to generate additional nonterminal loop structures will inhibit the activity of the hhRz in trans. In summary, it is difficult to transition the enzymatic properties in the naturally occurring in cis functionality (cleavage of a target after intramolecular recognition and ligation in the rolling circle replication model) to in trans functionality (therapeutic intermolecular attack). However, it has been shown that a highly active in trans mhhRz functionality (estimated at 61 min⁻¹ at physiological pH in 10 mM Mg²⁺) is accomplished by including an unpaired U7 residue on the downstream substrate flank.

The loop U7 residue in the substrate component was originally identified in many xhhRzs, in which a specific Hoogsteen base face interaction was noted with the terminal A residue (e.g., LA4) in the GUGA (GRNA) stem II loop. The Hoogsteen LA4:U7 interaction was identified in two full-length hhRz crystal structures of the xhhRzs of Schistosoma mansoni (PDB 3ZP8, class I xhhRz) and the satellite RNA of tobacco ringspot virus (TRSV) (PDB 2QUS, class III xhhRz). The interaction between the upstream antisense flank region and the stem II loop is important for meaningful cleavage at cellular levels of free Mg²⁺ (<1 mM), which is approximately an order of magnitude lower than the standard conditions used to evaluate hhRz kinetics (10 mM). Single-turnover cleavage rates of xhhRzs with upstream components can be 100-fold better than those of mhhRzs. An intramolecular (in cis) cleavage rate of 1.2 min⁻¹ was measured for the TRSV xhhRz at 0.1 mM Mg²⁺, pH 7, and 37° C.; nine other xhhRzs have rates from <0.01 to 1.40 min⁻¹. Site-specific mutagenesis revealed an interaction between the GNRA (GUGA) tetraloop capping stem II and the heptaloop (UGUGCUU) capping stem I; three of the four residues of the tetraloop were important to cleavage activity (L2.1, L2.3. and L2.4; not L2.2 [U]) and five of the seven nucleotides in the heptaloop were important (L1.1-L1.4 and L1.6); L1.1 is equivalent to the U7 substrate residue in stem I. Several cis and trans Watson-Crick and sugar:sugar interactions between the nucleotides in the stem II and stem I loops of TRSV xhhRz (PDB 1P66) were structurally modeled. Gel-based cleavage assays were used and a rate of 0.77 min⁻¹ was measured for their mhhRz (HH_{min-AL4:U1.7}) at pH 5.6 (to slow activity) and 27° C. with the mhhRz pre-annealed in 50-fold molar excess relative to its 21-mer substrate (5′ labeled with Cy3) in a reaction initiated by 10 mM Mg²⁺; rates extrapolated to pH 7.5 were 61±9 min⁻¹ with the assumption that pH affects the cleavage rate (k) and not the product leaving rate. An additional construct extended the extrapolated single-turnover cleavage rate to 95±20 min⁻¹ at 10 mM Mg²⁺. Control reactions in which the upstream antisense flank was extended and a U7 residue was included in a Watson-Crick base pair had lower rates than those with mhhRzs.

Single-turnover conditions in conventional gel-based cleavage assays reflect the cleavage rate of the hhRz (k₂). The optical turnover methods disclosed herein requires both the cleavage rate and at least one product dissociation rate (to separate the FAM on the upstream cleavage product from the BHQ1 on the downstream product). Under these conditions (0.25 mM Mg²⁺), the maximum scaled turnover activity was 69.70 T 9.27 nM min⁻¹ for the best EhhRz (A7U AGUA), which is 50-fold better than their maximum single-turnover rate of 1.4 min⁻¹ at 0.1 mM Mg²⁺. With 10 mM Mg²⁺, simulating the conditions used by others, the most active enzyme [A7U(AGUA)] had a scaled turnover rate of 486.51±43.47 nM min⁻¹, which is 5.1-fold better than their maximum (estimated) single-turnover activity of 95 min⁻¹ assessed at 10 mM Mg²⁺. The single-turnover rates at physiological Mg²⁺ (0.5 mM) were greater than 680 nM min⁻¹ for the A7U(GAAA) EhhRz (7.16-fold) and were likely greater than 1,000 nM min⁻¹ (>10.5-fold) for the best construct [A7U(AGUA)] relative to the best construct by others measured under single-turnover conditions with 10 mM Mg²⁺ The measured turnover rate for HH16 (˜9 nM min⁻¹) in this optical assay at 0.5 mM Mg²⁺ and 37° C. (pH 7.5) is greater than the 1-2 min⁻¹ measured under single-turnover conditions in various studies under “standard” conditions (25° C., 10 mM Mg²⁺, and pH 7.5) in gel based assays; this can be attributed, at least in part, to the higher reaction temperature. Relative to the classic HH16, under the same substrate excess (turnover) conditions at 37° C. and 0.5 mM Mg²⁺ the improvement is 18.8-fold for the A7U(AGUA) enzyme with the assay described herein. The best EhhRz has the same enzyme core and the same stem II sequence as HH16; the primary differences are between the stem TT tetraloop and the antisense flanks.

Enhanced hhRz activity has been reported. The gel-based assays supporting these observations are generally conducted under non-Michaelis-Menten conditions of enzyme excess [pre-annealed [E]>[S]) and nonphysiological “standard” Mg²⁺ conditions (10 mM Mg²⁺). For instance, a class I/II mhhRz (HH1α) with single-turnover activity of 10 min⁻¹ was identified under standard conditions. By grafting elements of HH1α onto HH16 (0.4 min⁻¹) and decreasing the pH to 6.5 to slow kinetics, it was determined that the first two base pairs (U:A/A:U) of the stem I antisense flank were responsible for enhanced activity and that a stem II length>4 bp was inhibitory. Very high cleavage rates (˜870 min⁻¹, 25° C.) have also been reported from an in trans form of the Schistosoma xhhRz tested under single-turnover (enzyme excess) conditions with very high levels of Mg²⁺ (200 mM; K_{d}, ˜40 mM) and high pH (8.5), which, though far from physiological, demonstrated the catalytic potential of RNA enzymes; U6 and U7 residues were present in the substrate strand, and the stem I antisense flank had a total of 12 nt. Evolutionary techniques have also been used to identify an in trans hhRz (“RzB”) which simulated the structure and function of the peach latent mosaic viroid xhhRz operating in cis. RzB uses a long stem I antisense flank (11 nt) with a central UAA bulge that interacts with a 6-nt stem II loop (5′-UGGGAU-3′, similar to the naturally occurring sequence of peach latent mosaic viroid hhRz [UGAGAU]). RzB demonstrated rates of 1.8 min⁻¹ at 37° C. with 0.5 mM Mg²⁺ under single-turnover pre-annealed reaction conditions with enzyme in 10-fold molar excess over substrate, which did not harbor a U7 residue. Pre-steady state reaction conditions were investigated with the in trans RzB and measured “extraordinary” cleavage rates (780 min⁻¹ at pH 7.4, 37° C., and 1 mM Mg²⁺) in a stopped-flow apparatus with substrate and enzyme pre-annealed in complex (10:1 [E]/[S]). Reactions occurred with multiphasic kinetics, and the fastest single-turnover rate component represented ˜5% of the overall cleavage extent, which otherwise progressed to 80% cleavage. This study established the upper limit of the hhRz cleavage kinetics at the time and suggested means to achieve such efficiencies. The goal was to utilize RzB for intracellular target knockdown applications, and it was initially tested in vitro against a strongly structured fragment of HIV RNA, for which cleavage was orders of magnitude slower. A translational study with the RzB format, embedded internally within a modified VA1 scaffold RNA was performed, but it did not improve target knockdown relative to that with a mhhRz without TAEs targeting the same accessible site.

Evaluating EhhRz Cleavage Capacity against Structured mRNA targets. In order to further validate the EhhRz findings, hRHO 266 A7U AGUA, the lead agent, was tested against much larger structured mRNA targets that are realistic to the therapeutic challenge. Structured hRHO 266 RNA targets (lollypops) were embedded into the 5′UTR (1 copy) and 3′UTR (3 copies) of mCherry2 mRNA to create “fusion mRNA” that presents the folded 266 EhhRz targets (51 nt stem loops) in a readily assayed reporter mRNA that is expected to translate a stable monomeric red fluorescence protein that is readily assayed quantitatively (4×-lollypop-mCherry2) (FIG. 11A). The lollypop hRHO element containing the 266 CUC site (FIG. 6B) is predicted to be a stable structure with a large single stranded loop which is expected to allow efficient targeting. One, two or four A7U AGUA 266 hhRzs were appended downstream of the last hRHO 266 lollypop in the 3′UTR of the fusion mRNA. Cleavage by the embedded EhhRzs within the 3′UTR is expected to alter the half-life of the fusion mRNA and reduce its steady state level upon which the translational apparatus operates, whereas cleavage in the 5′UTR lollypop should abrogate protein synthesis. It was found that EhhRzs embedded downstream of the 266 lollypops in the 3′UTR were able to cleave in cis at various lollypop sites in the fusion mRNA concurrent with transcription in vitro. The banding pattern was consistent with combinatorial cleavage and where the extent of cleavage was proportional to the number of EhhRzs in the fusion mRNA (FIG. 6C). This provides evidence that the EhhRz can bind to and cleave targets in cis or in trans after being liberated by self-cleavage out of the fusion mRNA transcript. Mutation of the enzyme core of the EhhRzs embedded obviates the banding pattern demonstrating that this results explicitly from EhhRz cleavage at various sites in the fusion mRNA. To test for in trans functional capacity co-synthesis transcription of a fragment of the hRHO mRNA (1-532 nt) which presents a monomeric hRHO band and the fusion mRNA containing the EhhRzs was performed. The 532 nt hRHO fragment RNA contains a single 266 CUC cleavage site and the appearance of a 266 product band in proportion to the amount of EhhRzs embedded. This evidence underscores the capacity to cleave an independent target in trans, which is an important element for a gene based therapeutic (FIG. 6D). Conversion of the single CUCd cleavage motif in the hRHO 532 fragment target to a CUG, which does not cleave, obviates the cleavage banding, and confirms that the 266 product results from cleavage in trans of the hRHO mRNA by the liberated EhhRz elements (FIG. 6E). Transfection of the 4×-lollypop-mCherry2 266 A7U AGUA EhhRz (4×) expression construct into cultured HEK293S cells promotes marked knockdown (>1 log order) of mCherry2 mRNA measured by real time RT/PCR and the 2^{-ΔΔCt} method relative to a control construct in which all 4 EhhRzs were mutated (G8C) to catalytically inactivated enzymes (FIG. 6F). mCherry2 protein fluorescence also showed marked and significant knockdown (˜80%), relative to the construct which mutates the EhhRzs to a catalytically inactive state (G8C) (FIG. 6G). These results demonstrate that EhhRzs function within the intracellular environment to cleave NUH↓ sites within large structured targets where physiological conditions of free Mg²⁺, temperature, ionic strength, and viscosity exist.

Practical implications for clinical or biotechnological translation. The data described herein clearly shows that the EhhRz A7U(AGUA) achieves scaled turnover rates of 314 nM min⁻¹ under steady-state (substrate excess) conditions at cellular levels of free Mg²⁺ (≤1 mM) and physiological temperature in vitro. This EhhRz achieves saturation cleavage of 1 μM substrate within 5 min, as expected for this level of turnover at 0.5 mM Mg² (FIG. 1B). EhhRz is designed to cleave an accessible region in RHO mRNA, a target for knockdown/reconstitute therapy in autosomal dominant retinitis pigmentosa and knockdown therapy in age-related macular degeneration. The efficiency of the fastest enzyme to date [A7U(AGUA)](Vₘₐₓ=214.62±27.92 min⁻¹, Kₘ=1344.08±211.13 nM) is 1.60×10⁸ M⁻¹ min⁻¹, which is over I log-fold more efficient than the 525 RHO mhhRz, which had a K, of 152 nM and k_{cat} of 0.78 min⁻¹ under substrate-excess conditions at 2 mM Mg²⁺ and 37° C., or an efficiency of 5.13×10⁶ M⁻¹ mini. The Kₘ of the EhhRz tested here is on the same scale, but the k_{obs} at lower levels of Mg²⁺ (0.5 mM) is over two orders of magnitude faster, making for an enzymatic efficiency that rivals that of RNase A for 266 A7U(AGUA). The Kₘ of the enzyme is largely related to the antisense flank energy required for molecular recognition (binding) of the target sequence and will predictably vary with antisense flank length and composition, which can be optimized to maximize selectivity and product leaving rates. The k_{obs} is a major chemical property of the enzyme proper, for which EhhRzs clearly demonstrate log-order improvement over hhRzs or xhhRzs. When evaluating EhhRzs under single-turnover conditions (10-fold molar excess of enzyme over substrate [at 1 μM]), pre-annealed and initiated by 0.5 mM Mg²⁺, the rate was unable to be measured with current optical methods of the A7U(AGUA) enzyme because of the rapid speed (estimated >1000 nM min⁻¹), the A7U(GAAA) enzyme was estimated to be at >680 nM min⁻¹ and a rate of approximately 273 nM min⁻¹ was obtained for the WT(GAAA) enzyme. Reduced pH was not explored because the optical assay designed and described herein depends upon product release, and base pairing can be affected by pH. Rapid kinetic assays (stopped-flow) are needed to investigate these rates under physiological conditions.

EhhRz 266 A7U(AGUA) achieves rates well beyond the expected performance of an mhhRz. This finding is likely because EhhRz uses more of the available mechanisms for rate enhancement than just the a (sn2-orientational alignment of the 2-OH, the scissile bond, and the proton donor) and 7 (general base mechanism to abstract a proton from the 2-OH, thought to relate to properties of GI 2) processes used by the mhhRz. The additional use of the R (decreasing the charge accumulation on the phosphate of the activated state) and 8 (efficient protonation of the leaving group by the general acid, thought to relate to properties of G8) mechanisms could bring the catalytic efficiency of the EhhRz to the scale of RNase A, which is thought to use the physical mechanisms for phosphodiester cleavage.

The GNRA tetraloops consistently supported high turnover activity for the model EhhRz, whereas the single model UUCG and CUUG tetraloops poorly supported high turnover activity. The first and last nucleotides of these canonical tetraloops form base pairs, and these tetraloops have varied stability. These tetraloops also have varied structures, which do not shed light on the enhanced activity of the EhhRzs studied here. The distinct empirical finding is the A7U(AGUA) stem loop II cap, which uses a less-well-studied AGNN tetraloop that does not have an L1:L4 base pair. Rather, the first and second nucleotides stack on the underlying 5′ nucleotide of the closing base pair of the underlying stem (stem II in hhRz) and the second G residue is in a syn configuration, which causes outpocketing of the phosphodiester bond with the third nonconserved N residue (Lebars et al., 2001). It is the A7U(AGUA) EhhRz construct that demonstrates the most rapid kinetics and a clear cooperative transition in the presence of Mg²⁺. The roles of different tetraloops have evolved for interactions with other RNAs or proteins. One may speculate that the structural and thermodynamic properties of the AGUA tetraloop (and, to a similar extent, the AUUA, AAUA, and AAAA tetraloops; see FIGS. 6A, 6B) facilitate the Mg²⁺-dependent conformational changes that drive and stabilize a highly efficient catalytically active state that maximizes the engagement of the four potential contributors (a, y, p, and 6) to accelerate phosphodiester cleavage.

There are several consequences of the observations described here. First, an EhhRz with such substantial improvements in enzymatic performance under physiological conditions requires lower levels of expression in/delivery to target cells to achieve therapeutic target mRNA knockdown, thus reducing the toxicity from RNA-sensing pathways (e.g., RIG, PKR) or off-target effects, which are concentration dependent. The mhhRz that showed some success in rescue of a mouse model of photoreceptor degeneration due to mutant RHO was also trialed in a dog model with mutant RHO, but had to be delivered at such high levels in a vector that it was toxic. EhhRzs thus can revitalize the utility of catalytic nucleic acid therapeutics. The results described herein show that EhhRzs have definitive capacity for cleavage of specific target sites within the intracellular environment. It is contemplated that these agents can be expressed or delivered in the in trans (intermolecular) context. Second, the broad range of rates from a limited variety (<10%) of stem 11 tetraloop cap mutants demonstrates that the structural and functional mechanistic biology of EhhRzs may have further catalytic potential. It also shows that RNA catalytics with native nucleotide chemistries (G, U, C, and A) have the capacity for turnover rates that are much better than the mhhRz format at 1-2 min⁻¹, which utilizes two of four rate acceleration mechanisms. Third, given that the stem II loop variants had identical antisense flanks and that the reaction and buffer conditions were otherwise identical, the product leaving rates should also be identical. As a result, the cleavage rate, k₂, cannot be a constant. Fourth, because the stem II capping loop can be functionally varied to enhance turnover rates with variable stem I interactions, the loop sequence could be improved for particular upstream flanks (receptors) in arbitrary targets (consider GNRA tetraloop receptors). Fifth, the small size of the EhhRz can be improved with non-natural nucleotides at specific positions for further rate enhancement. The enzyme efficiency (k_{cat}/Kₘ), which already rivals that of RNase A, might be improved further if the k_{cat} can be improved by nucleotide chemistries tuned to enhance the known mechanism of reaction (e.g., designing the pKₐs of G12 and G8 residues to bring them closer to neutrality, as in RNase A, which uses two histidine residues [general base H12 and general acid H119] in the active site). Sixth, recently, it was shown that synthetic antisense oligodeoxynucleotides injected into the vitreous of the eye (a closed volume space) can be used to treat mutant pre-mRNA splicing defects in photoreceptor nuclei; this study speaks volumes to a new emerging class of nucleic acid therapeutics for outer or inner retinal degenerative conditions, or perhaps ocular conditions in general. Seventh, based on a prior preclinical study an ongoing human clinical trial (NCT04123626; clinicaltrials.gov; ProQR Therapeutics, QR-1123) is evaluating use of next generation antisense agents, injected into the human vitreous body, in order to suppress the mutant P23H RHO mRNA of patients with an autosomal dominant form of retinitis pigmentosa, an inherited retinal dystrophy; use of EhhRzs in this context can provide an improvement to antisense agents which have slower kinetic turnover and must usurp host cell machinery (e.g., RNaseH) for cleavage of target mRNAs. The properties of EhhRzs described herein further underscore their use in human nucleic acid therapeutics (gene therapy or synthetic nucleic acids) or in a biotechnological capacity (e.g., aptazymes); given the range of turnover rates with different Stem-II tetraloops EhhRzs could be “tuned” for function in specific applications.

Material and methods. Reagents. Synthetic DNA templates, primers, and RNA oligonucleotides were synthesized by IDT Technologies (Coralville, IA). RNA oligonucleotides were subjected to analytical reverse-phase high-performance liquid chromatography (HPLC) and purified by RNase-free HPLC. The lyophilized material supplied was solubilized in RNase-free double deionized water to achieve stock concentrations of 100 M.

MEGAshortscript (AM1354) and MEGAscript (AM 1333) in vitro transcription kits, and Lipofectamine 3000 (L3000008) were obtained from ThermoFisher Scientific (Waltham, MA).

RNA Clean & Concentrator kit (Zymo Research, Irvine, CA, catalog number R1017). Restriction enzymes and T4 DNA ligase (NEB, Ipswitch, MA).

mCherry2-C1 was obtained from Addgene (Addgene plasmid #54563; //n2t.net/addgene:54563; RRID:Addgene_54563).

In vitro transcription. In vitro transcription templates were generated by PCR using PfuUltra II. PCR reactions were carried out with 100 ng of single-stranded DNA template, 0.5 μM upstream primer, 0.5 μM downstream primer, and 50 μl of PfuUltra II Hotstart 2× master mix (catalog number 600850; Agilent, Santa Clara, CA) in a total volume of 100 μl according to the manufacturers' recommendations. The following constant single-stranded DNA sequence includes the T7 promoter (underlined) and an upstream leader sequence:

| (SEQ ID NO: 49) | |
|---|---|
| | 5′-CCATGATTACGCCAAGCTTAATACGACTCACTATAG⁺¹GG-3′. |

Templates generated from the T7 promoter incorporated two G's following the G⁺¹ for highly efficient transcription (Khvorova et al., 2003; Conrad et al., 2020). The constant region was extended by the following single-stranded sequences: WT 266, 5′-TAGAGCGTCTGATGAGGCCGAAAGGCCGAAAGGAAGTT-3′ (SEQ ID NO: 50); A7U 266, 5′-TTGAGCGTCTGATGAGGCCGAAAGGCCGAAAGGAAGTT-3′ (SEQ ID NO: 51); A7U 266 G8C, 5′-TTGAGCGTCTGATCAGGCCGAAAGGCCGAAAGGAAGTT-3′ (SEQ ID NO: 52); A7G, 5′-TGGAGCGTCTGATGAGGCCGAAAGGCCGAAAGGAAGTT-3′ (SEQ ID NO: 53); and A7U tetraloop cap variations, 5′-TTGAGCGTCTGATGAGGCC(N⁴)GGCCGAAAGGAAGTT-3′ (SEQ ID NO: 54); the “A7” residue and the stem II tetraloop sequences are underlined. N⁴ is not a randomized sequence but refers to the sense sequence of the various tetraloop sequences tested in this study (see FIGS. 6A, B). The constant PCR sense primer had the following sequence: 5′-CCATGATTACGCCAAGCTTAATACG-3′ (SEQ ID NO: 55). The PCR antisense primer, 5′-AACTTCCTTTCGGCCTTTCGGC-3′ (SEQ ID NO: 56) was used to generate WT 266 and A7U 266 PCR products. The antisense primer for tetraloop variations was 5′-AACTTCCTTTCGGCC(N⁴)GGC-3′ (SEQ ID NO: 57). The constant single-stranded DNA sequence for hhRz 16 includes the T7 promoter (underlined) and an upstream leader sequence:

| (SEQ ID NO: 58) | |
|---|---|
| | 5′-CCATGATTACGCCAAGCTTAATACGACTCACTATAG⁺¹. |

The constant region was extended by the following single-stranded sequences: WT hhRz 16, 5′-GCGATGACCTGATGAGGCCGAAAGGCCGAAACGTTCCC-3′ (SEQ ID NO: 59); hhRz 16 G8C, 5′-GCGATGACCTGATCAGGCCGAAAGGCCGAAACGTTCCC-3′ (SEQ ID NO: 60). The anti-sense primer for hhRz 16, 5′-GGGAACGTTTCGGCCTTTCGG-3′ (SEQ ID NO: 61). PCR templates were purified using a QIAquick PCR purification kit (Qiagen, Hilden, Germany) and quantified by optical density at 260 nm (OD₂₆0) on a Nanodrop 2000 microfluidic UV-visible absorption spectrophotometer (ThermoFisher Scientific, Waltham, MA).

The 20-1 in vitro transcription reaction contained 500 ng of PCR product, 7.5 mM of each ribonucleotide triphosphate, 2 μl T7 RNA polymerase and the assay was incubated at 37° C. for 3 h 45 min (MEGAshort script, ThermoFisher). Plasmid DNA was digested with 2U of Turbo DNase (RNase-free; Invitrogen) for 15 min at 37° C. The RNA was cleaned using a Zymo RNA Clean & Concentrator-25 (catalog number R1017; Zymo Research, Irvine CA) according to the manufacturers' recommendations and resuspended in RNase-free, DNase-free deionized water. The RNA concentration was quantified and quality controlled via the OD₂₆₀ and OD₂₈₀ measured on a Nanodrop 2000 spectrophotometer. hhRz RNAs were stored at −80° C. until use, thawed at room temperature, and then maintained at 4° C. until use.

In vitro transcription for larger templates (e.g., mCherry fusion-target mRNA, hRHO 532 target) were conducted in the same manner as above except that a kit optimized for longer transcripts was used (MEGAscript, ThermoFisher, Waltham MA).

In Vitro Co-Synthesis Cleavage Assays. PCR generated templates were prepared for in vitro transcription of the CIS mCherry fusion-target mRNA with 1, 2, 4 active and G8C (catalytic inactive) hhRzs.

The sense primer including the T7 promoter
(underlined)
(SEQ ID NO: 62)
5′-CCATGATTACGCCAAGCTTAATACGACTCACTATAGGGTTTAGTGAA
CCGTCAGATCCGC-3′
and
the anti-sense primer
(SEQ ID NO: 63)
5′-AGAATGCAGTGAAAAAAATGCTTTATTTGTGAAATTTGTG-3′.

PCR generated template for in vitro transcription of hRHO 532.

The sense primer including the T7 promoter
(underlined)
(SEQ ID NO: 64)
5′-CCATGATTACGCCAAGCTTAATACGACTCACTATAGGGAGAGTCATC
CAGCTGGAGCCCT-3′
and
the anti-sense primer
(SEQ ID NO: 65)
5′-AAGTTGCTCATGGGCTTACACACCAC-3′.

In vitro transcription of larger transcripts was conducted using the MEGAscript kit optimized for longer transcripts with the amount of PCR template varying from MEGAshortscript reactions. For the in vitro transcription of the CIS mCherry-reporter fusion-target mRNA with 1,2,4 active or inactive (G8C) hhRzs, 100 ng of PCR template was used and, upon completion of the reaction, 25 nM of this product was run on a 4% PAGE UREA gel, and visualized with SYBR Gold (ThermoFisher Scientific, catalogue number S11494) on a ChemiDoc MP system (Bio-Rad, Hercules, CA). The co-synthesis in vitro transcription reaction was performed with 9 nM of the PCR product for the CIS mCherry-reporter fusion-target mRNA with 1, 2, or 4 hhRzs and 3 nM of PCR product for the hRHO 532 with 350 ng of total RNA of the reaction run on a 4% PAGE UREA gel and visualized as stated herein.

Reporter-Plasmid Construction. The 5′ 51 nt 266 target was cloned into mCherry2-C1 by NheI and AgeI. The single stranded template sequence 5′-GGTTTAGTGAACCGTCAGATCCGCTAGC GTGCTGGGCTTCCCCATCAACTTCCTCACGCTCTACGTCACCGTCCAGCACACCG GTCGCCACCATG-3′ (SEQ ID NO: 66) was used to generate a PCR product with the sense primer 5′-GGTTTAGTGAACCGTCAGATCCGC-3′ (SEQ ID NO: 67) and anti-sense primer 5′-CATGGTGGCGACCGGTGTG-3′ (SEQ ID NO: 68) which was cut with NheI and AgeI (recognition sequences underlined). The 3′ 266 targets were cloned into mCherry2-C1 by BsrGI and BamHI. The single stranded template sequence incorporating an in-frame stop (underlined) 5′-AGTCCGGACTCAGATCTTAAGTGCTGGGCTTCCCCATCAACTTCCTCACGCTCTA CGTCACCGTCCAGCACCGAGCTCAAGCTTGTGCTGGGCTTCCCCATCAACTTCCT CACGCTCTACGTCACCGTCCAGCACCGAATTCTGCAGTGTGCTGGGCTTCCCCAT CAACTTCCTCACGCTCTACGTCACCGTCCAGCAC-3′ (SEQ ID NO: 69) was used to generate a PCR product with the sense primer 5′-GCGGCTTGTACAAGTCCGGACTCAGATCTTAAGTGCTG-3′ (SEQ ID NO: 70) and anti-sense primer 5′-GCGGCTGGATCCGTGCTGGACGGTGACGTAGAGC-3′ (SEQ ID NO: 71) which was cut with BsrGI and BamHI (recognition sequences underlined). New clones were generated using T4 DNA ligase and propagated with One Shot Stbl3 Chemically Competent E. coli (ThermoFisher Scientific, catalogue number C7373-03) to prevent recombination of repeated targets. Clones were verified by sequencing.

Reporter-Plasmid with CIS hhRz Construction. The mCherry reporter construct with embedded target elements was digested with BamHI to accept the CIS hhRz's to produce constructs with 1 and 2 hhRz's. A PCR product was generated with the single stranded template AAAAAAAAAAAAAAAGGGTTGAGCGTCTGATGAAGCCAGTAGGCCGAAAGGAA GTTAAAAAAAAAAAAAAA (SEQ ID NO: 73) with the sense primer 5′-GCGGCTGGATCCAAAAAAAAAAAAAAAGGGTTGAGCGTCTGATG-3′ (SEQ ID NO: 74) and the anti-sense primer 5′-GCGGCTGGATCCTTTTTTTTTTTTTTTAACTTCCTTTCGGCCTACTGG-3′ (SEQ ID NO: 75) and digested with BamHI (recognition sequence underlined). For incorporating four hhRz's the reporter construct was cut with BsrGI and HpaI to accept the following insert (GenScript Biotech, Piscataway, NJ) cut with BsrGI and HpaI (recognition sequence underlined) TGTACAAGTCCGGACTCAGATCTTAAGTGCTGGGCTTCCCCATCAACTTCCTCAC GCTCTACGTCACCGTCCAGCACCGAGCTCAAGCTTGTGCTGGGCTTCCCCATCAA CTTCCTCACGCTCTACGTCACCGTCCAGCACCGAATTCTGCAGTGTGCTGGGCTT CCCCATCAACTTCCTCACGCTCTACGTCACCGTCCAGCACGGATCCAAAAAAAAA AAAAAAGGGTTGAGCGTCTGATGAGGCCAGTAGGCCGAAAGGAAGTTAAAAAA AAAAAAAAGGATCCAAAAAAAAAAAAAAAAGGGTTGAGCGTCTGATGAGGCCA GTAGGCCGAAAGGAAGTTAAAAAAAAAAAAAAAGGATCCAAAAAAAAAAAAA AAAGGGTTGAGCGTCTGATGAGGCCAGTAGGCCGAAAGGAAGTTAAAAAAAAA AAAAAAGGATCCAAAAAAAAAAAAAAAGGGTTGAGCGTCTGATGAGGCCAGTA GGCCGAAAGGAAGTTAAAAAAAAAAAAAAAGGATCCACCGGATCTAGATAACT GATCATAATCAGCCATACCACATTTGTAGAGGTTTTACTTGCTTTAAAAAACCTC CCACACCTCCCCCTGAACCTGAAACATAAAATGAATGCAATTGTTGTTGTTAAC (SEQ ID NO: 76). The catalytic inactive hhRz was established by site directed mutagenesis at G8C (underlined) with the sense primer 5′-GGGTTGAGCGTCTGATCAGGCCAGTAGG-3′ (SEQ ID NO: 77) and the anti-sense primer 5′-CCTACTGGCCTGATCAGACGCTCAACCC-3′ (SEQ ID NO: 78) using PfuUltra II Hotstart polymerase and propagated in Stbl3 competent cells. Clones were sequenced for verification.

Cell Culture. Suspension-adapted human embryonic kidney cells (HEK293S) were maintained in D-MEM/F-12 media (ThermoFisher Scientific, Waltham, MA, catalogue number 12400-0240) supplemented with 10% (v/v) heat inactivated bovine serum (ThermoFisher Scientific, catalogue number 26170043), 2 mM L-glutamine, and penicillin/streptomycin. Functional assays were performed in 6 well plates (Corning Incorporated, Corning, NY, catalogue number 353046) treated with 1 mg/ml of Poly-L-lysine hydrobromide (Millipore Sigma, Burlington, MA, catalogue number P1274-100 mg, mol wt: 70,000-150,000 g/mol) for 30 minutes.

Cell Transfections and Target Protein Analysis. HEK293S cells were plated on 6 well plates to reach 50-60% confluency at the time of transfection. Cells were co-transfected per well with empty pcDNA3.1 Hygro 950 ng and 50 ng of the CIS mCherry fusion-target mRNA with 4 active or 4 G8C (catalytic inactive) hhRzs with Lipofectamine-3000 according to manufacturer's recommendations. 48 hrs post transfection the 6 well plate is placed on the stage of the Keyence microscope (Osaka, Japan, Model BZ-X810) and still images are taken in Image Cytometer mode. Under phase contrast microscopy (parameters: transmitted light 25%, aperture stop 20%, resolution sensitivity set at standard and an exposure of 1/7500s, uniform density positions (50-60) per well were selected with the 4× PlanApo λ, (0.20NA/20.00 mm) objective. Once phase positions are established the objective is placed in the middle of the well and epifluorescence is measured with a Texas Red filter cube (Ex560/40, DM585, BA 630/75), in low photobleach mode, with excitation light set at 100%, resolution sensitivity set at standard, and brightness exposure set to 1/35s). Auto focus is used to establish the imaging plane and images are collected in batch capture. Kevence BZ-X800 software is used to analyze images. The epifluorescence images are analyzed against a conditions file that provides the threshold fluorescence parameters. Once images are analyzed and saved they undergo further processing in Excel using an Expedited Kevence VBA Template to extract the average brightness integration data for each image.

Real-Time Quantitative Reverse Transcription-PCR. Total RNA was purified from transfected cell cultures 48 hours post-transfection with RNeasy plus mini kit following manufactures protocol (Qiagen, Hilden, Germany, catalogue number 74134). An additional DNase I treatment was performed on the RNA binding column at the RW1 wash buffer step (RNase-Free DNase set, Qiagen, Hilden, Germany, catalogue number 79254). cDNA synthesis was performed using 500 ng of total RNA with the AffinityScript Reverse Transcriptase system (Agilent, Santa Clara, CA, catalogue number 600559) using the supplied oligo(dT) primers. Quantitative PCR for mCherry was performed in a CFX Opus 96 RT-PCR System run with Bio-Rad CFX Maestro software (Bio-Rad, Hercules, CA). Primers and probes were designed for two regions on the mCherrry reading frame. Set 1: sense 5′-GACTACTTGAAGCTGTCCTTCC-3′ (SEQ ID NO: 79), probe 5′-/56-FAM/CAATTGGGA/ZEN/GCGCGTGATGAACTT (SEQ ID NO: 80)/3IABkFQ/-3′, and anti-sense 5′-CGCAGCTTCACCTTGTAGAT-3′ (SEQ ID NO: 81). Set 2: sense 5′-GAGATCAAGCAGAGGCTGAA-3′ (SEQ ID NO: 82), probe 5′-/56-FAM/AGACCACCT/ZEN/ACAAGGCCAAGAAGC (SEQ ID NO: 83)/3IABkFQ/-3′ (and anti-sense 5′-ACTTGATGTCGACGTTGTAGG-3′ (SEQ ID NO: 84). Human β-actin gene was used as an internal control with the following primers, sense 5′-AATTTGGAGGCTTCTTTGCCACC-3′ (SEQ ID NO: 85), probe 5′-/56-FAM/AAATTGCCCTGTGGTCCTTGGTGGT/3BHQ1/-3′ (SEQ ID NO: 86), and anti-sense 5′-AGTTGCTCATGGGCTTACACACCA-3′ (SEQ ID NO: 87). Primers were designed using PrimerQuest Tool software (Integrated DNA Technologies, Coralville, IA). Primer sets were analyzed on plasmid DNA and cDNA's to demonstrate their specificity. Quantitative PCR reactions were assembled by mixing equal volumes of PCR primers and probe to a final concentration of 0.25 μM with AmpliTaq Gold 360 Master mix (Applied Biosystems; ThermoFisher Scientific, catalogue number 4398881), and 5 μl from a 5 fold dilution of the cDNA. Samples were pipetted into hard-shell thin wall 96-well PCR plates (Bio-Rad, Hercules, CA, catalogue number HSP9601) an adhesive seal was placed on the plate and briefly spun before placed into the RT-PCR machine. Thermocycler conditions were 94° C. (4 minutes) followed by 40 cycles at 94° C. (1 minutes), 57° C. (30 seconds), and 72° C. (15 seconds, FAM detection). cDNA samples were analyzed in triplicate for mCherry and β-Actin Ct values and the 2^{-ΔΔCt} method was performed to establish relative fold levels of mRNAs (Livak and Schmittgen, 2001).

Fluorescence-based real-time ribozyme cleavage assay. Target mRNA was designed as a synthetic 15-mer human RHO mRNA substrate or a 14-mer RHO substrate containing a fluorescent dye (FAM) at the 5′ end and a quenching dye (black hole quencher 1 [BHQ-1]) at the 3′ end. The FAM is attached to the 5′ nucleotide phosphate through a 6-carbon linker (IDT). FAM has an excitation maximum of 495 nm and emission maximum 520 nm. BHQ-1 has an absorbance maximum at 534 nm; the extinction coefficient at absorbance maximum is 34,000 M⁻¹ cm⁻¹. The synthetic substrates were prepared by IDT, RNase-free HPLC purification, and quality controlled by the manufacturer. The binding, cleavage, and dissociation of products separates the upstream FAM-tagged fragment from the downstream BHQ1-tagged fragment to enable detection of FAM fluorescence, which is measured in real time with a Smart Cycler II thermal cycler (Cepheid Inc., Sunnyvale, CA) using appropriate excitation (LED) and emission filtering for FAM. The thermal cycler was programmed to sample the reaction at constant temperature (37° C.). For short reaction cycles (300 sec), the thermocycler sampled the FAM fluorescence every 12 sec (6 sec dwell time and 6 sec of read time with the measurement made at the end of each read time cycle); for longer reaction time courses, sampling occurred every 120 sec. Steady-state reactions (substrate excess) were linear in quality, and the rate of cleavage was estimated for each reaction in Excel, within the first 25 optical samples (within 300 sec), based on the point of rise of FAM fluorescence (intercept) and the approach to saturation of the photodiode in the particular reaction wells used in each experiment. Initial rate fitting was conducted by eye, extending from the initial point(s) of the reaction along the linear aspect of the fluorescence increase for the first ten points (120 sec). Steady-state (turnover) assays were conducted at a 1:10 molar ratio of hhRz to substrate and were initiated by adding the hhRz to a preformed solution containing buffer, substrate RNA, and Mg²⁺, with the components prechilled on ice. Pre-steady-state and single-turnover reactions were conducted at a 10:1 molar ratio of hhRz to substrate in 10 mM Tris-HCl buffer (pH 7.5) with pre-annealing (95° C. for 2 min, 65° C. for 2 min, and 27° C. until reaction was started); the reaction was catalyzed by adding Mg²⁺ from a concentrated stock with rapid manual mixing and pulse centrifugation in the Cepheid cuvette. Pre-steady-state (single-turnover kinetic) reactions were fit by nonlinear least-squares minimization (Marquardt analysis).

For the HH16 hhRz experiments, the native HH16 substrate (17-mer) (Hertel et al., 1994, 1996; Hertel and Uhlenbeck, 1995) was modified by adding a 5′ A residue (to add 6-carboxyfluorescein [FAM] and avoid quenching by the 5′ G present in the 17-mer). The sequence of the HH16 substrate (18-mer) was 5′-AGGGAACGUCGUCGUCGC-3′ (SEQ ID NO: 88). This substrate forms 8 nt of Watson-Crick base pairing on both flanks of the HH16 enzyme. The native 17-mer HH16 substrate had the same number of predicted base pairs (four) and structure of the 18-mer HH16 substrate used and the same minimal free energy (see FIG. 4).

Gel-based cleavage assay. An in vitro cleavage assay was initiated by adding 100 nM EhhRz to a 25-1 reaction mixture containing 1 μM of substrate, 10 mM Tris-HCl (pH 7.5), and 0.5 mM MgCl₂. The reaction was incubated at 37° C. for 5 min and then terminated by adding 25 μl of Gel Loading Buffer II (Invitrogen) and incubating at 95° C. for 5 min. Samples were run on denaturing 12% polyacrylamide 8 M urea gels at 50 mV for approximately 3 h. The gel was removed from the box and placed in 1× Tris-borate-EDTA buffer. The FAM dye moiety at the 5′ end of either the substrate (15- or 14-mer) or the upstream cleavage product (8-mer) was visualized in the gel by using a ChemiDoc MP system (Bio-Rad, Hercules, CA) with a fluorescein filter. The gel was then post-stained with SYBR Gold (Invitrogen) for 10 min to assess all RNAs in the reaction. The SYBR Gold filter on the ChemiDoc was used to visualize the hhRz, substrate, and cleavage products.

RNA folding. Two-dimensional RNA folding for substrate RNAs was predicted with RNAStructure (Version 6.3) (Reuter and Mathews, 2010) using a maximum energy difference of 10%, 20 as the maximum number of structures, and a window size of 2. RNA Composer (Popenda et al., 2012; Antczak et al., 2016) was used to predict three-dimensional RNA structure of substrates.

Data analysis. Statistical analysis and linear and nonlinear functional fitting were conducted in Origin software (OriginLab Corp., Northampton, MA). The criterion for significance was a p value of <0.05. In the analysis of upstream elements (FIG. 5A) and tetraloop elements (FIG. 5B) One-way ANOVA was used in the initial analysis as there is a single independent variable in each case with three or more independent groups, and with dependent variable being turnover rate. In the analysis of tetraloop composition (single independent variable) on turnover rate (dependent variable) One-way ANOVA was used in the initial analysis for both the 15-mer substrate (FIG. 6A) and the 14-mer substrate (FIG. 6B).

Quantitative microscopic Cellular mCherry2 Fluorescence imaging analysis. Open the Keyence BZ-X800 analysis software than open an epifluorescence image from the current experiment. Select hybrid cell count for batch analysis and under macro cell count click start. Open a macro stat conditions file that provides the threshold of fluorescence parameters. Add the epifluorescence images from the raw data file by pulling out images in the CH3 (TexasRed) channel then run the images against the conditions file and save the data. To process the saved data in Excel, open an Expedited Keyence VBA Template and save the experiment as a macro-enabled workbook excel file. Hold down Fn Alt F11 at the same time to open the VBA code for macro processing. Two windows will open, click the window that is titled Module2 (code) at the right you will see RunAll. Hold down Fn F5 to run the macrocode. It will open a window, select (but do not open) your results file folder. This will convert all CSV (Comma Separated Value) files into .xls files. Once the first portion of the code is complete, a new window will pop up. This time, open your results file folder, select the first file and hit ctr A to select all, then open the files. This will merge the results that are exported in separate workbooks into one workbook with separate sheets corresponding to each image. The average brightness integration data will automatically paste in the J9 cell in a single column on sheet 1. Average brightness integration data is collected and moved into Origin 2022 for statistical analysis. The Expedited Keyence VBA Template will be provided upon request to the authors.

### Example 2: Nuclease-Resistant Synthetic EhhRzs for Nucleic Acid Ocular Therapeutics

“Enhanced” refers to kinetics, and the EhhRzs developed and described herein are demonstrating orders of magnitude improvements over the expectations of hhRzs from the historical literature. The EhhRzs described herein can be made chemically stable with persistent kinetic activity, such that they can be utilized as injectable nucleic acid therapeutics, into the eye or other organs, or as gene-based therapeutics to be delivered by way of a vector. The EhhRzs described herein also have high enzyme activity and marked stability in nucleases. Synthetic biology can be paired with chemical biology to develop EhhRzs. Such new nucleotide incorporations are dependent upon chemical biological and synthetic biological principles, driven by molecular and atomic level knowledge about the interactions that drive high catalytic rate in EhhRzs. In addition, the EhhRz functionality has been moved into an RNA Pol-II expression construct which to allow cell-type specific promoter expression in the context of gene therapy. The results have demonstrated that the EhhRz is able to cleave itself out of an in cis construct and then attack other sites in the RNA in which it was embedded. After transfection of RNA Pol-II expression constructs, the data show that the EhhRz cleaves within the human intracellular milieu at cellular levels of Mg²⁺ using an efficient reporter assay.

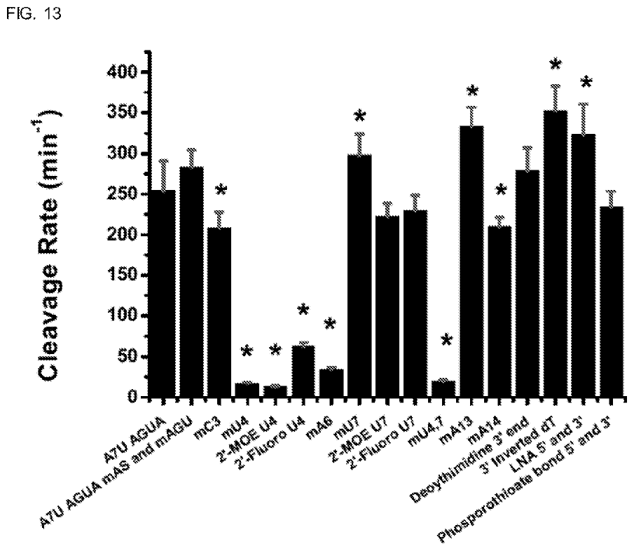

Chemically Stabilized Synthetic EhhRzs with Excellent Cleavage Kinetics. Synthetic EhhRzs (Syn-EhhRz) were investigated using commercially available first, second, and third generation nucleotide and backbone chemistries that are known to protect synthetic antisense (AS) agents from degradation. While AS has to bind stably to be effective as an mRNA suppression agent, the hhRz has to bind, undergo conformational changes to position important chemical residues, cleave the target, and then release products in order to initiate the next cycle of activity (turnover). The reaction cycle of the hhRz is much more complicated. Yet, the capacity for turnover of the ribozyme greatly exceeds the potential of an AS agent, which requires host cell machinery to cleave the target mRNA in the region of the target mRNA where the AS agent is bound. Earlier versions of chemically stabilized hhRz agents that were developed as blood stream injectable hhRzs for different diseases but did not succeed in clinical trials, and were slow, high Mg²⁺ requiring agents. Using model RHO 266, EhhRz A7U (AGUA) was identified as the most enhanced agent. It was determined whether the modifications would have impact on kinetic activity using the established real time kinetic assay against a 15-mer synthetic RNA substrate with 5′ FAM and 3′ BHQ1 which has suppressed fluorescence until cleaved followed by product release by the EhhRz. After tests for kinetic activity, nuclease resistance was tested. Modifications at various sites in the EhhRz RNA lead to a broad spectrum of kinetic activities with modifications at U4 and A6 within the hhRz enzyme core being generally deleterious to function (FIG. 13). It was surprising to find that some modifications enhanced turnover activity of the enzyme (e.g., 3′ Inverted dT). This behavior was similar to the findings of the range of kinetic activity with modifications of the composition of the Stem II loop (Example 1). A way to interpret these outcomes is that the specific atomic chemical environment immediately around the enzymatic core functionality (e.g., G12, G8, A9, A13) sets the stage for the reduction in activation energy that promotes enhanced catalytic activity. The results show that a range of chemistries and positions both near and more remote from the enzyme core can enhance rate. A potentially deleterious effect at one position may be offset by a beneficial effect at another. These findings show that the EhhRz can be made an even better enzyme through a combination of both chemical biological or synthetic biological approaches.

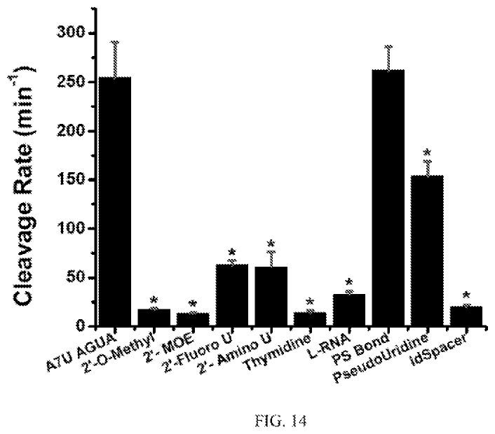

A broad range of synthetic chemistries were evaluated at the U4 position (FIG. 14). It was found that the first generation phosphorothioate bond in the linker 3′ to the U4 base does not influence activity with native U4, and that pseudouridine in the base component of U4 leads to strong enhanced kinetic activity (>50%) almost as good as the native uridine base, but is not protective against nucleases. In contrast, modifications of the 2′—OH group on the U4 ribose is generally inhibitory to activity: A DNA base (thymidine, 2′-H on the ribose) at U4 nearly inactivates the EhhRz, 2′O-Methyl and 2-MethoxyEthoxy T exert profound inhibition of activity, whereas modifications exerting a local structural impact such as a mirror image base (LNA) or a spacer strongly inhibit turnover activity. Some residual activity remains with 2′-Fluoro or 2′-Amino on the ribose sugar. These findings point to a marked effect of the 2′OH on the ribose of U4 in reaction chemistry and the structural environment around U4 in catalysis; the base uridine or pseudouridine has less effect and the charge distribution on the phosphodiester vs phosphorothioate link with G5 has no impact.

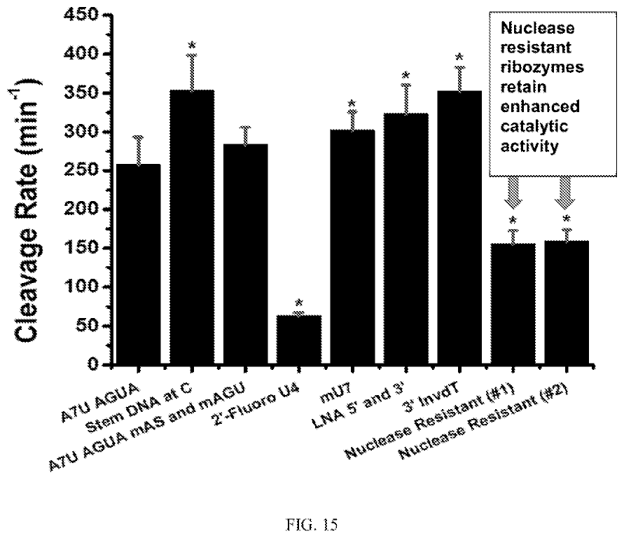

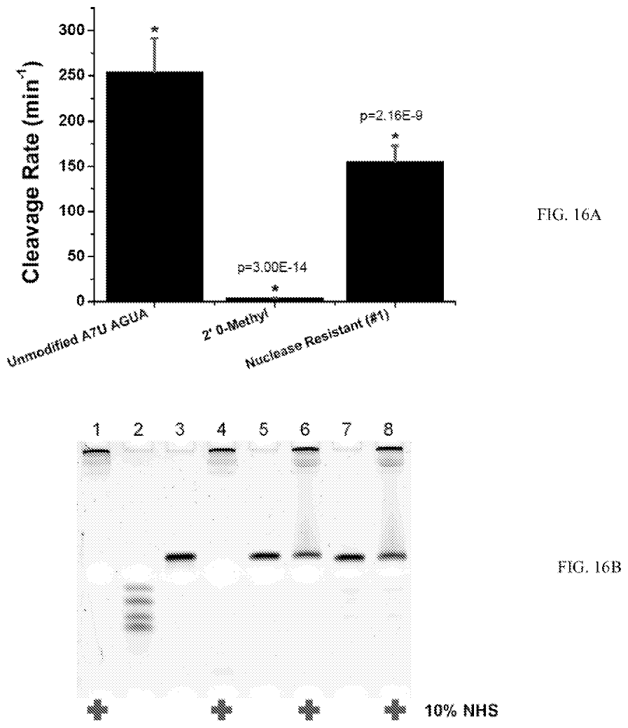

To develop an effective Syn-EhhRz, the turnover activity should be similar to or enhanced with respect to the native RNA chemistries and be resistant to nuclease degradation in both extracellular media (serum) and the intracellular environment, both of which contain a plethora of nucleases (RNA-endonucleases (e.g., RNase A) and RNA exonucleases (5′ to 3′; 3′ to 5′). Resistance to nuclease degradation increases the half-life of the Syn-EhhRz agent and leads to predictably greater number of kinetic turnover events. The native biological phosphodiester chemistry (G, A, U, C) of an EhhRz is completely degraded within 5 minutes in 10% human serum asserting a half-life less than 5 min. Nuclease sensitivity was assessed with polyacrylamide gel electrophoresis (PAGE). Within 5 minutes an EhhRz with a turnover rate of 250 nM/min would completely cleave 1 μM of small synthetic RNA target in vitro. If the half-life is extended to 24 hours the amount of target turnover would approach 300 μM, or at least two log-orders of magnitude greater impact at the level of target cleavage, a first order event in target knockdown, with the other variables being equal. Nucleic acid chemistries that allow resistance to both endonuclease and exonuclease degradation to enhance half-life without markedly influencing turnover activity identified (FIG. 15). The final compositions of a nuclease resistant Syn-EhhRz includes the following modifications. Nuclease Resistant (#1) LNA at both 5 and 3 prime ends, 2′ O-Methyl in the AS flanks and AGU of the cap, DNA at all Cs in stem-II 2′-Fluoro at U4 and 2′ O-Methyl at U7. Nuclease Resistant (#2) LNA at the 5 prime end, Inverted dT at the 3 prime end, 2′ O-Methyl in the AS flanks and AGU of the cap, DNA at all Cs in Stem-II, 2′-Fluoro at U4 and 2′ O-Methyl at U7 (blue arrows). To ascertain the relationship between catalytic activity and nuclease resistance, two Syn-EhhRz that have markedly different turnover rates were investigated (FIG. 16). While the 2-OMe (everywhere except G5, G8, G12, A9, A15.1, AL4) protects against nucleases it also kills enzyme activity. An aggregation of multi-generational protective chemistries provides for strongly enhanced activity (>150 min⁻¹) and strong nuclease resistance. The latter agent can be developed as an injectable Syn-EhhRz therapeutic for human RHO mRNA. The modifications described herein leading to nuclease resistance will carry over to other Syn-EhhRzs as they do not markedly impact the catalytic core chemistry.

A property identified which may prove to be very significant is that aggregating chemical modifications may lead to greater nuclease resistance, and as seen for the partially deleterious 2′-Fluoro U4 effect on turnover rate, this outcome was (partially) reversed in the aggregated Syn-EhhRz agent. This finding indicates that multiple chemical changes alter the structural composition of the enzyme core to influence catalysis.

Syn-EhhRzs sequences with chemical modifications that are tested in FIG. 15 are shown in Table 4.

TABLE 4
Sequences of Syn-EhhRzs.

| SEQ ID | | |
|---|---|---|
| NO: | Name | Sequence |
| 1 | Unmodified A7U AGUA | rGrGrGrUrUrGrArGrCrGrUrCrUrGrAr |
| | | UrGrArGrGrCrCrArGrUrArGrGrCrCrG |
| | | rArArArGrGrArArGrUrU |
| 89 | AZU GAAA 2′ O-Methyl | mGmGmGmUmUmGmAmGmCmGmUmCmUrGmAm |
| | throughout No G5, G8, G12, | UrGrAmGmGmCmCmGmAmArAmGmGmCmCrG |
| | A9, A15.1, L4 (Nuclease | mAmArAmGmGmAmAmGmUmU |
| | Resistant, No Catalytic | |
| | Activity) | |
| 90 | A7U AGUA stem DNA at C | rGrGrGrUrUrGrArGrCrGrUrCrUrGrAr |
| | | UrGrArGrGCCrArGrUrArGrGCCrGrArA |
| | | rArGrGrArArGrUrU |
| 91 | A7U AGUA 2′ O-Methyl AS | mGmGmGmUmUmGmAmGmCmGmUrCrUrGrAr |
| | flanks and AGU of stem loop | UrGrArGrGrCrCmAmGmUrArGrGrCrCrG |
| | II CAP | rArArAmGmGmAmAmGmUmU |
| 92 | A7U AGUA 2′-Fluoro U4 | mGmGmGmUmUmGmAmGmCmGmUrC/12FU/r |
| | (12FU) | GrArUrGrArGrGrCrCmAmGmUrArGrGrC |
| | | rCrGrArArAmGmGmAmAmGmUmU |
| 93 | A7U AGUA 2′ O-Methyl U7 | mGmGmGmUmUmGmAmGmCmGmUrCrUrGrAm |
| | | UrGrArGrGrCrCmAmGmUrArGrGrCrCrG |
| | | rArArAmGmGmAmAmGmUmU |
| 94 | A7U AGUA LNA (+, Locked | +G+G+GmUmUmGmAmGmCmGmUrCrUrGrAr |
| | Nucleic Acid) 5′ and 3′ | UrGrArGrGrCrCmAmGmUrArGrGrCrCrG |
| | | rArArAmGmGmAmA+G+T+T |
| 95 | A7U AGUA 3′ Inverted dT | rGrGrGrUrUrGrArGrCrGrUrCrUrGrAr |
| | (3InvdT) | UrGrArGrGrCrCrArGrUrArGrGrCrCrG |
| | | rArArArGrGrArArGrU/3InvdT/ |
| 96 | A7U AGUA LNA 5′ and 3′, | +G+G+GmUmUmGmAmGmCmGmUrC/i2FU/r |
| | 2′-Fluoro at U4, 2′ O-Methyl | GrAm UrGrArGrGCCmAmGmUrArGrGCCr |
| | U7 and AS Flanks and AGU of | GrArArAmGmGmAmA+G+T+T |
| | CAP, DNA at C in stem | |
| | (Nuclease Resistant #1) | |
| 97 | A7U AGUA LNA 5′ and 3′ | +G+G+GmUmUmGmAmGmCmGmUrC/i2FU/r |
| | Inverted dT, 2′-Fluoro at U4, | GrAmUrGrArGrGCCmAmGmUrArGrGCCrG |
| | 2′ O-Methyl U7 and AS Flanks | rArArAmGmGmAmAmGmU/3InvdT/ |
| | and AGU of CAP, DNA at C in | |
| | stem (Nuclease Resistant #2) | |

r = natural ribonucleotides (rG, rA, rU, rC);
m = 2′-OMe modified nts (mG, mA, mU, mC).
Stem II tetraloops are underlined.
3InvdT = inverted dT.
i2FU = 2′fluoro-uridine.
+Base = locked nucleic acid base.
Most of these sequence refer to EhhRz agents depicted in FIG. 15. 20Me refers to sequences in FIG. 16 (control inactive agent).

Chemical Modifications in Stem II and Loop. Phosphorothioate bonds between nucleotides within the Stem II are not protective against nuclease digestion in serum and phosphorothioate bonds in the Stem II loop have some impact on cleavage rate. Phosphorothioate bonds replacing phosphodiester bonds in the backbone were historically thought to create nuclease resistance. As described herein, under conditions used in the assays disclosed herein with 10% serum (or direct use of RNase A), that a phosphorothioate is not an absolute barrier to degradation, but perhaps it may slow the process under certain conditions.

Structure Function Properties Dependent upon the Length of Stem II and Composition of Stem II Loop. The interactions that occur between the loop capping Stem II within the core of the EhhRz and the 5′ end of the EhhRz base paired with the 3′ end of the substrate are important to achieving the highly active catalytic states that were measured and which depend importantly on the Stem II loop composition. As the distance between the upstream antisense flank (bound to substrate) with the Stem II loop (whatever its composition) involves atomic level distances and also partakes in conformational changes that support achieving the catalytically active state(s) of the enzyme, it was tested whether the length of Stem II would impact kinetic activity. Early evidence evaluating the impact of Stem II length on the kinetic activities of the model EhhRz suggest that the 4 bp length appears optimal with decreases in kinetic activity occurring with shortening or lengthening of Stem II. It has previously been shown that lengthening of Stem II to 6 bp was deleterious to activity of prior minimal hhRzs with 4 bp Stem II elements embedded in supportive RNA scaffolds (Yau et al., 2019) and extension to 8 bp and 13 bp obliterated activity with minimal hhRzs (Yau et al., 2019). The stem II loop appears to be an evolutionary variable as its size varies in natural occurring extended hhRzs in biology (Shepontovskaya and Uhlenbeck, 2008). Currently, the tetramer loops for stem II described herein show dramatic (log-orders) impact on kinetic activity (FIG. 6A, 6B). Historically, the minimal hhRz was thought to have a turnover activity maximized at 1-2 min⁻¹ (Emilsson et al., 2003; Breaker et al., 2003) and that the maximized enzyme activity was a constant. The data described herein shows that the kinetic activity is highly dependent on the Stem loop II composition (e.g., AGUA maximum). This finding is consistent with an evolutionary perspective in that the various extended hhRzs that evolved to perform specific but varied functions in different organisms, with varied turnover and religation rates, likely reflect the finding that the enzyme kinetic function is not a constant, but rather can be evolutionarily tuned to the needs of the organism. Surprisingly, the variation in turnover rate with composition of Stem II loop is similar to what we have seen with diverse protective chemistries. An exploration of the structural accessibility of the various experimentally testing Stem II loops shows similar accessibility and no relationship to the experimental turnover activity of the enzymes; in other words it is the chemistry of the loop composition and the interactions that those chemistries generate, which is the reason for the log-orders variation in kinetics.

Synthetic Nucleic Acids as Therapeutics. In Example 1, the EhhRz chemistry was native RNA (use of G, A, U, C nucleotides). RNA catalysis can be highly efficient even with constraints. One constraint is that the pKa's of important residues such as G8 and G12 in the enzyme core are well away from neutrality and limit efficient proton transfer reactions important to catalysis at physiological pH. For comparison, RNase A is a highly efficient enzyme that uses the same mechanism as the hhRz but with amino acid chemistry; two His residues (pKa's near physiological neutrality) drive the mechanism of general base and general acid chemistry catalysis. While the mhhRz is expected to be limited to 1-2/min, the results show turnover rates of EhhRzs as high as 250/min or 4/sec (>2 log enhancement). This clearly demonstrates that RNA catalysis can enter into the realm of protein based enzymatics (>10/sec). Also, EhhRzs which present conditions that allow a hhRz core enzyme to perform with efficiency (kcat/Km) at least as good as RNase A, but if the kcat (now maximized at around 600/min) can be enhanced then efficiency could be much higher with an equivalent Km. RNaseA has a kcat on the scale of 50,000/min. to the results described here provide evidence that synthetic nucleic acid catalysts can be used as therapeutics. For example, the chemistry that optimizes function (e.g., picking replacements for important core enzyme residues (e.g., G8, G12) that have pKa's closer to physiological pH) that will more efficiently drive the proton exchange reactions that underlie reaction chemistry can be selected.

The results described herein also provide evidence that EhhRzs can be used as injectable synthetic nucleic acid therapeutics. The compositions disclosed herein can be formulated for intravitreal injections of small chemically protected antisense agents (and can diffuse and distribute in the vitreous gel, cross the internal limiting membrane (a primary barrier to viral vectors such as AAV), cross the retina proper, reach photoreceptors, enter these cells, and finally enter the nucleus to manifest splice switching function on mutant pre-mRNAs; also, such therapeutics can have long term protective effects (stability) and clinical impact in patients. No scaffold would be needed for this approach, which keeps the sequence length to around 40 nts. Syn-EhhRzs can be used to target any intraocular disease target. For example, EhhRzs can be designed to target PRPH2 mRNA, transcribed by a gene which when mutated causes a variety of autosomal dominant macular and retinal degenerations.

In sum, the data described herein support the development of EhhRzs as therapeutics, for example, opsin-mediated disease processes such as Dry AMD or autosomal dominant retinitis pigmentosa. The therapeutic EhhRz can be used as an injectable therapeutic or as a packaged gene-based therapeutic. While some antisense agents that are injected into the vitreous of the eye in the context of retinal degenerations recently (e.g., Murray et al., 2015; Cideciyan et al., 2019) have been used, EhhRzs, as described herein go beyond an antisense agent because they are have both antisense and catalytic function that is a capacity for target turnover at lower levels of therapeutic agent. These kinetically enhanced agents with up to two log orders improved turnover functionality demonstrate that the EhhRz does function in human cells. Also, therapeutic expression constructs that embrace the additional factor of co-localization with target mRNA can be used to enhance collision encounter frequency which is important to the second order annealing reaction that precedes cleavage.

The results disclosed show that the upstream region of the EhhRz and the Stem II tetraloop can be modulated to optimize enhanced catalytic turnover function against accessible target regions of arbitrary target mRNAs or viral RNAs.

The results disclosed herein also show that EhhRzs have proven capacity to function to cleave structured target mRNA regions in the intracellular environment of free cellular Mg²⁺, ionic strength, viscosity, and temperature.

Further, synthetic chemistries can be used both to protect the EhhRz from degradation due to RNases in extracellular (e.g., serum) or intracellular media, and chemistries can be identified that further enhance catalytic function.

Synthetic protections of EhhRzs can allow carry over nuclease resistance to arbitrary EhhRzs because they do not engage the nucleotides that promote the chemical process of target mRNA cleavage.

Example 3: Effects of EhhRzs in vitro and in cells. A cDNA expression construct was produced for the readily assayed reporter, mCherry2, which contained four structured 266 EhhRz targeting sites in hRHO which are 51 nt in length and fold as stem loop structures. The 2D predicted single stranded region of these stem loop structures contains the 266 CUCd cleavage site. One 266 stem loop structure was inserted in the 5′ UTR of mCherry2 and three were inserted into the 3′ UTR of mCherry2. Then, mCherry2 cDNA was appended. Expressed from a CMV promoter in cells, the EhhRzs have capacity to cleave at target sites in cis to the fusion reporter-target mRNA. Cleavage at any of the sites in the UTRs is expected to reduce the half-life of the mRNA and thereby reduce the overall mCherry reporter protein which gets translated.

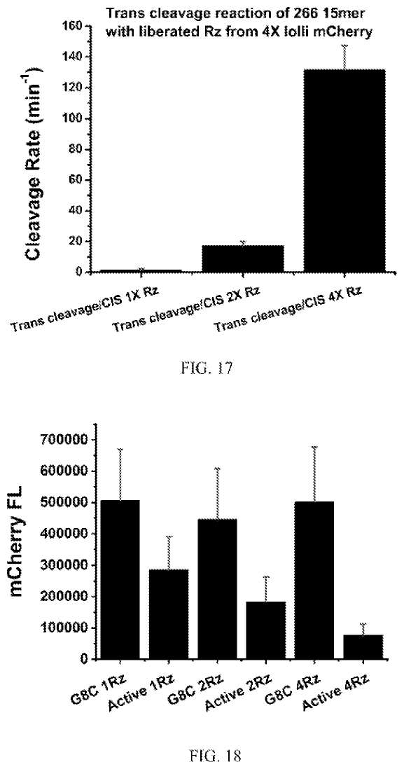

The data show that an in vitro transcribed fusion reporter-target mRNA generates cleavage products which are capable of cleaving a 15-nt substrate 266 hRHO mRNA target (1 μM), in trans, in the moderate throughput fluorescence turnover assay (FIG. 17). The turnover rate measured is clearly proportional to the number of EhhRzs embedded in the 3′UTR of the fusion-Reporter mRNA. With 4 EhhRzs present, the overall turnover rate begins to approach the level seen in fluorescence assays where an EhhRz is at 100 nM and substrate is at 1 μM. The molarity of the self-cleaved RNA put into the assay was 100 nM. These data demonstrate that the functional EhhRz has reached the range between 10-100 nM.

Next, the results show that cells transfected with CMV expression constructs for the mCherry-266 stem loop structure reporter construct with 1, 2, or 4 active or inactive EhhRzs demonstrated progressive decreasing levels of the mCherry fluorescence (protein) with increasing numbers of active EhhRzs in the 3′UTR of the mRNA (FIG. 18). The level of mCherry expression with constructs expressing catalytically inactive EhhRzs was unchanged. This result shows that the EhhRz can function within the intracellular environment of the human cell at physiological levels of Mg²⁺, ionic strength, viscosity, and temperature. It also shows that the catalytic core mutation used (G8C) has a marked impact on the capacity of the EhhRz to function and that the progressive suppression of mCherry fluorescence was due to a catalytic effect and not an antisense effect (an antisense effect is still possible in the catalytically inactive EhhRzs, but there was no progressive suppression with increasing number of inactive EhhRzs in the construct). These results provide evidence that the disclosed hammerhead ribozymes can knockdown intracellular proteins levels.

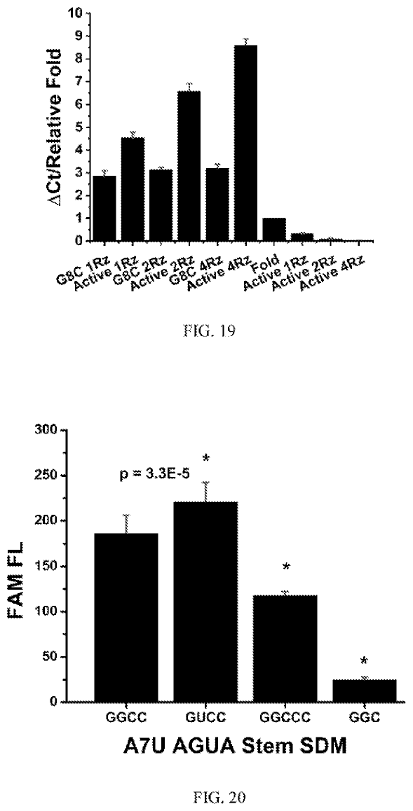

The impact of EhhRz using the fusion-target expression construct on the mCherry mRNA levels in transfected HEK293S cells was investigated (FIG. 19). After total RNA purification and cleanup to remove DNA, Fusion mRNA was quantified using RT-PCR referenced to β-Actin as a housekeeping mRNA and analyzed by the 2(−ΔΔCt) method using a primer set established for mCherry. ΔCt relationship between mCherry and β-Actin increases with the number of active EhhRzs appended (ΔCt: 1 EhhRz=1.7, 2 EhhRzs=3.4 and 4 EhhRzs=5.4). The relative fold analysis by the 2(−ΔΔCt) method reveals the % of detectable mCherry with appended EhhRzs compared to the inactive G8C control (1 EhhRz=31% (69% knockdown), 2 EhhRzs=10% (90% knockdown) and 4 EhhRzs=2% (98% knockdown).

In sum, the data demonstrates that EhhRzs have a strong capacity to cleave themselves out of mRNAs by targeting a human hRHO RNA element and promoting exceptionally strong knockdown at the mRNA level of the fusion-target-reporter mRNA and at the protein levels. Active EhhRzs are important for these events which downplays the possible role of pure antisense effects. EhhRzs are proven to function under intracellular conditions of the human cell where Mg²⁺ levels are at physiological concentrations, and where ionic strength, viscosity, and temperature are physiological in value. Additional data demonstrates that the EhhRz is cleaving target both within the nucleus of the cell and the cytoplasm, a finding that has important relevance to design of purely in trans cleaving therapeutic constructs.

Effects of stem II variants. The effects of structural variations on stem II which supports the tetraloop element was also investigated (FIG. 20). A fluorescence assay was used with the model unstructured 15-nt substrate to assess turnover rate under conditions of physiological Mg²⁺ and temperature. The hammerhead ribozyme comprising stem 5′-GGCC-AGUA-GGCC-3′ (SEQ ID NO: 17), shown with an AGUA tetraloop created 4 bp of stability and was GC rich. Creating a more flexible stem with GUCC-AGUA-GGAC (SEQ ID NO: 18) significantly enhances the overall turnover cleavage rate in vitro with short model substrate; this stem, however, is naturally occurring. Extending the original stem GGCCC-AGUA-GGGCC (SEQ ID NO: 19) to a 5 bp GC rich decreases activity, while shortening the stem to 3 bp GGC-AGUA-GCC (SEQ ID NO: 20) markedly inhibits activity. These findings show that the stability, length, and likely the chemistry of stem II can contribute in a major way to turnover activity of EhhRzs.

Methods. A structured RNA target (51 nt) derived from human rhodopsin mRNA (hRHO) contains a hhRz cleavage site (CUC↓) at position 266. Using a fusion RNA reporter, four copies (1 in 5′UTR, 3 in 3′UTR) of this target RNA element was embedded into an mCherry mRNA expression construct. Subsequently, 1, 2, or 4 copies of an EhhRz targeting the 266 cleavage site downstream of the 3′UTR target elements was also embedded. A target (1-532 nt) of hRHO was also generated for in vitro transcription that contains the native 266 CUC↓. In vitro T7pol co-synthesis/cleavage reactions were analyzed by PAGE urea gels. In vivo functional assays measured suppression of the mCherry fluorescence by quantitative imaging (Keyence). mCherry mRNA was measured by RT-PCR with the 2(−ΔΔCt) comparative method to β-actin and represented as the relative fold of the G8C control. Cell fractionation buffer enriched fractions with RNA isolated by TRIzol and RNeasy Plus Mini Kit. Catalytically inactivated EhhRzs and hardened mRNA targets prevent cleavage. Statistical analysis performed in Origin. Graphics generated in BioRender.com.

In conclusion, the results show EhhRzs can bind to and cleave targets in cis or in trans after being liberated by self-cleavage out of the fusion mRNA transcript. EhhRzs function within the intracellular environment to cleave NUH↑ sites within large structured targets where physiological conditions of free Mg2+, temperature, ionic strength, and viscosity exist. EhhRzs also strongly suppress mCherry protein expression by cleavage in untranslated regions that affect mRNA half-life. EhhRzs in cis can modulate target knockdown by the number of EhhRzs appended. EhhRzs have the capacity for catalytic function in the nucleus and/or cytoplasm.

### Example 4: Comparison of a 266 hRHO EhhRzs to Gorbatyuk et al. (2007) 525 mhhRz

In an allele-independent therapeutic strategy for autosomal dominant disease (e.g., autosomal dominant retinitis pigmentosa or adRP) a ribozyme (or other post-transcriptional gene silencing agent, e.g., shRNA) is identified which targets an accessible site present in most mutant mRNAs for the given gene; but, this therapeutic will also target the wild type (WT=normal) mRNA. Therefore, while a single therapeutic could treat a single gene, the loss of the WT mRNA and protein could stress the cell and promote its own toxicity in addition to the mutant protein. Therefore, in the allele-independent therapeutic strategy the therapeutic ribozyme or shRNA expression (or delivery) element must be paired with a WT expression element that produces an mRNA that cannot be cut by the therapeutic agent, so it reconstitutes the WT protein to avoid that potential element of toxicity (known as haploinsufficiency). This knockdown: reconstitute or ablate: replace strategies for autosomal dominant diseases is the focus of current research.

Gorbatyuk et al. (2007; Gorbatyuk M, et al. Exp. Eye Res. 2007; 84: 44-52) used a minimal hhRz (mhhRz) targeting site 525 of mutant mouse P23H RHO mRNA in a rat transgenic P23H RHO adRP model and achieved a partial rescue of retinal (photoreceptor) degeneration associated with reduction in the mutant P23H RHO mRNA after subretinal adeno-associated virus (AAV) delivery of the therapeutic transgene as part of an allele-independent therapeutic strategy. In the Gorbatyuk et al. (2007) study, a mutant mouse P23H RHO mRNA and protein expressed from a transgene in rat rod photoreceptors was used and this mutant P23H RHO protein promotes death of rat rod photoreceptors. The 525 mhhRz reduced (approximately 46%) the mutant mouse P23H RHO mRNA but not the rat WT RHO mRNA (different targeting sequence) which decreased the rate of loss of electroretinogram (ERG) parameters for photoreceptors and reduced the rate of loss of photoreceptor nuclei in the retina. So, the mhhRz 525 was partially protective in the rat adRP model. The disease continued to progress but was slowed by the therapeutic delivery of mhhRz 525. The 525 targeting sequence in the mouse P23H mRNA is homologous to a sequence present in both canine and human RHO mRNAs; rat RHO mRNA has a sequence difference over the targeting region which is protective against the 525 mhhRz.

The 525 mhhRz was also tested in a natural occurring dog mutant T4R RHO adRP model of photoreceptor degeneration (Cideciyan et al., 2018; Cideciyan A V, et al. Proc. Natl. Acad. Sci. USA 2018; 115(36): E8547-E8556). The results showed that very high doses of AAV delivering the 525 mhhRz expression construct were needed to generate log-order reduction of normal dog RHO mRNA and protein. When tested in the mutant T4R RHO dog adRP model, the very high dose delivery promoted some patches of photoreceptor protein whereas a lower dose which did not reduce the mRNA/protein as extensively was not protective. However, in the high dose testing in the dog adRP model there was evidence of toxicity seen as severe signs of retinitis/chorioretinitis. The 525 mhhRz was dropped from development in the large scale mammalian model testing, which then focused on a shRNA knockdown/reconstitute strategy which showed greater potency and less toxicity relative to the 525 AAV-mhhRz strategy.

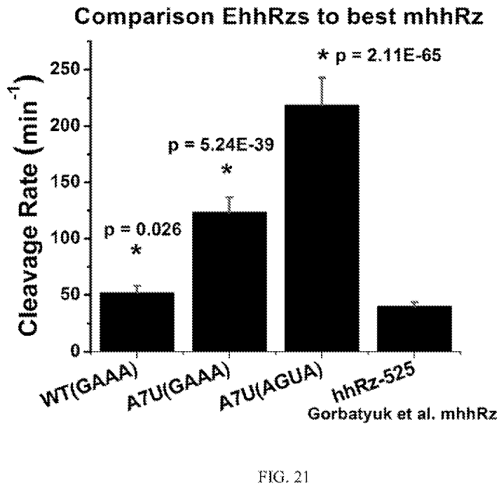

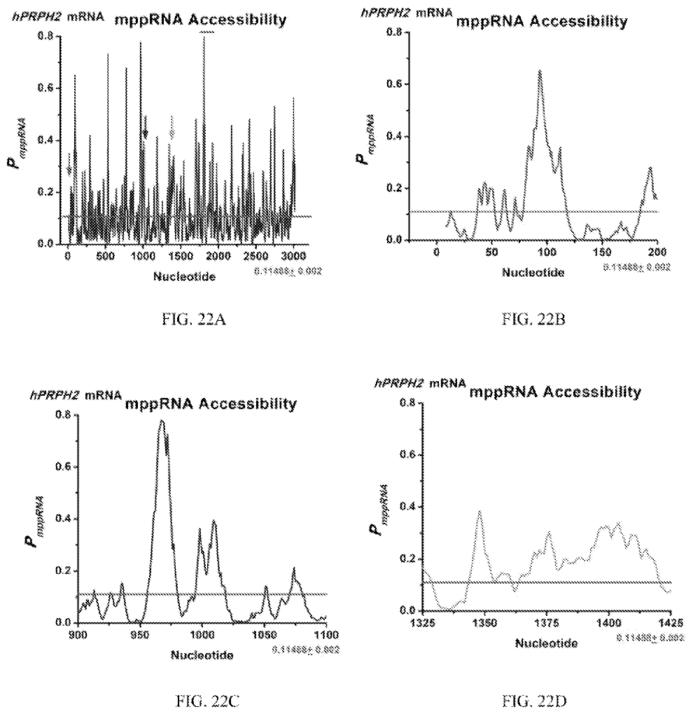

Given the partial successes in both the rat and dog adRP models with 525 mhhRz, three EhhRzs (e.g., WT(GAAA), A7U(GAAA), and A7U(AGUA) were compared against hRHO mRNA to the 525 mhhRz agent in a head-to-head experimental study. It was tested whether the limited protective effectiveness of the 525 mhhRz agent in the rod and canine models of RHO adRP could be due to poor kinetic efficiency of the 525 mhhRz. The turnover activity of mhhRz 525 was compared to the 266 hRHO EhhRzs (e.g., WT(GAAA), A7U(GAAA), and A7U(AGUA), using their individual short model substrates, in a moderate throughput real time fluorescence assay (FIG. 21). Rates for the comparisons were scaled to independent analysis of product fluorescence per mole. The three EhhRzs targeting CUC 266 in hRHO were statistically more effective than the 525 mhhRz against its substrate. The EhhRz 266 [A7U(AGUA)] is 1.5-log more efficient (32-fold) than mhhRz 525 (1.89×10⁸ M⁻¹min⁻¹ vs 5.13×10⁶ M⁻¹ min⁻¹) in the assay than the 525 mhhRz. The 525 mhhRz had a Kₘ of 152 nM and k_{cat} of 0.78 min⁻¹ under [S]>[E] conditions at 2 mM Mg²⁺ and 37° C. (Gorbatyuk et al., 2007). The Kₘ of our enzyme is 1,344 nM, but the rate of enzyme turnover (k_{obs}) at even lower levels of Mg²⁺ (0.5 mM) is 254 min⁻¹. When compared at 2 mM Mg²⁺, the EhhRz 266 [A7U(AGUA)] has a turnover rate of approximately 428 min⁻¹.

The markedly increased turnover efficiency reflects the enzymatic capacity of EhhRzs, and substrate accessibility between the two model targets. An agent with such improved turnover capacity can attack and cleave more target mRNA molecules that a hhRz agent with much lower turnover capacity. This means that greater knockdown of target mRNA and protein can occur with lower levels of expression and/or delivery. Lower levels of expression/delivery will make for lower risks of toxicity. The overall expected impact will have a greater efficacy/toxicity ratio as a therapeutic. Given these findings, the EhhRz 266 [A7U(AGUA)] can be used a therapeutic in RHO dependent adRP.

### Example 5: PRPH2 and TCF4 Enhanced Hammerhead Ribozymes

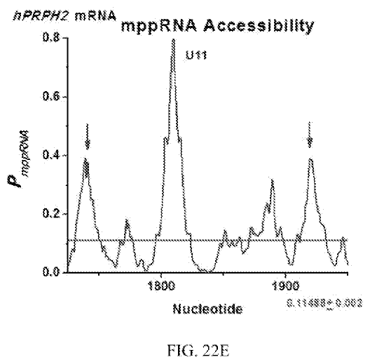

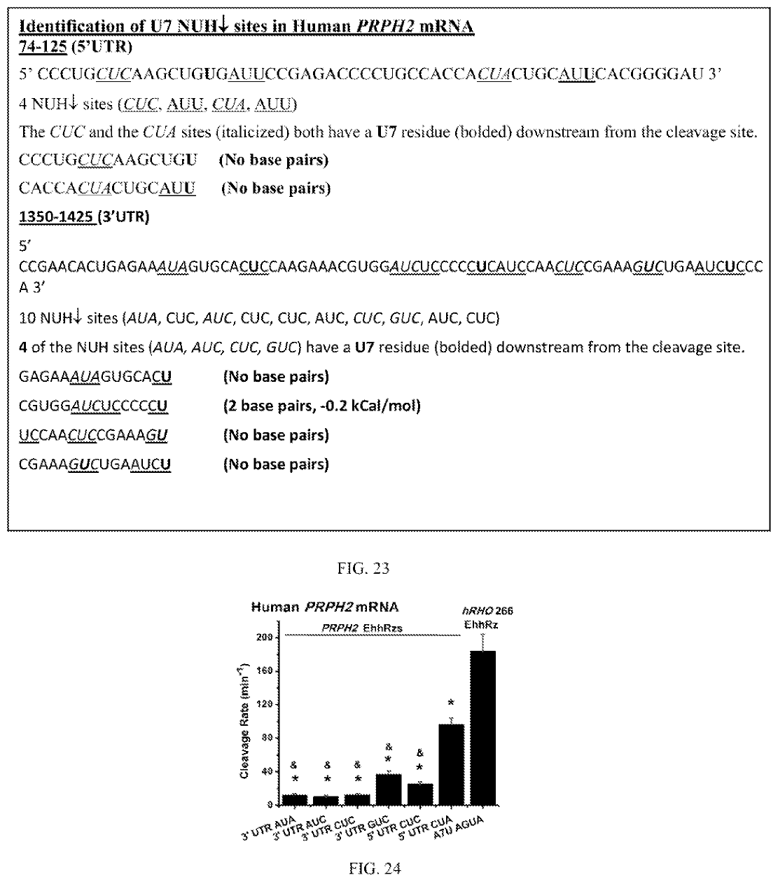

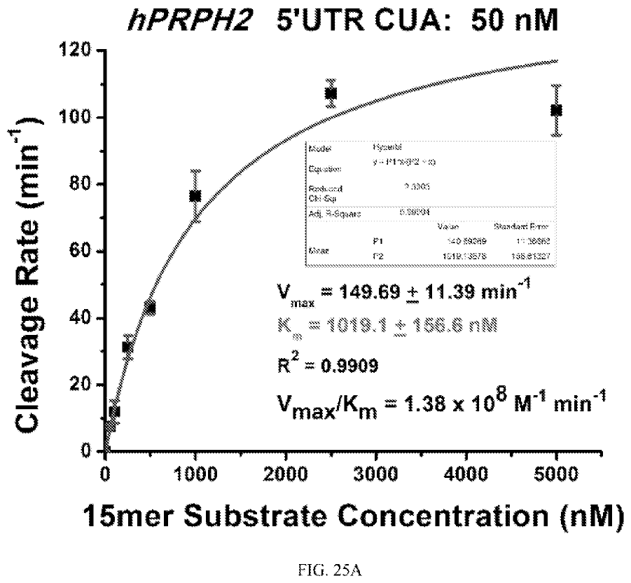

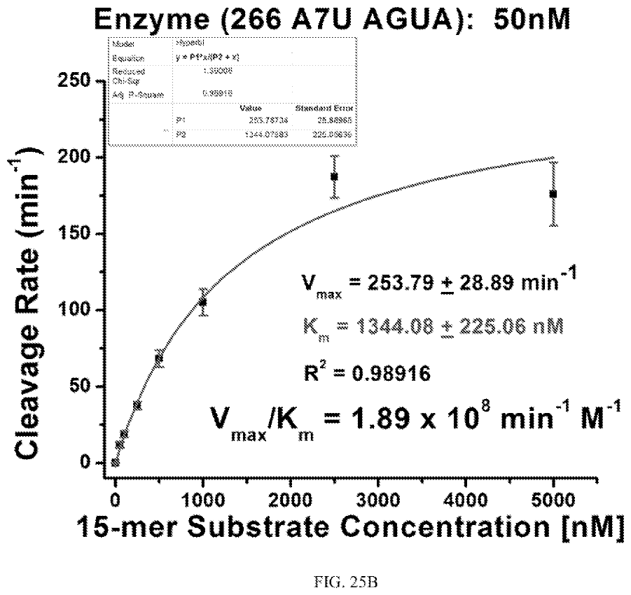

Human PRPH2 mRNA EhhRz Development. A bioinformatics mppRNA analysis of human PRPH2 mRNA was conducted to identify accessible regions in the mRNA fold (FIG. 22). Regions of accessibility were identified which were peaks of accessibility that exceed the average accessibility across the mRNA. The 5′UTR and 3′UTR accessible regions were of particular focus. The NUH↓ sites were identified within those regions that contained a U7 residue downstream of the NUH↓ site and have no or little secondary structure in the short model substrates (FIG. 23). Using a real time fluorescence assay, an experimental screen was conducted for hPRPH2 EhhRzs against 15-mer substrates having various NUH↓ sites and a U7 residue downstream of the cleavage site (FIG. 24). The PRPH2 hhRzs used the AGUA tetraloop identified for high hRHO EhhRz activity and the A7U unpaired upstream nucleotide; they had different numbers of 5′G residues to avoid enzyme structural trapped states (which are also inhibitory). Each had significantly different kinetics from the product-scaled 266 CUC A7U(AGUA) EhhRz against hRHO. Each of the PRPH2 hhRzs had significantly different mean rates when compared to the 5′UTR CUA↓ EhhRz against PRPH2, which was identified as a lead agent against this human retinal degeneration target. This 115 CUA↓ A7U(AGUA) hPRPH2 agent was clearly above the 10/min threshold at approximately 100/min. The CUC↓ site in the 5′UTR and the GUC↓ site in the 3′UTR of hPRPH2 were also above initial threshold and can be used for combinatorial therapeutics. A Michaelis-Menten analysis was conducted to characterize the lead hPRPH2 EhhRz (FIG. 25) and found a Vmax turnover rate and Km on the same scale as the hRHO 266 A7U(AGUA) EhhRz. The enzyme efficiency is also similar (1.38×10⁸ M⁻¹ min⁻¹ vs. 1.89×10⁸ M⁻¹ min⁻¹, respectively). These enzyme efficiencies are both on the scale of the protein enzyme RNaseA (1.4×10⁸ M⁻¹ min⁻¹) which uses the same reaction mechanism to cleave the phosphodiester bond as a hhRz. There are four mechanisms by which a phosphodiester cleaving enzyme is thought to promote enhanced rate. RNaseA is thought to use the four mechanisms, the minimal hhRz is thought to use two mechanism, and that the EhhRz is able to capture additional processes (perhaps the 4 mechanism) for rate acceleration to make it perform similar to RNaseA. It is noted that RNaseA has much faster kinetics than the EhhRz on the order of 10,000 min⁻¹, so while the efficiency of EhhRzs and RNaseA is in the same class, the speed of cleavage is still much faster with amino acid sidechains than with nucleotide sidechains. To improve the EhhRz, for example, it is contemplated to alter the pKa's of G12 and G8 with synthetic nucleotides; these residues are important to two of the four aspects of speed acceleration.

Mutations in human PRPH2 cause a variety of retinal and macular degenerations, generally of an autosomal dominant type of inheritance: dominant retinitis pigmentosa (RP), dominant macular dystrophy, dominant adult vitelliform macular dystrophy, dominant cone-rod dystrophy, dominant central areolar choroidal dystrophy, and autosomal recessive Leber Congenital Amaurosis. Mutations in PRPH2 account for about 5% of autosomal dominant RP. The PRPH2 protein traffics to the outer segment of both rod and cone photoreceptors and is important in forming the disc structures that support the visual pigments of rods and cone and the phototransduction microenvironment. Like autosomal recessive null mutations in the carrier state (heterozygous) for hRHO autosomal recessive mutations in the carrier state for PRPH2 are also well tolerated and do not promote retinal degeneration. This shows that 50% of wild type hPRPH2, like 50% of WT hRHO are sufficient to keep photoreceptor cells alive and functional. This has important implications for therapeutics.

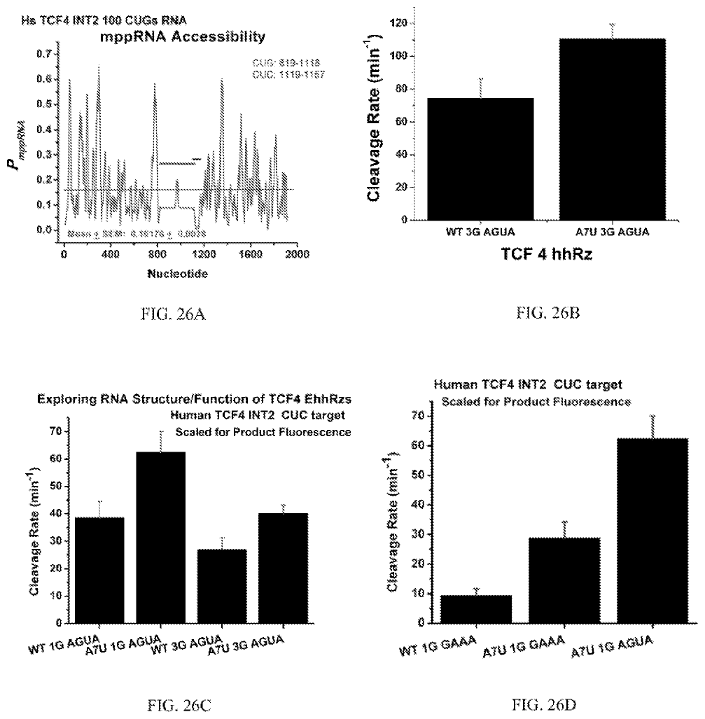

Human TCF4 mRNA EhhRz Development. Fuchs endothelial corneal dystrophy (FECD) is known to be caused by a CUG expansion in the second intron of the TCF4 gene. The early application of EhhRzs to FECD was investigated by attack of a CUCA motif in the second intron downstream of the poly CUG repeat (FIG. 26). Mapping of accessibility showed the impact of the poly CUG repeat on mRNA structure. Comparison of the WT (comprising an A7U substitution) AGUA and A7U AGUA EhhRzs showed the benefit of the A7U variation, as found for hRHO EhhRzs. With this EhhRz the presence of 1G at the 5′ end was more beneficial than 3Gs. The evolution from WT(GAAA) to A7U(GAAA) to A7U(AGUA) in terms of increasing turnover rate followed the path seen for our best 266 hRHO EhhRzs.

In sum, the development of EhhRz has been extended from hRHO to PRPH2 to TCF4. This diversifies the disease mRNA targets that are accessible using this technology described herein.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A hammerhead ribozyme comprising a stem II region, and a stem terminator loop, wherein the stem terminator loop comprises the nucleic sequence of AGUA, UAAA, AAAA, GUGA, GAGA, GAUA, GUUA, GACA, AAAG, AACA, AUUA, AAUA, or AGCA.

2. The hammerhead ribozyme of claim 1, further comprising a first flanking region at the 5′ end, wherein the first flanking region comprises an A7U substitution.

3. A hammerhead ribozyme comprising a stem II region, and a stem terminator loop, and a first flanking region at the 5′ end, wherein the first flanking region comprises an A7U substitution.

4. The hammerhead ribozyme of claim 3, further comprising a second flanking region at the 3′ end.

5. The hammerhead ribozyme of any of the preceding claims, wherein the hammerhead ribozyme comprises an enzyme core.

6. The hammerhead ribozyme of any of the preceding claims, wherein the hammerhead ribozyme has an increased turnover rate of a substrate compared to a wild-type hammerhead ribozyme.

7. The hammerhead ribozyme of any of claims 2-6, wherein the first flanking region and the second flanking region comprise a first and a second complementary nucleotide sequence to a target mRNA sequence.

8. The hammerhead ribozyme of claim 6, wherein the target mRNA sequence comprises a hammerhead ribozyme cleavage site, wherein the hammerhead ribozyme cleavage site has the nucleotide sequence of NUH, wherein N is any nucleotide, U is U, and H is C, A or U.

9. The hammerhead ribozyme of claim 6, wherein the target mRNA sequence encodes rhodopsin, peripherin-2, centrosomal, or transcription factor 4.

10. The hammerhead ribozyme of any of the preceding claims, wherein the hammerhead ribozyme is nuclease resistant.

11. An hammerhead ribozyme comprising a nucleic acid sequence of CUGAUGA GGCC X₁X₂X₃X₄ GGCC GAA (SEQ ID NO: 29), wherein X₁X₂X₃X₄ of SEQ ID NO: 29 are AGUA, UAAA, AAAA, GUGA, GAGA, GUUA, GAUA, GACA, AAAG, AACA, AUUA, AAUA, or AGCA, and wherein the nucleic acid sequence has an enhanced turnover rate compared to a wild-type hammerhead ribozyme.

12. The hammerhead ribozyme of claim 11, further comprising a first flanking region located on the 5′ end of the hammerhead ribozyme and a second flanking region located on the 3′ end of the hammerhead ribozyme, wherein the first and second flanking regions comprise a first and a second complementary nucleotide sequence to a target mRNA sequence.

13. The hammerhead ribozyme of claim 12, wherein the first flanking region comprises from 5′ to 3′ the nucleic acid sequence of GGGUUGAGCGU (SEQ ID NO: 45), and the second antisense flanking region comprises from 5′ to 3′ the nucleic acid sequence AGGAAGUU.

14. The hammerhead ribozyme of claim 12, wherein the first and the second complementary nucleotide sequences of the first flanking region and the second flanking regions bind to a target mRNA sequence.

15. The hammerhead ribozyme of claim 12, wherein the hammerhead ribozyme comprises the nucleic acid sequence of GGGUUGAGCGUCUGAUGAGGCCX₁X₂X₃X₄GGCCGAAAGGAAGUU (SEQ ID NO: 3), wherein X₁X₂X₃X₄ of SEQ ID NO: 3 are AGUA, UAAA, AAAA, GUGA, GAGA, GUUA, GAUA, GACA, AAAG, AACA, AUUA, AAUA, or AGCA, and wherein the nucleic acid sequence has an enhanced turnover rate compared to a wild-type hammerhead ribozyme.

16. The hammerhead ribozyme of claim 11, wherein the hammerhead ribozyme is nuclease resistant.

17. The hammerhead ribozyme of any of claims 12 to 16, further comprising one or more modified nucleobases.

18. The hammerhead ribozyme of claim 17, wherein the one or more modified nucleobases is a 2′-O-methyl, a 2′-amino, a 2′-Fluoro, a 2′-O-methoxy-ethyl, a phosphorothioate bond, a 2′-Fluoro, a locked nucleic acid, or an inverted dT.

19. A plasmid or vector comprising any of the ribozymes of claims 1-18.

20. A pharmaceutical composition comprising the hammerhead ribozymes of any of claims 1-19.

21. The pharmaceutical composition of claim 20, wherein the hammerhead ribozymes are formulated in a delivery vehicle.

22. The pharmaceutical composition of claim 20, wherein the hammerhead ribozymes are formulated for intraocular, intravitreal, subretinal, suprachoroidal, intracameral, or subconjunctival administration.

23. A method of treating retinitis pigmentosa in a subject, the method comprising administering to the subject a therapeutically effective amount of any of the hammerhead ribozymes of any of claims 1-20 or the pharmaceutical composition of claims 21-23.

24. A method of treating retinal dystrophy in a subject, the method comprising administering to the subject a therapeutically effective amount of any of the hammerhead ribozymes of any of claims 1-20 or the pharmaceutical composition of claims 21-23.

25. A method of treating dry macular degeneration in a subject, the method comprising administering to the subject a therapeutically effective amount of any of the hammerhead ribozymes of any of claims 1-20 or the pharmaceutical composition of claims 21-23.

26. A method of increasing the turnover rate of a substrate, the method comprising administering to a subject a therapeutically effective amount of any of the hammerhead ribozymes of any of claims 1-20 or the pharmaceutical composition of claims 21-23.

27. The method of claim 26, wherein the target mRNA sequence comprises a hammerhead ribozyme cleavage site, wherein the hammerhead ribozyme cleavage site has the nucleotide sequence of NUH, wherein N is any nucleotide, U is U, and H is C, A or U.

28. The method of claim 27, wherein the target mRNA sequence encodes rhodopsin, peripherin-2, centrosomal, or transcription factor 4.

29. The method of claim 27, wherein the subject is identified as being in need of treatment before the administration step.

30. The method of claim 27, wherein the subject has retinitis pigmentosa, retinal dystrophy, primary open angle glaucoma, corneal dystrophies, or dry macular degeneration.

31. The method of any of claims 23-30, wherein the hammerhead ribozyme or the pharmaceutical composition is administered intraocularly, intravitreally, subretinally, suprachoroidal, intracameral, or subconjunctivally.
